SEQUENCE LISTING (167 sequences)

SEQ ID NO: 1 - protein, 392 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKE

SEQ ID NO: 2 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 4 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 5 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 6 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 7 - protein, 1101 aa, Actinomyces naeslundii
MWYASLMSAHHLRVGIDVGTHSVGLATLRVDDHGTPIELLSALSHIHDSGVGKEGKKDHDTRKKLSGIARRARRLLHHRRTQLQQLDEVLRDLGFPIPTPGEFLDLNEQTDPYRVWRVRARLVEEKLPEELRGPAISMAVRHIARHRGWRNPYSKVESLLSPAEESPFMKALRERILATTGEVLDDGITPGQAMAQVALTHNISMRGPEGILGKLHQSDNANEIRKICARQGVSPDVCKQLLRAVFKADSPRGSAVSRVAPDPLPGQGSFRRAPKCDPEFQRFRIISIVANLRISETKGENRPLTADERRHVVTFLTEDSQADLTWVDVAEKLGVHRRDLRGTAVHTDDGERSAARPPIDATDRIMRQTKISSLKTWWEEADSEQRGAMIRYLYEDPTDSECAEIIAELPEEDQAKLDSLHLPAGRAAYSRESLTALSDHMLATTDDLHEARKRLFGVDDSWAPPAEAINAPVGNPSVDRTLKIVGRYLSAVESMWGTPEVIHVEHVRDGFTSERMADERDKANRRRYNDNQEAMKKIQRDYGKEGYISRGDIVRLDALELQGCACLYCGTTIGYHTCQLDHIVPQAGPGSNNRRGNLVAVCERCNRSKSNTPFAVWAQKCGIPHVGVKEAIGRVRGWRKQTPNTSSEDLTRLKKEVIARLRRTQEDPEIDERSMESVAWMANELHHRIAAAYPETTVMVYRGSITAAARKAAGIDSRINLIGEKGRKDRIDRRHHAVDASVVALMEASVAKTLAERSSLRGEQRLTGKEQTWKQYTGSTVGAREHFEMWRGHMLHLTELFNERLAEDKVYVTQNIRLRLSDGNAHTVNPSKLVSHRLGDGLTVQQIDRACTPALWCALTREKDFDEKNGLPAREDRAIRVHGHEIKSSDYIQVFSKRKKTDSDRDETPFGAIAVRGGFVEIGPSIHHARIYRVEGKKPVYAMLRVFTHDLLSQRHGDLFSAVIPPQSISMRCAEPKLRKAITTGNATYLGWVVVGDELEINVDSFTKYAIGRFLEDFPNTTRWRICGYDTNSKLTLKPIVLAAEGLENPSSAVNEIVELKGWRVAINVLTKVHPTVVRRDALGRPRYSSRSNLPTSWTIE

SEQ ID NO: 8 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 9 - protein, 1334 aa, Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 10 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 11 - protein, 1084 aa, Corynebacterium diphtheriae
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 12 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 13 - protein, 1073 aa, Rhodobacteraceae bacterium
MRLGLDIGTNSIGWWLCETDRADARVRINGVLAGGVRIFSDGRDPKSRASLAVDRRAARAMRRRRDRYLRRRATLMKVLANAGLMPSTPEEAKALELLDPYELRATGLDQILPLTHLGRALFHINQRRGFKSNRKTDWGDNESGKIKDATARLDLAILANGARTYGEFLHKRRQRAVDPRHVPTVRTRLSIANRDGPDGKEEAGYDFYPDRKHLEEEFRKLWAAQANFHPELTEDLHDLIFEKIFYQRPLKEPKVGLCLFTSEERLPKAHPLTQARVLYETVNQLRVIADGRETRRLTLEERDQIIYVLDNKKPTVSLKSMAMKLPALARTLKLRDGERFTLETGVRDAIACDPVRSSLSHPDRFGPRWSTLDATAQWEVVSRVRKVQSEAEHAALVDWLMQAYSIDRNHAEATANAPLPEGFGRLGQTATTSILERLKADVVTYAEAVAACGWHHSDQRTGECLDRLPYYGEVLDRHVIPGTYDANDDEVTRYGRITNPTVHIGLNQLRRLVNRIIETYGKPDQIVLELARELKQSEQQKRDAIKRIRDTTEAAKKRSEKLEELGIEDNGRNRMLLRLWEDLNPEDAMRRFCPYTGERISATMIFDGSCDVDHILPYSRTLDDSFANRTLCLKEANREKRNQTPWKAWGDAPKWDTIEAKLKNLPENKRWRFAPDAMERFEGEKDFLDRALVDTQYLARISRTYMDTLFSEGGHVWVVPGRLTEMLRRHWGLNSLLSDKDRGAVKAKNRTDHRHHAIDAAVVAATDRSLLNRISRAAGQGEAAGQSAELIARDTPPPWEGFRDDLRVQLDKIIVSHRADHGRIDREGRKQGRDSTAGQLHNDTAYGVVDAMTVVSRTPLLSLKPSDIAVTPKGKNIRDPQLQKALEIATRGKEGKAFEAALRQFAEKAGAYQGLRRVRLIETLQESARVEIGTRSEGGPLKAYKGDSNHCYELWRLPDGKVKPQVVTTYEAHAGIEKRPHPAAKRLLRTFKRDMVALERNGETVICYVQKFNQAGILFLASHLESNADARDRDPNDSFTLFRMSPGPMHKAGIRRVSVDEIGRLRDGGAETH

SEQ ID NO: 14 - protein, 965 aa, Campylobacter coli
MKIIGFNLGIANIGWALRENDEIIDCGVRVFDIPENPKNGNSLALERRENKARMKIVKRKKARMLATKTFLKKEFNVDLSKLFLIGSTQSIYELRTKALSSLISKEELSAIILHIAKHRGYDDSALKNENGTIIEALNKNKEAMLKFKSVGEYFYKNFVQNKEVKKIRNTTEDYSNSVPRSLLKQELDLILDKQKELGLIKNADFKAKLFEIIFFKRPLKDFSNKIGNCIFFENEKRAAKNTISACEFVALGKVVNLLKSIEKDIGIVYEKDSINEIMSIILDKTSISYKKIRDILNLPQDINFKGLDYSKNNVENSKLVDLKKLNEFKKALGDGFTNLDKDILDSIATDITLTKDTATLKEKLKNYNVLNAEQIEKLSELVFNDHINLSLKALKQIIPLMYEGKRYDEACELCNFTIAKNQEKNEYLPLFEKTRFAKDISSPVVIRAICEFRKLLNDIIRRYGSVHKIHLELTRDFGISFNDRKKIIKEIEQNEQSRIKALETIKELKLEETSKNIQIVRLFEDQKGICPYSGLKMDLKCLDELVIDYIRPYNRSLDDSYSNKVLTFKKLNDLKQGKTPFEAFGEDEKLWAEINERIKEYNGKKRFKIFDKFFKDKKPFDFTEQTLQDTRWLTKLVASYLNEYLSFLPISEDENTALGYGEKGSKQHVILSSGMITQMLRNFWYLGFKNHKDYKNNAMDAIIVAFTTNSIIFTFNNFKKELDLAKAEFYANKISESDYLLKRKFLPPFSGFKEQALEKVKNIFVSHSLKIKNKGTLHELTPLKIKELKNTYGDLDLAVKLGKIRKYNDKYYANAKGSLVRTDLFVDKENKFHAVSIYKADFSTKKLPNKTPATTSNGETKEGIEMNENYNFCMSLYKNTPIGVKIKGMKESIICYYHGFNTSGSKITYKKHDNNYHNLSEDEMVVFRKNDKESIVVGKILEIKKYSISPSGELSLIENEKRKWF

SEQ ID NO: 15 - protein, 1466 aa, Ignavibacteria bacterium
MKNILGLDLGTNSIGWALIDKENNKIIDMGSRIIPMSQDILGEFGKGNSISQTAERTNYRSIRRLRERYLLRRERLHRVLNILEFLPKHYSDQIDFETRLGKFKEDTEPKIAYKSTIDETNSKSRFDFIFKKSFAEMLEDFHQYQPELFANDNKIPYDWTIYFLRKKALTKKIEKEELAWILLNFNQKRGYYQLREELEEDTNKKEYVVSLKVIKIVKGEEDKKNKNRNWYSISLENGWVYNATFSTEPQWLMTEKEFLVTEELDENGQVKIVKDKKSDKEGKEKRRIIPLPSFDEINLMSKSEPDRIYKKIKAKTETAISNSGKTVGEYIYENLLQNPSQKIRGKLIRTIERKFYKEELKQILQKQKEFHPELQNDDLYNDCVRELYKNNEGHQFLLSKRDFIHLLLDDIIFYQRPLKSQKSLISNCTFEFKKYNVGNEEKIKYLKAIPKSHPLYQEFRFWQWIYNLRVYRKDDDQDVTNDYLNDPEKYADLFEFLSNRKEIDQKALLKYFKLKESTHRWNFVEDKKYPCFETRTLISTRLEKVKDLPPNFLTDQTELQLWHIIYSVTDKIEFEKALSTFAKRNKLDVTTFVENFKKFPPFKSEYGSYSGKALKKLLPLMRSGRYWKWDDIDEKTKTRIDKIITGEFDEDIKNKVREKSINLTTENHFQGLQVWLASYIVYDRHAEAATINKWDTIEHLENYIKEFKQHSLRNPIVEQVTLEALRVIKDIWKQFGKSAENFFDEIHIELGREMKNTADERKRLTSQINDNENTNVRIKALLAELKNDSNIENVRPFSPIQQELLKIYEDGVLNSEIEIPDDISKISKTAQPSSSELQRYKLWLEQKYRSPYTGQVIPLAKLFTTDYEIEHIIPQSRYFDDSFNNKVICEAAVNKLKDNQTGLEFIKNHHGEIVQTVFDNKVKIFEENDYRDFVKTHYIKNRSKRNKLLMEEIPDKMIERQINDTRYITKFISALLSNIVRAENNDEGLNSKNLIQVNGKITSLLRQDWGINDIWNDLILPRFLRMNQITNSDAFTRYNDKYQKYLPTVPLELSKNYQSKRIDHRHHALDALIIACATRDHVNLLNNKYAKSKERYDLNRKLRLFEKVVYTHPKTGEKIEREIPKNFIKPWDTFTVDTKNFLDTIVVSFKQNLRIINKATNQYQKWVKLNGRNVKKEVKQSGINWAIRKPLHKETVAGKVELKRIKVPKGKILTATRKNLDTSFDIKTIESITDTGIQKILKNYLSAKGNDPTIAFSPEGIEEMNKNITRYNNGKPHRPIYKARIFELGSKFILGLTGNKKAKYVEAAKGTNLFYAIYVDENNKRSFETIPLNIVIERQKQGLSSVPENDDKGNKLLFYLSPNDLVYVPDEDEIINESYLDVSNLSNEQKKRLYNVNDFSSTCYFTPNRIAKAIAPKEVDLNYDNNKKKLFGSYDTKTASVNGIQIKDICIKLKADRLGNISKANR

SEQ ID NO: 16 - protein, 1319 aa, Fructobacillus sp.
MGYNIGLDIGTGSVGWAALTDEGKLARAKGKNLIGVRLFDSAQSAAQRRSYRTTRRRLSRRKWRLRLLENIFSDEMGMIDENFFARLKYSYVHPKDEVNNAHYYGGYLFPTQQETHDFHEKFQTIYHLRLKLMIEDCKFDLREIYLAMHHIVKYRGHFLNSQSKMTIGDSYNPRDFQQAIQNYAEAKGLIWSLNDAQEMTDVLVGQAGFGLSKKAKAERLLSAFSFDTKEDKKAIQAILAGIVGNTTDFTKIFNRERSGDELKKWKLKLDSEAFDEQSQAIVDELDDDEMELFNAIRQAFDGFTLMDLLGDQTSISAAMVKRYQQHHDDLKMVKEIAKKQGLSHQDFSKIYTAFLKDDTDKGMKALLDKADLADDVLVEIQQRIESHDFLPKQRTKANSVIPYQLHLAELEKIIENQGKYYPFLLDTFTNKAGETINKLVELVKFRVPYYVGPMVTAADVEKAGGDATNHWVKRNEGYEKSPVTPWNFDQVFNRDQAAQDFIDRLTGTDTYLIGEPTLLKNSLKYQLFTVLNELNNVKINGHKIDEKTKHVLIQDLFKSKKTVSEKAIKDYYLSQGMGEIQIVGLADKTKFNSNLSSYIDLSKTFDAEFMENPANQELLENIIQIQTVFEDVKIAERELQKLALPDEQVQQLAKTHYTGWGNLSDKLLSTPIIQEGSQKVSILNKLQTTSKNFMSIITDNKFGVQQWIQEQNTAETADSIQDRIDELTTAPANKRGIKQAFNVLFDIQKAMGEEPNRVYLEFAKETQNSVRTNSRYNRLKDLYKSKTLSDDVKALKEELESQKSSLQSERIGDRLYLYFLQQGKDMYTGQPINIDKLSTDYDIDHIIPQAYTKDDSIDNRVLVSRPENARKSDSATYTTEVQQSAGGLWKSLKNAGFISQKKYDRLTKGGDYSKGQKTGFIARQLVETRQIIKNVASLIESEFSQTKAVAIRSEITADMRRLVAIKKHREINSFHHAFDALLITAAGQYMQARYPDRDGANVYNEFDYYTNTYLKELRQSSSSSQVRRLKPFGFVVGTMAKGNENWSEDDTQYLRHVMNFKNILTTRRNDKDNGALNKETIYAVDPKAKLIGTNKKRQDVSLYGGYIYPYSAYMTLVRANGKNLLVKVTISAAEKIKSGQIELSEYVQQRPEVKKFEKILINKLAIGQLVNNDGNLIYLTSYEFYHNAKQLWLPTEEADLISQLNKDSSDEDLIKGFDILTSPAILKRFPFYELDLKKLVNIRDKFIAVENKFDILMVILKALQLDAAQQKPVKMIDKKSADWKDYRQRGGIKLSDTSEIIYQSTTGIFEKRVKISNLL

SEQ ID NO: 17 - protein, 1274 aa, Pedobacter glucosidilyticus
MTKHILGLDLGTNSIGWAIIQVDNNNNVPIQIIAMGSRIIPLDSNDRDQFQKGQAISKNKDRTTARTQRKGYDRKQLKKSDDFKYSLKKILEKLDIFPTEELMKLPTLDLWKLRSDAVSNIEDITPKQLGRILYMLNQKRGYKSARSEANADKKDTDYVAEVKGRYTQLKDKGQTLGQYFYKELSDANQNNTYYRVKEKVYPREAYIEEFDAIINVQKSKHSFLTDEVIHSLRNEIIYYQRKLKSQKGLVSICEFEGFETTYFDKKTQQDKTIFTGPKVAPRTSPLFQFCKIWEVVNNISLKTKNPEGSKYKWSDRIPTIEEKQTIANYLQENENLSFIELLKILQLKKEQVYANKQILKGIQGNTTFSAIHKIIGNSEHLKFDIETIPSKHFAVLVDKKTGEILDERDSLELNSALEQEPFYQLWHTIYSIKDLDECKKALIKRFNFEEEIAEKLSKIDFNKQAFGNKSNKAMRKMLPYLMLGYNQSEAESFAGYNRRLTKEEKSKNVSDEPLQLLAKNSLRQPVVEKILNQMINVVNAIIEKYGKPEEIRVELARELKQSKDEREDADKQNGFNKKLNELVATKLTELGLPTTKHYIQKYKFIFPAKDKNWKEAQVANQCIYCGDTFNLTEALSGDNFDVDHIVPKALLFDDSQANKVLVHRSCNSTKTNNTAYDYITKKGSQALNDYVARVDDWFKRGIISYGKMQRLKVSFEEYQERKKIGKETEADKRIWENFIDRQLRETAYIAKKAKEILEKVCHNVTSTEGNVTAKLRQLWGWDNVLMNLQLPKYKELEKKTKQTFTQLKEWTSDHGNRKHQKEEIINWTKRDDHRHHAIDALVIACTQQGFIQRINTLSSSDVKDEMKKELEEDKTVYNERLTLLENYLLEKKPFSTEEIEKEADKILVSFKAGKKVATLSKYKATGINEIKGVLVPRGPLHEQSVYGKIKVIEKDKPLKYLFENSDKIVNPLIKHLVKTRLLENENNAQAALVTLKNKPILLNNKQTEILEKASCYNEATVLKYKLQSLKASQIDDIVDEKIKFLIKERLSKFGNKEKEAFKDILWFNEKKQIPITSIRLFARPDANNLQVIKKHEKGKNIGFVLSGNNHHIAIYEDKNNKLIQHICDFWHAVERKRNNIPVLIEDTSTIWNHLINEDFSESFLNKLPNDSLKLKFSLQQNEMFILGLPKEQSEEAIKSNNKSLLSKHLYLVWSITDGDYFFRHHLETKNTELKKIDGSKESKRYLRLSTKSLVDLNPIKVRLNHLGEITKIGE

SEQ ID NO: 18 - protein, 1082 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 19 - protein, 7 aa, Simian virus 40
PKKKRKV

SEQ ID NO: 20 - protein, 16 aa, unidentified
  REGION 1..16: note = Description of Unknown: nucleoplasmin NLS sequence
KRPAATKKAGQAKKKK

SEQ ID NO: 21 - DNA, 20 bp, synthetic construct
  misc_feature 1..20: note = Description of Artificial Sequence: Synthetic oligonucleotide
  source 1..20: mol_type = other DNA
gtaacggcagacttctcctc

SEQ ID NO: 22 - DNA, 20 bp, synthetic construct
  misc_feature 1..20: note = Description of Artificial Sequence: Synthetic oligonucleotide
  source 1..20: mol_type = other DNA
gtctgccgttactgccctgt

SEQ ID NO: 23 - DNA, 20 bp, synthetic construct
  misc_feature 1..20: note = Description of Artificial Sequence: Synthetic oligonucleotide
  source 1..20: mol_type = other DNA
gaggtgaacgtggatgaagt

SEQ ID NO: 24 - DNA, 20 bp, synthetic construct
  misc_feature 1..20: note = Description of Artificial Sequence: Synthetic oligonucleotide
  source 1..20: mol_type = other DNA
tatctgtctgaaacggtccc

SEQ ID NO: 25 - DNA, 20 bp, synthetic construct
  misc_feature 1..20: note = Description of Artificial Sequence: Synthetic oligonucleotide
  source 1..20: mol_type = other DNA
gctaaactccacccatgggt

SEQ ID NO: 26 - DNA, 20 bp, synthetic construct
  misc_feature 1..20: note = Description of Artificial Sequence: Synthetic oligonucleotide
  source 1..20: mol_type = other DNA
caaggctattggtcaaggca

SEQ ID NO: 27 - DNA, 20 bp, synthetic construct
  misc_feature 1..20: note = Description of Artificial Sequence: Synthetic oligonucleotide
  source 1..20: mol_type = other DNA
aaataagaatgtcccccaat

SEQ ID NO: 28 - DNA, 20 bp, synthetic construct
  misc_feature 1..20: note = Description of Artificial Sequence: Synthetic oligonucleotide
  source 1..20: mol_type = other DNA
cacaaacggaaacaatgcaa

SEQ ID NO: 29 - DNA, 20 bp, synthetic construct
  misc_feature 1..20: note = Description of Artificial Sequence: Synthetic oligonucleotide
  source 1..20: mol_type = other DNA
aatatcatttctgttcaaaa

SEQ ID NO: 30 - DNA, 20 bp, synthetic construct
  misc_feature 1..20: note = Description of Artificial Sequence: Synthetic oligonucleotide
  source 1..20: mol_type = other DNA
taataattgatgtcatagat

SEQ ID NO: 31 - DNA, 20 bp, synthetic construct
  misc_feature 1..20: note = Description of Artificial Sequence: Synthetic oligonucleotide
  source 1..20: mol_type = other DNA
tgacatcaattattatacat

SEQ ID NO: 32 - DNA, 20 bp, synthetic construct
  misc_feature 1..20: note = Description of Artificial Sequence: Synthetic oligonucleotide
  source 1..20: mol_type = other DNA
ctttttatttatgcacaggg

SEQ ID NO: 33 - DNA, 20 bp, synthetic construct
  misc_feature 1..20: note = Description of Artificial Sequence: Synthetic oligonucleotide
  source 1..20: mol_type = other DNA
atcccctccatggtaaccgc

SEQ ID NO: 34 - DNA, 20 bp, synthetic construct
  misc_feature 1..20: note = Description of Artificial Sequence: Synthetic oligonucleotide
  source 1..20: mol_type = other DNA
acttacactgatcccctcca

SEQ ID NO: 35 - DNA, 20 bp, synthetic construct
  misc_feature 1..20: note = Description of Artificial Sequence: Synthetic oligonucleotide
  source 1..20: mol_type = other DNA
ggagaggatggcccggcggc

SEQ ID NO: 36 - DNA, 20 bp, synthetic construct
  misc_feature 1..20: note = Description of Artificial Sequence: Synthetic oligonucleotide
  source 1..20: mol_type = other DNA
atggcccggcggctggcccg

SEQ ID NO: 37 - DNA, 20 bp, synthetic construct
  misc_feature 1..20: note = Description of Artificial Sequence: Synthetic oligonucleotide
  source 1..20: mol_type = other DNA
ggatggcccggcggctggcc

SEQ ID NO: 38 - DNA, 20 bp, synthetic construct
  misc_feature 1..20: note = Description of Artificial Sequence: Synthetic oligonucleotide
  source 1..20: mol_type = other DNA
taggtatgcaaaataaatca

SEQ ID NO: 39 - DNA, 20 bp, synthetic construct
  misc_feature 1..20: note = Description of Artificial Sequence: Synthetic oligonucleotide
  source 1..20: mol_type = other DNA
catacctaatcattatgctg

SEQ ID NO: 40 - DNA, 20 bp, synthetic construct
  misc_feature 1..20: note = Description of Artificial Sequence: Synthetic oligonucleotide
  source 1..20: mol_type = other DNA
taaattctttgctgacctgc

SEQ ID NO: 41 - DNA, 20 bp, synthetic construct
  misc_feature 1..20: note = Description of Artificial Sequence: Synthetic oligonucleotide
  source 1..20: mol_type = other DNA
tgtagccctctgtgtgctca

SEQ ID NO: 42 - DNA, 20 bp, synthetic construct
  misc_feature 1..20: note = Description of Artificial Sequence: Synthetic oligonucleotide
  source 1..20: mol_type = other DNA
aactagaatgaccagtcaac

SEQ ID NO: 43 - DNA, 20 bp, synthetic construct
  misc_feature 1..20: note = Description of Artificial Sequence: Synthetic oligonucleotide
  source 1..20: mol_type = other DNA
gatgatctctcaactttaac

SEQ ID NO: 44 - DNA, 20 bp, synthetic construct
  misc_feature 1..20: note = Description of Artificial Sequence: Synthetic oligonucleotide
  source 1..20: mol_type = other DNA
cactaaagcagaatcgcaaa

SEQ ID NO: 45 - DNA, 20 bp, synthetic construct
  misc_feature 1..20: note = Description of Artificial Sequence: Synthetic oligonucleotide
  source 1..20: mol_type = other DNA
tgcctttaccttgcgtccac

SEQ ID NO: 46 - DNA, 20 bp, synthetic construct
  misc_feature 1..20: note = Description of Artificial Sequence: Synthetic oligonucleotide
  source 1..20: mol_type = other DNA
cctgtcagtcttcatgctgt

SEQ ID NO: 47 - DNA, 20 bp, synthetic construct
  misc_feature 1..20: note = Description of Artificial Sequence: Synthetic oligonucleotide
  source 1..20: mol_type = other DNA
tctgctaggtcctaccatcc

SEQ ID NO: 48 - DNA, 20 bp, synthetic construct
  misc_feature 1..20: note = Description of Artificial Sequence: Synthetic oligonucleotide
  source 1..20: mol_type = other DNA
ctttcacaatctgctagcaa

SEQ ID NO: 49 - DNA, 20 bp, synthetic construct
  misc_feature 1..20: note = Description of Artificial Sequence: Synthetic oligonucleotide
  source 1..20: mol_type = other DNA
aaattctgaatcggccaaag

SEQ ID NO: 50 - DNA, 20 bp, synthetic construct
  misc_feature 1..20: note = Description of Artificial Sequence: Synthetic oligonucleotide
  source 1..20: mol_type = other DNA
cggccaaagaggtataattc

SEQ ID NO: 51 - DNA, 20 bp, synthetic construct
  misc_feature 1..20: note = Description of Artificial Sequence: Synthetic oligonucleotide
  source 1..20: mol_type = other DNA
attctttatagactgaattt

SEQ ID NO: 52 - DNA, 20 bp, synthetic construct
  misc_feature 1..20: note = Description of Artificial Sequence: Synthetic oligonucleotide
  source 1..20: mol_type = other DNA
gctcagtactgctgtagaat

SEQ ID NO: 53 - DNA, 20 bp, synthetic construct
  misc_feature 1..20: note = Description of Artificial Sequence: Synthetic oligonucleotide
  source 1..20: mol_type = other DNA
tgctcagtactgctgtagaa

SEQ ID NO: 54 - DNA, 20 bp, synthetic construct
  misc_feature 1..20: note = Description of Artificial Sequence: Synthetic oligonucleotide
  source 1..20: mol_type = other DNA
gaaggacttgagggactcga

SEQ ID NO: 55 - DNA, 20 bp, synthetic construct
  misc_feature 1..20: note = Description of Artificial Sequence: Synthetic oligonucleotide
  source 1..20: mol_type = other DNA
agcggctgtgcctgcggcgg

SEQ ID NO: 56 - DNA, 20 bp, synthetic construct
  misc_feature 1..20: note = Description of Artificial Sequence: Synthetic oligonucleotide
  source 1..20: mol_type = other DNA
gcgtaccacacccgtcgcat

SEQ ID NO: 57 - DNA, 20 bp, synthetic construct
  misc_feature 1..20: note = Description of Artificial Sequence: Synthetic oligonucleotide
  source 1..20: mol_type = other DNA
cgagtacccacagtactacc

SEQ ID NO: 58 - DNA, 20 bp, synthetic construct
  misc_feature 1..20: note = Description of Artificial Sequence: Synthetic oligonucleotide
  source 1..20: mol_type = other DNA
cctgtggtccttggtggtcc

SEQ ID NO: 59 - DNA, 20 bp, synthetic construct
  misc_feature 1..20: note = Description of Artificial Sequence: Synthetic oligonucleotide
  source 1..20: mol_type = other DNA
atattttctttaatggtgcc

SEQ ID NO: 60 - DNA, 20 bp, synthetic construct
  misc_feature 1..20: note = Description of Artificial Sequence: Synthetic oligonucleotide
  source 1..20: mol_type = other DNA
tctgtatctatattcatcat

SEQ ID NO: 61 - DNA, 20 bp, synthetic construct
  misc_feature 1..20: note = Description of Artificial Sequence: Synthetic oligonucleotide
  source 1..20: mol_type = other DNA
gtggtacctctggtggcggg

SEQ ID NO: 62 - DNA, 20 bp, synthetic construct
  misc_feature 1..20: note = Description of Artificial Sequence: Synthetic oligonucleotide
  source 1..20: mol_type = other DNA
gctagctgtggcagtggccc

SEQ ID NO: 63 - DNA, 20 bp, synthetic construct
  misc_feature 1..20: note = Description of Artificial Sequence: Synthetic oligonucleotide
  source 1..20: mol_type = other DNA
gaaggtggcgttgtcccctt

SEQ ID NO: 64 - DNA, 20 bp, synthetic construct
  misc_feature 1..20: note = Description of Artificial Sequence: Synthetic oligonucleotide
  source 1..20: mol_type = other DNA
atgtggaagtcacgcccgtt

SEQ ID NO: 65 - DNA, 20 bp, synthetic construct
  misc_feature 1..20: note = Description of Artificial Sequence: Synthetic oligonucleotide
  source 1..20: mol_type = other DNA
ccttggatttcagcggcaca

SEQ ID NO: 66 - DNA, 20 bp, synthetic construct
  misc_feature 1..20: note = Description of Artificial Sequence: Synthetic oligonucleotide
  source 1..20: mol_type = other DNA
tgcatactcacacacaaagc

SEQ ID NO: 67 - DNA, 20 bp, synthetic construct
  misc_feature 1..20: note = Description of Artificial Sequence: Synthetic oligonucleotide
  source 1..20: mol_type = other DNA
agctgtttctttgagcaaaa

SEQ ID NO: 68 - DNA, 20 bp, synthetic construct
  misc_feature 1..20: note = Description of Artificial Sequence: Synthetic oligonucleotide
  source 1..20: mol_type = other DNA
cggctccatcctctggctcg

SEQ ID NO: 69 - DNA, 20 bp, synthetic construct
  misc_feature 1..20: note = Description of Artificial Sequence: Synthetic oligonucleotide
  source 1..20: mol_type = other DNA
ccttcacattccgtgtctcc

SEQ ID NO: 70 - DNA, 20 bp, synthetic construct
  misc_feature 1..20: note = Description of Artificial Sequence: Synthetic oligonucleotide
  source 1..20: mol_type = other DNA
cctgcgctcttggaccgcgg

SEQ ID NO: 71 - DNA, 20 bp, synthetic construct
  misc_feature 1..20: note = Description of Artificial Sequence: Synthetic oligonucleotide
  source 1..20: mol_type = other DNA
ctgagccgccatgtccgccg

SEQ ID NO: 72 - DNA, 20 bp, synthetic construct
  misc_feature 1..20: note = Description of Artificial Sequence: Synthetic oligonucleotide
  source 1..20: mol_type = other DNA
gcaggaggggccggagtatt

SEQ ID NO: 73 - DNA, 20 bp, synthetic construct
  misc_feature 1..20: note = Description of Artificial Sequence: Synthetic oligonucleotide
  source 1..20: mol_type = other DNA
tggacgacacccagttcgtg

SEQ ID NO: 74 - DNA, 20 bp, synthetic construct
  misc_feature 1..20: note = Description of Artificial Sequence: Synthetic oligonucleotide
  source 1..20: mol_type = other DNA
ctctccgctgctccgcctca

SEQ ID NO: 75 - DNA, 20 bp, synthetic construct
  misc_feature 1..20: note = Description of Artificial Sequence: Synthetic oligonucleotide
  source 1..20: mol_type = other DNA
gatctgagccgccgtgtccg

SEQ ID NO: 76 - DNA, 20 bp, synthetic construct
  misc_feature 1..20: note = Description of Artificial Sequence: Synthetic oligonucleotide
  source 1..20: mol_type = other DNA
gtagaacaaaaaaaaagacc

SEQ ID NO: 77 - DNA, 20 bp, synthetic construct
  misc_feature 1..20: note = Description of Artificial Sequence: Synthetic oligonucleotide
  source 1..20: mol_type = other DNA
tgggcactgttgctgvctgg

SEQ ID NO: 78 - DNA, 20 bp, synthetic construct
  misc_feature 1..20: note = Description of Artificial Sequence: Synthetic oligonucleotide
  source 1..20: mol_type = other DNA
gagagactcatcagagccct

SEQ ID NO: 79 - DNA, 20 bp, synthetic construct
  misc_feature 1..20: note = Description of Artificial Sequence: Synthetic oligonucleotide
  source 1..20: mol_type = other DNA
cttcctcctacacatcatag

SEQ ID NO: 80 - DNA, 20 bp, synthetic construct
  misc_feature 1..20: note = Description of Artificial Sequence: Synthetic oligonucleotide
  source 1..20: mol_type = other DNA
tagcggtgaccacagctcca

SEQ ID NO: 81 - DNA, 20 bp, synthetic construct
  misc_feature 1..20: note = Description of Artificial Sequence: Synthetic oligonucleotide
  source 1..20: mol_type = other DNA
gaaggagaccgtctggcatc

SEQ ID NO: 82 - DNA, 20 bp, synthetic construct
  misc_feature 1..20: note = Description of Artificial Sequence: Synthetic oligonucleotide
  source 1..20: mol_type = other DNA
tcaaacataaactcccctgt

SEQ ID NO: 83 - DNA, 20 bp, synthetic construct
  misc_feature 1..20: note = Description of Artificial Sequence: Synthetic oligonucleotide
  source 1..20: mol_type = other DNA
aatctgttctgggcaggaag

SEQ ID NO: 84 - DNA, 20 bp, synthetic construct
  misc_feature 1..20: note = Description of Artificial Sequence: Synthetic oligonucleotide
  source 1..20: mol_type = other DNA
ccctgcagtcatagaagtcc

SEQ ID NO: 85 - DNA, 20 bp, synthetic construct
  misc_feature 1..20: note = Description of Artificial Sequence: Synthetic oligonucleotide
  source 1..20: mol_type = other DNA
tgtggaggtgaagacattgt

SEQ ID NO: 86 - DNA, 20 bp, synthetic construct
  misc_feature 1..20: note = Description of Artificial Sequence: Synthetic oligonucleotide
  source 1..20: mol_type = other DNA
tcgctctgaccaccgtgatg

SEQ ID NO: 87 - DNA, 20 bp, synthetic construct
  misc_feature 1..20: note = Description of Artificial Sequence: Synthetic oligonucleotide
  source 1..20: mol_type = other DNA
tgtggaactgagagagccca

SEQ ID NO: 88 - DNA, 20 bp, synthetic construct
  misc_feature 1..20: note = Description of Artificial Sequence: Synthetic oligonucleotide
  source 1..20: mol_type = other DNA
ccagtacctccagaggtaac

SEQ ID NO: 89 - DNA, 20 bp, synthetic construct
  misc_feature 1..20: note = Description of Artificial Sequence: Synthetic oligonucleotide
  source 1..20: mol_type = other DNA
gatgagcgctcaggaatcat

SEQ ID NO: 90 - DNA, 20 bp, synthetic construct
  misc_feature 1..20: note = Description of Artificial Sequence: Synthetic oligonucleotide
  source 1..20: mol_type = other DNA
gccactgtccatgaccccgt

SEQ ID NO: 91 - DNA, 20 bp, synthetic construct
  misc_feature 1..20: note = Description of Artificial Sequence: Synthetic oligonucleotide
  source 1..20: mol_type = other DNA
gtggagaacatatttcctga

SEQ ID NO: 92 - DNA, 20 bp, synthetic construct
  misc_feature 1..20: note = Description of Artificial Sequence: Synthetic oligonucleotide
  source 1..20: mol_type = other DNA
tgggccatctcaatctgaac

SEQ ID NO: 93 - DNA, 20 bp, synthetic construct
  misc_feature 1..20: note = Description of Artificial Sequence: Synthetic oligonucleotide
  source 1..20: mol_type = other DNA
tgctggaacttgaaggcgag

SEQ ID NO: 94 - DNA, 20 bp, synthetic construct
  misc_feature 1..20: note = Description of Artificial Sequence: Synthetic oligonucleotide
  source 1..20: mol_type = other DNA
tcatccaggataagtacaca

SEQ ID NO: 95 - DNA, 20 bp, synthetic construct
  misc_feature 1..20: note = Description of Artificial Sequence: Synthetic oligonucleotide
  source 1..20: mol_type = other DNA
gatcaatgctcgggccaacg

SEQ ID NO: 96 - DNA, 20 bp, synthetic construct
  misc_feature 1..20: note = Description of Artificial Sequence: Synthetic oligonucleotide
  source 1..20: mol_type = other DNA
acgccactgcctgtcgctga

SEQ ID NO: 97 - DNA, 20 bp, synthetic construct
  misc_feature 1..20: note = Description of Artificial Sequence: Synthetic oligonucleotide
  source 1..20: mol_type = other DNA
tgaggaagcaaagtccccag

SEQ ID NO: 98 - DNA, 20 bp, synthetic construct
  misc_feature 1..20: note = Description of Artificial Sequence: Synthetic oligonucleotide
  source 1..20: mol_type = other DNA
agccgcgtccaccagcagca

SEQ ID NO: 99 - DNA, 20 bp, synthetic construct
  misc_feature 1..20: note = Description of Artificial Sequence: Synthetic oligonucleotide
  source 1..20: mol_type = other DNA
tcctgaaagggttgaactgt

SEQ ID NO: 100 - DNA, 20 bp, synthetic construct
  misc_feature 1..20: note = Description of Artificial Sequence: Synthetic oligonucleotide
  source 1..20: mol_type = other DNA
tttccggtccatgggcccca

SEQ ID NO: 101 - DNA, 20 bp, synthetic construct
  misc_feature 1..20: note = Description of Artificial Sequence: Synthetic oligonucleotide
  source 1..20: mol_type = other DNA
ctcggggtagcaacaaaagg

SEQ ID NO: 102 - DNA, 20 bp, synthetic construct
  misc_feature 1..20: note = Description of Artificial Sequence: Synthetic oligonucleotide
  source 1..20: mol_type = other DNA
gccatggtcagcaagactcg

SEQ ID NO: 103 - DNA, 20 bp, synthetic construct
  misc_feature 1..20: note = Description of Artificial Sequence: Synthetic oligonucleotide
  source 1..20: mol_type = other DNA
tggcaaagtctcgaacatct

SEQ ID NO: 104 - DNA, 20 bp, synthetic construct
  misc_feature 1..20: note = Description of Artificial Sequence: Synthetic oligonucleotide
  source 1..20: mol_type = other DNA
attcggggatgcttcgcaaa

SEQ ID NO: 105 - DNA, 20 bp, synthetic construct
  misc_feature 1..20: note = Description of Artificial Sequence: Synthetic oligonucleotide
  source 1..20: mol_type = other DNA
ctattatgaagaatcaaagc

SEQ ID NO: 106 - DNA, 20 bp, synthetic construct
  misc_feature 1..20: note = Description of Artificial Sequence: Synthetic oligonucleotide
  source 1..20: mol_type = other DNA
cagttttaaaagacaggaca

SEQ ID NO: 107 - DNA, 20 bp, synthetic construct
  misc_feature 1..20: note = Description of Artificial Sequence: Synthetic oligonucleotide
  source 1..20: mol_type = other DNA
cctgagcaagcacactgctg

SEQ ID NO: 108 - DNA, 20 bp, synthetic construct
  misc_feature 1..20: note = Description of Artificial Sequence: Synthetic oligonucleotide
  source 1..20: mol_type = other DNA
ctaggttcttcagggtggga

SEQ ID NO: 109 - DNA, 20 bp, synthetic construct
  misc_feature 1..20: note = Description of Artificial Sequence: Synthetic oligonucleotide
  source 1..20: mol_type = other DNA
gtcctgacaggggagaaaga

SEQ ID NO: 110 - DNA, 20 bp, synthetic construct
  misc_feature 1..20: note = Description of Artificial Sequence: Synthetic oligonucleotide
  source 1..20: mol_type = other DNA
ttaggttctctggagcccag

SEQ ID NO: 111 - DNA, 20 bp, synthetic construct
  misc_feature 1..20: note = Description of Artificial Sequence: Synthetic oligonucleotide
  source 1..20: mol_type = other DNA
gttaggttctctggagccca

SEQ ID NO: 112 - DNA, 20 bp, synthetic construct
  misc_feature 1..20: note = Description of Artificial Sequence: Synthetic oligonucleotide
  source 1..20: mol_type = other DNA
aaggatacttggactggccc

SEQ ID NO: 113 - DNA, 20 bp, synthetic construct
  misc_feature 1..20: note = Description of Artificial Sequence: Synthetic oligonucleotide
  source 1..20: mol_type = other DNA
tcgagctttgatgtcaggaa

SEQ ID NO: 114 - DNA, 20 bp, synthetic construct
  misc_feature 1..20: note = Description of Artificial Sequence: Synthetic oligonucleotide
  source 1..20: mol_type = other DNA
acaggctacctggtcctgga

SEQ ID NO: 115 - DNA, 20 bp, synthetic construct
  misc_feature 1..20: note = Description of Artificial Sequence: Synthetic oligonucleotide
  source 1..20: mol_type = other DNA
tctccccaagcccatcgtag

SEQ ID NO: 116 - DNA, 20 bp, synthetic construct
  misc_feature 1..20: note = Description of Artificial Sequence: Synthetic oligonucleotide
  source 1..20: mol_type = other DNA
actctcttcacagccgaaga

SEQ ID NO: 117 - DNA, 20 bp, synthetic construct
  misc_feature 1..20: note = Description of Artificial Sequence: Synthetic oligonucleotide
  source 1..20: mol_type = other DNA
tagccccctacggctacact

SEQ ID NO: 118 - DNA, 20 bp, synthetic construct
  misc_feature 1..20: note = Description of Artificial Sequence: Synthetic oligonucleotide
  source 1..20: mol_type = other DNA
aagatcctttctgggaaagt

SEQ ID NO: 119 - DNA, 20 bp, synthetic construct
  misc_feature 1..20: note = Description of Artificial Sequence: Synthetic oligonucleotide
  source 1..20: mol_type = other DNA
catggtgagcgtggactttc

SEQ ID NO: 120 - DNA, 20 bp, synthetic construct
  misc_feature 1..20: note = Description of Artificial Sequence: Synthetic oligonucleotide
  source 1..20: mol_type = other DNA
ttgcttgttcagagaacaat

SEQ ID NO: 121 - DNA, 20 bp, synthetic construct
  misc_feature 1..20: note = Description of Artificial Sequence: Synthetic oligonucleotide
  source 1..20: mol_type = other DNA
attgtgttacaagaaagcat

SEQ ID NO: 122 - DNA, 22 bp, synthetic construct
  misc_feature 1..22: note = Description of Artificial Sequence: Synthetic oligonucleotide
  source 1..22: mol_type = other DNA
ggtctccttaaacctgtcttgt

SEQ ID NO: 123 - DNA, 30 bp, synthetic construct
  misc_feature 1..30: note = Description of Artificial Sequence: Synthetic oligonucleotide
  source 1..30: mol_type = other DNA
ggaggtggaggctctggtggaggcggatca

SEQ ID NO: 124 - DNA, 24 bp, synthetic construct
  misc_feature 1..24: note = Description of Artificial Sequence: Synthetic oligonucleotide
  source 1..24: mol_type = other DNA
gcagaggctgcagccgctaaggcc

SEQ ID NO: 125 - DNA, 42 bp, synthetic construct
  misc_feature 1..42: note = Description of Artificial Sequence: Synthetic oligonucleotide
  source 1..42: mol_type = other DNA
gcagaggctgcagccgctaaggaggcagctgccgctaaggcc

SEQ ID NO: 126 - DNA, 30 bp, synthetic construct
  misc_feature 1..30: note = Description of Artificial Sequence: Synthetic oligonucleotide
  source 1..30: mol_type = other DNA
gcacctgctccagcgcccgcaccagctccc

SEQ ID NO: 127 - DNA, 21 bp, synthetic construct
  misc_feature 1..21: note = Description of Artificial Sequence: Synthetic oligonucleotide
  source 1..21: mol_type = other DNA
aacgatcctgagacttccaca

SEQ ID NO: 128 - DNA, 21 bp, synthetic construct
  misc_feature 1..21: note = Description of Artificial Sequence: Synthetic oligonucleotide
  source 1..21: mol_type = other DNA
tgcttaccaagctgtgattcc

SEQ ID NO: 129 - RNA, 20 nt, synthetic construct
  misc_feature 1..20: note = Description of Artificial Sequence: Synthetic oligonucleotide
  source 1..20: mol_type = other RNA
gtaacggcagacttctcctc

SEQ ID NO: 130 - RNA, 20 nt, synthetic construct
  misc_feature 1..20: note = Description of Artificial Sequence: Synthetic oligonucleotide
  source 1..20: mol_type = other RNA
gaggtgaacgtggatgaagt

SEQ ID NO: 131 - DNA, 20 bp, synthetic construct
  misc_feature 1..20: note = Description of Artificial Sequence: Synthetic oligonucleotide
  source 1..20: mol_type = other DNA
aggcctttaccgatgtgatg

SEQ ID NO: 132 - DNA, 20 bp, synthetic construct
  misc_feature 1..20: note = Description of Artificial Sequence: Synthetic oligonucleotide
  source 1..20: mol_type = other DNA
acggagtctcgctctgtcac

SEQ ID NO: 133 - DNA, 20 bp, synthetic construct
  misc_feature 1..20: note = Description of Artificial Sequence: Synthetic oligonucleotide
  source 1..20: mol_type = other DNA
caaactgcaaggctgcaata

SEQ ID NO: 134 - DNA, 20 bp, synthetic construct
  misc_feature 1..20: note = Description of Artificial Sequence: Synthetic oligonucleotide
  source 1..20: mol_type = other DNA
gacccaccatgtcaaagtcc

SEQ ID NO: 135 - DNA, 17 bp, synthetic construct
  misc_feature 1..17: note = Description of Artificial Sequence: Synthetic oligonucleotide
  source 1..17: mol_type = other DNA
gggtcttcgagaagacc

SEQ ID NO: 136 - DNA, 19 bp, synthetic construct
  misc_feature 1..19: note = Description of Artificial Sequence: Synthetic oligonucleotide
  source 1..19: mol_type = other DNA
ggtcttctaactcaaaact

SEQ ID NO: 137 - DNA, 20 bp, synthetic construct
  misc_feature 1..20: note = Description of Artificial Sequence: Synthetic oligonucleotide
  source 1..20: mol_type = other DNA
ggagtgcaatggcgcgatct

SEQ ID NO: 138 - DNA, 20 bp, synthetic construct
  misc_feature 1..20: note = Description of Artificial Sequence: Synthetic oligonucleotide
  source 1..20: mol_type = other DNA
gcgccattgcactccagcct

SEQ ID NO: 139 - DNA, 20 bp, synthetic construct
  misc_feature 1..20: note = Description of Artificial Sequence: Synthetic oligonucleotide
  source 1..20: mol_type = other DNA
ttatttagagctagtgtact

SEQ ID NO: 140 - DNA, 20 bp, synthetic construct
  misc_feature 1..20: note = Description of Artificial Sequence: Synthetic oligonucleotide
  source 1..20: mol_type = other DNA
ggcatccaccctaggtacaa

SEQ ID NO: 141 - DNA, 20 bp, synthetic construct
  misc_feature 1..20: note = Description of Artificial Sequence: Synthetic oligonucleotide
  source 1..20: mol_type = other DNA
cgaggcagtagaatcgcttg

SEQ ID NO: 142 - DNA, 20 bp, synthetic construct
  misc_feature 1..20: note = Description of Artificial Sequence: Synthetic oligonucleotide
  source 1..20: mol_type = other DNA
cactaaggcgcagaagaagg

SEQ ID NO: 143 - DNA, 19 bp, synthetic construct
  misc_feature 1..19: note = Description of Artificial Sequence: Synthetic oligonucleotide
  source 1..19: mol_type = other DNA
gagccgagatcgcgccatg

SEQ ID NO: 144 - DNA, 20 bp, synthetic construct
  misc_feature 1..20: note = Description of Artificial Sequence: Synthetic oligonucleotide
  source 1..20: mol_type = other DNA
acacggtgaaaccctgtctc

SEQ ID NO: 145 - DNA, 19 bp, synthetic construct
  misc_feature 1..19: note = Description of Artificial Sequence: Synthetic oligonucleotide
  source 1..19: mol_type = other DNA
acagatggaaggcctcctg

SEQ ID NO: 146 - DNA, 20 bp, synthetic construct
  misc_feature 1..20: note = Description of Artificial Sequence: Synthetic oligonucleotide
  source 1..20: mol_type = other DNA
cgggactatggttgctgact

SEQ ID NO: 147 - DNA, 23 bp, synthetic construct
  misc_feature 1..23: note = Description of Artificial Sequence: Synthetic oligonucleotide
  source 1..23: mol_type = other DNA
cccataattgataagccaaaaca

SEQ ID NO: 148 - DNA, 5238 bp, synthetic construct
  misc_feature 1..5238: note = Description of Artificial Sequence: Synthetic polynucleotide
  source 1..5238: mol_type = other DNA
atgggtatccagggtctgctgcagttcatcaaagaagcttctgaaccgatccacgttcgtaaatacaaaggtcaggttgttgctgttgacacctactgctggctgcacaaaggtgctatcgcttgcgctgaaaaactggctaaaggtgaaccgaccgaccgttacgtaggcttctgcatgaaatttgttaacatgctgctgtctcacggtatcaaaccgatcctggttttcgacggttgcaccctgccgtctaaaaaagaagttgaacgttctcgtcgtgaacgtcgtcaggctaacctgctgaaaggtaaacagctgctgcgtgaaggtaaagtttctgaagctcgtgaatgcttcacccgttctatcaacatcacccacgctatggctcacaaagttatcaaagctgctcgttctcagggtgttgactgcctggttgctccgtacgaagctgacgctcagctggcttacctgaacaaagctggtatcgttcaggctatcatcaccgaagactctgacctgctggctttcggttgcaaaaaagttatcctgaaaatggaccagttcggtaacggtctggaaatcgaccaggctcgtctgggtatgtgccgtcagctcggcgacgtcttcaccgaagaaaaattccgttacatgtgcatcctgtctggttgcgactacctgtcttctctgcgtggtatcggtctggctaaagcttgcaaagttctgcgtctggctaacaacccggacatcgttaaagttatcaaaaaaatcggtcactacctgaaaatgaacatcaccgttccggaagactacatcaacggtttcatccgtgctaacaacaccttcctgtaccagctggttttcgacccgatcaaacgtaaactgatcccgctgaacgcttacgaagacgacgttgacccggaaaccctgtcttacgctggtcagtacgttgacgactctatcgctctgcagatcgctctgggtaacaaagacatcaacaccttcgaacagatcgacgactacaacccggacaccggctccggctccggctccggctccggctccgctatgccggctcactctcgtgataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgaccccaagaagaagaggaaggtgatggataagcatcaccaccaccatcactaa

SEQ ID NO: 149 - DNA, 1452 bp, synthetic construct
  misc_feature 1..1452: note = Description of Artificial Sequence: Synthetic polynucleotide
  source 1..1452: mol_type = other DNA
cactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaacgcgggaggcggagcttgcagtgagccgagatcgcgccatg

SEQ ID NO: 150 - DNA, 2231 bp, synthetic construct
  misc_feature 1..2231: note = Description of Artificial Sequence: Synthetic polynucleotide
  source 1..2231: mol_type = other DNA
ccatagacggagcaggacattcccgaaagtaagaggaggaaggcatccaccctaggtacaatacttgtatatatggggagatgtgctctgctacaagtttgtgataaaggattaattttcttagttactatattttgcaagaatcaacattattatctttaaacaaaattaagaatgcctttgttctccagatatagggatatctggacactcctaagtctgagtctgtttagtaaacattatttatttgttcccttaaccgtaaacatctagaagctaggaatgactgactttctgggaatgcagcccagaaagtctcagcctcattttcctagccctcactcaaaatggagttactctggttcaagtaactctgacacttttcttctctttttttcttcttttttccttcctttattttttattttttatttttgaaataagaaatcaagaatacttgatgtttcatctaaaacaatacccataattgataagccaaaacaaaaacctaggtcttctaactcaaaactaggatgttttgctgtctctgctgatactcggctgatcgttaataggtaattaacaaacaagccttgctatgtccccctcagtttattaccattagatcatatgcctactgtcaatcatattaatccacaactatgcatttcacaaaacttgccataaaaattcacaggtttcccgcttccctcgagttttcatttccgaagggtcccatgtaatataaaacttatattaaatacatttgtatgcttttctcttgctaatctttttttttgttttttgagactgagccttgctctgtcacccaggctggagtgggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtccaatggcgcgatctcggctcactgcaacctccgcttcccaggttcaagcgattctactgcctcgccctcccgagtagctgggaccacagatacgtgccacc

SEQ ID NO: 151 - DNA, 120 bp, synthetic construct
  misc_feature 1..120: note = Description of Artificial Sequence: Synthetic polynucleotide
  source 1..120: mol_type = other DNA
aacctcaaacagacaccatggtgcatctgactcctgtggagaattctgcagttactgcactgtggggcaaggtgaacgtggaagaggttggtggtgaggccctgggcaggttggtatcaa

SEQ ID NO: 152 - DNA, 120 bp, synthetic construct
  misc_feature 1..120: note = Description of Artificial Sequence: Synthetic polynucleotide
  source 1..120: mol_type = other DNA
ctgactcctgtggagaattctgcagttactgcactgtggggcaaggtgaacgtggaagaggttggtggtgaggccctgggcaggttggtatcaaggttacaagacaggtttaaggagacc

SEQ ID NO: 153 - DNA, 717 bp, synthetic construct
  misc_feature 1..717: note = Description of Artificial Sequence: Synthetic polynucleotide
  source 1..717: mol_type = other DNA
ctgcaataggaagctatcctattggtcaattatgtttggtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtggacttt

SEQ ID NO: 154 - DNA, 708 bp, synthetic construct
  misc_feature 1..708: note = Description of Artificial Sequence: Synthetic polynucleotide
  source 1..708: mol_type = other DNA
caataggaagctatcctattggtcaattatgtttggtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtg

SEQ ID NO: 155 - DNA, 683 bp, synthetic construct
  misc_feature 1..683: note = Description of Artificial Sequence: Synthetic polynucleotide
  source 1..683: mol_type = other DNA
gtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgangaggataacatggcctctctcccagcgacacatgagttacacatctttggctccatcaacggtgtggactttgaca

SEQ ID NO: 156 - DNA, 417 bp, synthetic construct
  misc_feature 1..417: note = Description of Artificial Sequence: Synthetic polynucleotide
  source 1..417: mol_type = other DNA
ccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaac

SEQ ID NO: 157 - DNA, 481 bp, synthetic construct
  misc_feature 1..481: note = Description of Artificial Sequence: Synthetic polynucleotide
  source 1..481: mol_type = other DNA
ccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaacgcaggaggcggagcttgcagtgagccgagatcgcgccactgcactccagcctgggtgacagagc

SEQ ID NO: 158 - DNA, 394 bp, synthetic construct
  misc_feature 1..394: note = Description of Artificial Sequence: Synthetic polynucleotide
  source 1..394: mol_type = other DNA
agtgcttatgtagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtantcccagctactcgggaggctgaggcaggagaatggcgtgaacgcatgaggcgga

SEQ ID NO: 159 - DNA, 500 bp, synthetic construct
  misc_feature 1..500: note = Description of Artificial Sequence: Synthetic polynucleotide
  source 1..500: mol_type = other DNA
aggcctttaccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaacgcgggaggcggagcttgcagtgagccgagatcgcgccatggcactccagcctgggtgacagagcgagactccgt

SEQ ID NO: 160 - DNA, 742 bp, synthetic construct
  misc_feature 1..742: note = Description of Artificial Sequence: Synthetic polynucleotide
  source 1..742: mol_type = other DNA
caaactgcaaggctgcaataggaagctatcctattggtcaattatgtttcgtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtggactttgacatggtgggtc

SEQ ID NO: 161 - DNA, 132 bp, synthetic construct
  misc_feature 1..132: note = Description of Artificial Sequence: Synthetic polynucleotide
  source 1..132: mol_type = other DNA
caaacagacaccatggtgcacctgactcctgaggagaagtctgccgttactgccctgtggggcaaggtgaacgtggatgaagttggtggtgaggccctgggcaggttggtatcaaggttacaagacaggttt

SEQ ID NO: 162 - DNA, 71 bp, Homo sapiens
  source 1..71: mol_type = genomic DNA
ctgactcctgaggagaagtctgccgttactgccctgtggggcaaggtgaacgtggatgaagttggtggtga

SEQ ID NO: 163 - DNA, 71 bp, synthetic construct
  misc_feature 1..71: note = Description of Artificial Sequence: Synthetic oligonucleotide
  source 1..71: mol_type = other DNA
ctgactcctgtggagaattctgcagttactgcactgtggggcaaggtgaacgtggaagaggttggtggtga

SEQ ID NO: 164 - DNA, 132 bp, synthetic construct
  misc_feature 1..132: note = Description of Artificial Sequence: Synthetic polynucleotide
  source 1..132: mol_type = other DNA
tagtagcttttctattctgtttaggaatagcaatgcctgaaccctctaagtctgctccagcccctaaaaagggttctaagaaggctatcactaaggcgcagaagaaggatggtaagaagcgtaagcgcagcc

SEQ ID NO: 165 - DNA, 130 bp, synthetic construct
  misc_feature 1..130: note = Description of Artificial Sequence: Synthetic polynucleotide
  source 1..130: mol_type = other DNA
gacggctgtgcgcctgctgctgcctggggagctggctaagcatgctgtgtcggagggcactaaagcagttaccaagtacactagctctaaagtgagcaagggcgaggaggataacatggcctctctccca

SEQ ID NO: 166 - DNA, 129 bp, synthetic construct
  misc_feature 1..129: note = Description of Artificial Sequence: Synthetic polynucleotide
  source 1..129: mol_type = other DNA
ntttggcctttnccgatgtgatgggcatggacgagctgtacaagtaagtgcttatgtaagcacttccaaacccaaaggctcttttcagagccacctactttgtcacaaggagagctataaccacaattt

SEQ ID NO: 167 - DNA, 131 bp, synthetic construct
  misc_feature 1..131: note = Description of Artificial Sequence: Synthetic polynucleotide
  source 1..131: mol_type = other DNA
tgtagtcccagctactcgggaggctgaggcaggagaatggcgtgaacgcaggaggcggagcttgcagtgagccgagatcgcgccactgcactccagcctgggtgacagagcgaagaactccntaaanntan

(19) United States
(12) Patent Application Publication
(10) Pub. No.: US 2024/0093207 A1
(43) Pub. Date: Mar. 21, 2024

(54) NON-TOXIC CAS9 ENZYME AND APPLICATION THEREOF

(71) Applicant: CRISP-HR Therapeutics, Inc., San Carlos, CA (US)

(72) Inventor: Christopher Hackley, San Carlos, CA (US)

(21) Appl. No.: 18/495,420

(22) Filed: Oct. 26, 2023

Related U.S. Application Data
(63) Continuation of application No. 17/368,369, filed on Jul. 6, 2021.
Application No. 17/368,369 is a continuation of application No. PCT/US2020/012438, filed on Jan. 6, 2020.
(60) Provisional application No. 62/789,347, filed on Jan. 7, 2019.
(60) Provisional application No. 62/823,477, filed on Mar. 25, 2019.
(60) Provisional application No. 62/824,164, filed on Mar. 26, 2019.
(60) Provisional application No. 62/855,612, filed on May 31, 2019.

(51) Int. Cl. C12N 15/62; C12N 9/22; C12N 15/11

(52) U.S. Cl. CPC C12N 15/62; C12N 9/22; C12N 15/111; C12N 2310/20; C12N 2320/31

(57) ABSTRACT

Compositions related to engineered Cas9 enzyme in reducing cellular toxicity and methods using thereof related to the selective targeting and editing endogenous nucleic acid segment in both normal cell and in cell associated with genetic diseases are disclosed. In some cases, a polypeptide comprising a human Exo1 enzyme or a first functional fragment thereof and a Cas9 enzyme or a second functional fragment thereof, which are connected by a linker peptide, is disclosed. In some cases, a polynucleotide encoding the polypeptide and a guide RNA (gRNA) is disclosed. Further, methods for treating single gene disorders utilizing either the polypeptide or the polynucleotide are disclosed.

### CROSS-REFERENCE

This application is a continuation application of U.S. Non-Provisional application Ser. No. 17/368,369, filed Jul. 6, 2021, which is a continuation application of International Application No. PCT/US2020/012438, filed Jan. 6, 2020, which claims priority to U.S. provisional application 62/789,347, filed on Jan. 7, 2019; U.S. provisional application 62/823,477, filed on Mar. 25, 2019; U.S. provisional application 62/824,164, filed on Mar. 26, 2019, and U.S. provisional application 62/855,612, filed on May 31, 2019, the entirety of which are hereby incorporated by reference herein.

### SEQUENCE LISTING

The instant application contains a Sequence Listing which has been submitted electronically in XML format and is hereby incorporated by reference in its entirety. Said XML file, created on Oct. 25, 2023, is named 55190-701.303 SL.xml and is 245,571 bytes in size.

### BACKGROUND

Targeted editing of nucleic acids is a highly promising approach for studying genetic functions and for treating and ameliorating symptoms of genetic disorders and diseases. Most notable target-specific genetic modification methods involve engineering and using of zinc finger nucleases (ZFNs), transcription activator like effector nucleases (TALENs), and RNA-guided DNA endonuclease Cas. Frequency of introducing mutations such as deletions and insertions at the targeted nucleic acids through the non-homologous end joining (NHEJ) repair mechanism limits the applications of genetic targeting and editing in the development of therapeutics.

### SUMMARY

The disclosure is summarized here in part in the claims disclosed herein. Disclosed herein is a method comprising introducing a first vector into a plurality of cells wherein said first vector encodes a fusion protein complex comprising a Cas9 nuclease fused to an exonuclease; wherein a viability of said plurality of cells comprising said vector is at least 1.5 times that of a second plurality of cells comprising a second vector encoding a Cas9 nuclease; wherein said second plurality of cells are K562 cells transfected with said second vector. The first vector can encode the Cas9 fused to an exonuclease and a gRNA. The exonuclease can be selected from the group consisting of MRE11, EXO1, EXOIII, EXOVII, EXOT, DNA2, CtIP, TREX1, TREX2, Apollo, RecE, RecJ, T5, Lexo, RecBCD, and Mungbean. A donor polynucleotide can be introduced into the first plurality of cells. The method can comprise making an edit to an abnormal locus of a gene by said Cas9-fused to an exonuclease. The donor polynucleotide can comprise an integration cassette further comprising a functional locus of said gene. The viability can be measured by resazurin assay. The exonuclease can be Exo1. The abnormal locus can be an abnormal locus of a HBB gene. The donor polynucleotide can encode a functional locus of said HBB gene. The fusion protein complex can encode at least one nuclear localization signal (NLS). The first vector encoding the fusion protein complex can have at least 80% sequence identity with any one of SEQ ID NO: 2-18. The first vector can be delivered by electroporation. The donor polynucleotide can comprise a mutated protospacer adjacent motif (PAM) sequence located at the immediate 3′ end of a cleavage site, wherein said mutated PAM sequence comprises 5′-NCG-3′ or 5′-NGC-3′. The fusion protein complex can be unable to cleave said mutated PAM sequence. The donor polynucleotide can be single-stranded DNA. The donor polynucleotide can be double-stranded DNA.

Disclosed herein is a polypeptide, comprising a first functional fragment, a second functional fragment comprising a Cas nuclease, and a linker peptide, wherein said first functional fragment is coupled to a first end of the linker peptide and the second functional fragment is coupled to a second end of said linker peptide; and when a first complex comprising said polypeptide and a ribonucleic acid (RNA) molecule is administered to a first plurality of cells, a reduced toxicity is observed in said first plurality of cells compared to said toxicity observed in a second plurality of cells when a second complex comprising a Cas9 nuclease and said RNA molecule is administered to said second plurality of cells. The first functional fragment can comprise an exonuclease wherein the exonuclease is selected from the group consisting of MRE11, EXO1, EXOIII, EXOVII, EXOT, DNA2, CtIP, TREX1, TREX2, Apollo, RecE, RecJ, T5, Lexo, RecBCD, and Mungbean. The RNA molecule can be a guide RNA. The exonuclease can be a human Exo1 enzyme. The N-terminal of the human Exo1 enzyme can be coupled to said C-terminal of said linker which is coupled to said C-terminal of said Cas nuclease. The human Exo1 enzyme can comprise SEQ ID NO: 1. The human Exo1 enzyme can comprise a fragment that has a 80% sequence identity of SEQ ID NO:1. The human Exo1 enzyme can comprise a fragment that has a 90% sequence identity of SEQ ID NO:1. The human Exo1 enzyme can comprise a fragment that has a 95% sequence identity of SEQ ID NO:1. The second functional fragment can comprise a Cas9 enzyme. The Cas9 enzyme can comprise a N-terminal nuclear localizing sequence (NLS) and a C-terminal NLS. The Cas9 enzyme can comprise a N-terminal nuclear localizing sequence (NLS). The Cas9 enzyme can comprise a C-terminal nuclear localizing sequence (NLS). The linker peptide can be selected from a group consisting of FL2X, SLA2X, AP5X, FL1X, SLA1X. The linker peptide can be SLA2X. The peptide can comprise 5 to 200 amino acids. The reduced toxicity can be quantified by measuring resorufin accumulation. After administration of said first complex, the first plurality of cells can have at least two times a number of viable cells compared to said second plurality of cells after administration of said second complex wherein the number of viable cells is quantified by a resorufin assay. After administration of the first complex, the first plurality of cells has at least two times said amount of HDR edited cells when compared to the second plurality of cells after administration of the second complex as quantified by a cellular HDR assay. The cellular HDR assay can comprise IHC, qPCR or deep sequencing.

Disclosed herein is a polynucleotide encoding the aforementioned polypeptide and the RNA molecule. The first end of the linker peptide can be a 3′ end and the second end of the linker peptide can be a 5′ end. The first end of said linker peptide can be a 5′end and the second end of said linker peptide can be a 3′ end. The RNA molecule can be a guide RNA (gRNA). The polynucleotide can comprise a homology directed repair (HDR) template. The gRNA can be selected from sequences listed in Table 2. The HDR template can be single-strand DNA. The HDR template can be double-strand DNA. The polynucleotide can be formulated in a liposome. The liposome can comprise a polyethylene glycol (PEG), a cell-penetrating peptide, a ligand, an aptamer, an antibody, or a combination thereof.

Disclosed herein is a vector comprising a nucleotide sequence of the aforementioned polypeptide. The vector can comprise a promoter. The promoter can be a CMV or a CAG promoter. The vector can be selected from a group consisting of retroviral vectors, adenoviral vectors, lentiviral vectors, herpesvirus vectors, and adeno-associated viral vectors. The vector can be an adeno-associated viral vector. Disclosed herein is a virus-like particle (VLP) comprising the aforementioned vector. Disclosed herein is a kit comprising the aforementioned polypeptide formulated in a compatible pharmaceutical excipient, an insert with administering instructions, reagents.

Disclosed herein is a kit comprising the aforementioned polynucleotide formulated in a compatible pharmaceutical excipient, an insert with administering instructions, reagents.

Disclosed herein is a kit comprising the aforementioned vector formulated in a compatible pharmaceutical excipient, an insert with administering instructions, reagents.

Disclosed herein is a method for inducing homologous recombination of DNA in a cell, comprising contacting the DNA with the aforementioned polypeptide.

Disclosed herein is a method for inducing HDR in a cell in vitro or ex vivo, comprising delivering the aforementioned polynucleotide into a cell. The cell can be a human cell, a non-human mammalian cell, a stem cell, a non-mammalian cell, an invertebrate cell, a plant cell, or a single-eukaryotic organism.

Disclosed herein is a method, comprising: contacting a first of plurality of cells with an aforementioned polynucleotide and a second plurality of cells with a second polynucleotide encoding a wild-type Cas9 enzyme; and inducing a site-specific cleavage at an intended locus followed by HDR in the first plurality of cells and the second plurality of cells; and recovering at least 30-90% more cells in the first plurality of cells compared to the second plurality of cells. The method can further comprise measuring cell viability by measuring an amount of resorufin produced in the first plurality of cells and the second plurality of cells. The first plurality of cells can have 2-5 times an amount of viable cells as quantified by a resorufin assay when compared to the second plurality of cells. The first plurality of cells and the second plurality of cells can comprise a human cell, a non-human mammalian cell, a stem cell, a non-mammalian cell, a invertebrate cell, a plant cell, or a single-eukaryotic organism. The human cell can be a T cell, a B cell, a dendritic cell, a natural killer cell, a macrophage, a neutrophil, an eosinophil, a basophil, a mast cell, a hematopoietic progenitor cell, a hematopoietic stem cell (HSC), a red blood cell, a blood stem cell, an endoderm stem cell, an endoderm progenitor cell, an endoderm precursor cell, a differentiated endoderm cell, a mesenchymal stem cell (MSC), a mesenchymal progenitor cell, a mesenchymal precursor cell, or a differentiated mesenchymal cell. The differentiated endoderm cell can be a hepatocytes progenitor cell, a pancreatic progenitor cell, a lung progenitor cell, or a tracheae progenitor cell. The differentiated mesenchymal cell can be a bone cell, a cartilage cell, a muscle cell, an adipose cell, a stromal cell, a fibroblast, or a dermal cell.

Disclosed herein is a method for treating a single gene disorder in a subject, comprising: culturing a plurality of primary cells obtained from said subject; administering the aforementioned polynucleotide to a plurality of primary cells, wherein the gRNA is configured to recognize a locus of the gene that causes said disorder and the HDR template is configured to provide a functioning sequence of the gene; and inducing a site-specific cleavage at the locus followed by HDR, wherein the functioning sequence of said gene is inserted at the locus. The method can further comprise selecting primary cells in which said functioning sequence of the gene is inserted at the locus; and reintroducing the selected primary cells back into the subject. The subject can be a mammal. The mammal can be a human. The plurality of primary cells can be selected from a group comprising T cells, B cells, dendritic cells, natural killer cells, natural killer cells, macrophages, neutrophils, eosinophils, basophils, mast cells, hematopoietic progenitor cells, hematopoietic stem cells (HSCs), red blood cells, blood stem cells, endoderm stem cells, endoderm progenitor cells, endoderm precursor cells, differentiated endoderm cells, mesenchymal stem cells (MSCs), mesenchymal progenitor cells, mesenchymal precursor cells, differentiated mesenchymal cells, hepatocytes progenitor cells, pancreatic progenitor cells, lung progenitor cells, tracheae progenitor cells, bone cells, cartilage cells, muscle cells, adipose cells, stromal cells, fibroblasts, and dermal cells. The gene that causes said single gene disorder can be selected from Table 3.

Disclosed herein is a method for treating sickle cell anemia caused by an abnormal HBB gene in a subject, comprising: culturing a plurality of primary cells obtained from said subject; administering the aforementioned polynucleotide to the plurality of primary cells, wherein the gRNA is configured to recognize a locus of said HBB gene that causes the disorder and the HDR template is configured to provide a functioning sequence of said HBB gene; and inducing a site-specific cleavage at said locus followed by HDR, wherein the functioning sequence of said HBB gene is inserted at the locus. The method can further comprise selecting primary cells in which said functioning sequence of said HBB gene is inserted at said locus; and reintroducing said selected primary cells back into said subject. The subject can be a mammal. The mammal can be a human. The primary cell can be a hematopoietic stem cell. The primary cell can be a CD34+ hematopoietic stem cell. The primary cell can be a CD34+ hematopoietic stem cell. The vector can comprise plasmid PX330. The cell can be a CD34+ hematopoietic stem cell.

Disclosed herein is a method for treating sickle cell anemia caused by an abnormal HBB gene in a subject, comprising: culturing a plurality of primary cells obtained from the subject; administering the aforementioned polynucleotide to the plurality of primary cells, wherein the gRNA is configured to recognize a locus of the HBB gene that causes the disorder and the HDR template is configured to provide a functioning sequence of the HBB gene; and inducing a site-specific cleavage at the locus followed by HDR, wherein the functioning sequence of the HBB gene is inserted at the locus. The method can further comprise selecting primary cells in which the functioning sequence of the HBB gene is inserted at the locus; and reintroducing the selected primary cells back into the subject. The subject can be a mammal. The mammal can be a human. The primary cell can be a CD34+ hematopoietic stem cell.

Disclosed herein is a method, comprising: contacting a first of plurality of cells with a first complex comprising the aforementioned polynucleotide and a RNA molecule; inducing a site-specific cleavage followed by HDR in the first plurality of cells, wherein a percentage of cells of the first plurality of cells edited by HDR quantified by a cellular HDR assay is at least two times higher compared to a percentage of cells of a second plurality of cells contacted with a second complex comprising a polynucleotide encoding a wild-type Cas9 enzyme and the RNA molecule. The cellular HDR assay can comprise IHC. The cellular HDR assay can comprise qPCR. The cellular HDR assay can comprise nucleic acid sequencing.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Some understanding of the features and advantages of the present disclosure will be obtained by reference to the following detailed description that sets forth illustrative embodiments, in which the principles of the disclosure are utilized, and the accompanying drawings of which:

FIG. 1 shows embodiments of fusion proteins comprising hExo1enzyme and Cas9 enzyme linked together through different linkers.

FIG. 2 shows an embodiment of an intended target site and a HDR template.

FIG. 3 shows an embodiment of conducting a resazurin reduction assay. Column1-8 correspond to Cas9-HR fusion proteins 1-8 described in FIG. 1 respectively.

FIG. 4 shows a normalized fold change of resorufin fluorescence of cells transfected with RNP plasmids, GFP plasmids, and control plasmids before puromycin selection.

FIG. 5 shows a normalized fold change of resorufin fluorescence of cells transfected with wild type Cas9 enzyme plasmids treated with either dimethyl sulfoxide (DMSO) or pifithrin-α (PFT-α).

FIG. 6A shows an embodiment of an intended target site with three gRNA sequences (G1, G2, and G3; SEQ ID NO: 21, SEQ ID NO: 22, SEQ ID NO: 23 respectively) designed to target Exon 1 of the HBB gene.

FIG. 6B shows a normalized fold change of resorufin fluorescence of cells transfected with RNP plasmids with three gRNA sequences (G1, G2, and G3; SEQ ID NO: 21, SEQ ID NO: 22, SEQ ID NO: 23 respectively) designed to target Exon 1 of the HBB gene.

FIG. 6C shows a Cas9 HBB-G3 reverse Sanger sequence trace (SEQ ID NO: 161).

FIG. 7 shows an embodiment of conducting a resazurin reduction assay. Column 1-9 correspond to Cas9-HR fusion proteins 1-9 of fusion proteins described in FIG. 1 respectively.

FIG. 8 shows a normalized fold change of resorufin fluorescence of cells transfected with RNP plasmids with different gRNA sequences, GFP plasmids, and two different control plasmids to control cells.

FIGS. 9A-B show a normalized fold change of resorufin fluorescence of cells transfected with RNP plasmids. FIG. 9A shows the G2 (SEQ ID NO: 22) and G3 (SEQ ID NO: 23) gRNA targeting the exon 1 of the HBB gene. FIG. 9B shows that RNP plasmids with the seventh fusion protein (FIGS. 1) and G3 gRNA have less cellular toxicity compared to RNP plasmids with the unmodified Cas9 and G2 gRNA.

FIG. 10 shows a normalized fold change of resorufin fluorescence of cells transfected with different RNP plasmids targeting exon 1 of HBB gene.

FIG. 11A is a diagram of Plasmid PX330 which contains a constitutive promoter for mammalian Cas9 expression, along with U6 promoter driven gRNA expression.

FIG. 11B is an example of the experimental set up wherein cells are seeded and after two days of growth cellular toxicity is quantified.

FIG. 11C is a graph showing reduced cellular toxicity in A549 cells as shown in the FIG. 11B experimental set up and a diagram of the gRNA targeting intergenic region on Chromosome 12 depicted above the graph.

FIG. 11D is a graph showing that treatment with alpha-pifithrin (10 micromolar) reduces Cas9 induced cellular toxicity in A549 cells.

FIG. 12A is a diagram of the Puromycin resistance repair template (RT).

FIG. 12B shows the method used to quantify HDR and INDEL rates of hExo-Cas9 fusions in A549 cells.

FIG. 12C is a graph depicting the toxicity of various constructs tested via a resazurin assay.

FIG. 12D depicts the method of the resazurin assay.

FIG. 12E is a depiction of the genomic region of cells successfully integrated by the Puro-RT.

FIG. 12F is a graph of the survival of K562 cells transfected with either Cas9-HR8 (8) or Cas9 (NT) with G2 or G3 RNA after three days of puromycin treatment.

FIG. 12G is an agarose gel of the amplification products of the primers depicted in FIG. 12E showing stable integration of the repair template using Cas9-HR8 (fusion protein 8 of FIG. 1) and Cas9 (NT) with gRNA G2 or G3 in the genome.

FIG. 13A shows the genomic region, including the first two exons of HBB targeted to edit the Human Hemoglobin Beta (HBB) gene and a graph depicting data from the toxicity screen of HBB gRNA guides in A549 cells.

FIG. 13B shows sanger sequencing of the HBB genomic region in the HBB-G3 treated A549 cells (SEQ ID NO: 161).

FIG. 13C is a diagram of the wild-type HBB sequence (SEQ ID NO: 162) and the SSRT-G3 sequence (SEQ ID NO: 163) which introduces the sickle cell (E6V) an missense mutation which results in an EcoRI site and four silent mismatch mutations (bolded nucleotides a, a, a, and g on single-stranded repair template, SSRT G3) with the HBB-G3 gRNA highlighted by the bar from above. Mutations are designed to prevent gRNA binding upon successful repair

FIG. 13D depicts a HBB editing experiment in which K562 cells or A549 cells are electroporated with Cas9+SSRT-G3, Cas9-HR 1-9+SSRT-G3 or SSRT-G3 alone.

FIG. 14 illustrates toxicity assessment of two transfection methods, lipofectamine and calcium phosphate (CalPhos) as determined by transfecting A549 cells with HBB-G3 gRNA and Cas9-HR fusion proteins 4 and 5 as depicted in FIG. 1.

FIG. 15 illustrates toxicity assessment by transfecting A549 cells with HBB repair templates of FIG. 13A. Resazurin levels are measured on day 2 after the transfection.

FIG. 16A shows an agarose gel of EcoRI digestion assay of Cas9-HR fusion protein 8 of FIG. 1 integrating the HBB repair template into the genome of K562 cells. Arrows indicate the EcoRI digested products. There are no EcoRI digested products in lanes of Cas9 only (NT), SSRT, and Con (no Cas9).

FIG. 16B shows an agarose gel of EcoRI digestion assay of Cas9-HR fusion proteins 4, 5, 6, 7, and 8 of FIG. 1 integrating the HBB repair template into the genome of K562 cells. Arrows indicate the EcoRI digested products. There are no EcoRI digested products in NT and Con lanes.

FIG. 16C shows a western blotting of Cas9-HR fusion proteins 4, 5, 6, 7, and 8 of FIG. 1, Cas9 only (NT), and Con (no Cas9). Arrow indicates detection of Cas9 in Cas9-HR fusion proteins and NT lanes.

FIG. 16D shows successful expression and purification in E. Coli of Cas9-HR 3. Successful expression and purification of Cas9 (lanes 8-14) is also shown to aid comparison.

FIG. 16E shows an immunohistochemistry (IHC) of the same transfected cells from FIG. 16C. Arrows indicates that Cas9-HR fusions and Cas9 are localized to the nucleus of the cells.

FIG. 17A illustrates the construct for a full H2B knock-in experiment.

FIG. 17B illustrates p53-depedent decrease of cellular toxicity induced by Cas-HR fusion proteins 4, 5, 6, and 8 of FIG. 1, Cas9 only (NT), and Con (no Cas9) in epithelial lung cancer cell lines. A549 cells are positive for p53 activity, while H1299 cells are negative for p53 activity. Toxicity as determined by normalized resazurin levels (y-axis) has shown that absence of p53 in H1299 cells yields lower cellular toxicity.

FIG. 17C illustrates the assessment of successful GFP tagging of H2B as diagrammed in FIG. 17A in K562 cells. Arrows indicate successful tagging of H2B with GFP as shown by detection of GFP in the nucleus.

FIG. 18A illustrates the schematic difference between Cas9 only model and Cas9-HR model. The presence of an Exonuclease domain fundamentally changes the predicted in-vitro cleavage pattern. Exo1 has a significant preference for phosphorylated 5′ termini vs non-phosphorylated. Therefore, it can be expected when using PCR products or other pieces of DNA lacking 5′-phosphorlyated termini that endonuclease cleavage via Cas9 can dominate initially, whereas after cleavage the two fragments each can possess 5′-phosphorlyated termini, which result in rapid degradation via the hExo1.

FIG. 18B illustrates an exemplary digestion pattern based on FIG. 18A. Only Cas9-HR3+ gRNA and Cas9-HR3 can produce the digested products which demonstrate successful in-vitro nuclease activity. Additionally, though hExo1strongly prefers phosphorylated 5′-termini, hExo1can still bind and resect unphosphorylated 5′-termini, so a small amount degradation without gRNAs when compared to Cas9.

FIG. 18C illustrates an actual agarose example of FIG. 18A and FIG. 18B. Lanes 1 and 2 show Cas9-HR3 targeting either HBB-G1 or HBB-G3, Lanes 3 and 4 show Cas9 (NT) targeting either HBB-G1 or HBB-G3, Lane 5 is untreated DNA.

FIG. 18D illustrates a similar experiment as FIG. 18C and differs only by conducting the experiment after leaving enzymes for 2 weeks at 4° C. in order to compare protein stability. Lane 1 is digestion pattern from the combination of Cas9-HR3 and gRNA HBB-G1. Lane 2 is digestion pattern from the combination of Cas9 and gRNA HBB-G1. Lane 3 is digestion pattern from the combination of Cas9-HR3 and HBB-G3. Lane 4 is digestion pattern from the combination of Cas9 and HBB-G3. Lane 5 is digestion pattern from Cas9-HR only. Lane 6 is digestion pattern from Cas9 only. Lane 7 is the control, where there is neither Cas9 nor gRNA.

FIGS. 19A-G illustrates induction of genomic integration of the H2BmNeon fusion via Cas9-HR 4, Cas9-HR 8, Cas9 only (NT) and Control without Cas9 (Con). FIG. 19A illustrates design of H2B integration detection primers. Two sets of primers are designed to bind outside of the 5′ and 3′ ends of the repair template annealing to sequences only present in the genome, not in the RT, while the others anneal to sequences specific to the repair template, and are not present in the unmodified cells. FIG. 19B illustrates an agarose gel showing PCR products amplified by the 5′ primers, indicating successful tagging of endogenous H2B with GFP. FIG. 19C illustrates an agarose gel showing PCR products amplified by the 3′ primers, indicating successful tagging of endogenous H2B with GFP. FIG. 19D illustrates absorbance of sequence trace from Sanger sequencing of the PCR product amplified by the 5′ primers. Figure discloses SEQ ID NOS 164-165, respectively, in order of appearance. FIG. 19E illustrates absorbance of sequence trace from Sanger sequencing of the PCR product amplified by the 3′ primers. Figure discloses SEQ ID NOS 166-167, respectively, in order of appearance. FIG. 19F illustrates sequencing alignment of the PCR product amplified by the 5′ primers and discloses SEQ ID NOS 155, 154, 153, and 160, respectively, in order of appearance. FIG. 19G illustrates sequencing alignment of the PCR product amplified by the 3′ primers and discloses SEQ ID NOS 158, 157, 156, and 159, respectively, in order of appearance.

FIG. 20 illustrates designs for additional Cas9-HR fusion proteins with expanded functionalities.

### DETAILED DESCRIPTION

A brief description about the CRISPR (Clustered Regularly Interspaced Short Palindromic Repeats)/Cas (CRISPR-associated) system is included. The CRISPR/Cas enzyme system first found in bacteria and archaea is an immune defense against viral infection. During viral infection, segments of viral DNA are integrated into CRISPR locus. These segments of integrated viral DNA are transcribed into guide RNA (gRNA), which is sequentially complementary to the viral genome. gRNA directs the Cas enzymes to the gRNA targeted viral genome, where Cas proteins cleave the viral genome, thus defending against viral infection.

The CRISPR system typically comprises a gRNA that is specific to the target DNA sequence and a non-specific Cas 9 protein. Generally, the gRNA includes two distinct segments-CRISPR RNA (crRNA) and transactivating CRISPR RNA (tracrRNA). The crRNA is complementary to the target DNA sequences, and thus recognize the sequence to be cleaved. And the tracrRNA functions as a scaffold for the crRNA-Cas9 interaction. Guide RNA naturally form a duplex molecule, with the crRNA and tracrRNA fragments annealed together. Cas proteins have been investigated and engineered as a tool for genetic editing by generating site-specific double strand breaks (DSBs). Custom designed gRNA directs the Cas proteins to generate DSB at any nucleic acid loci that are complementary to the sequence of gRNA. Cas proteins have been shown to successfully introduce nucleotide changes, deletions, insertions, and substitutions in eukaryotic cells.

The use CRISPR and Cas9 proteins for editing nucleic acids are limited by the endogenous repair mechanism of the cell. DSBs are preferentially repaired by NHEJ. Unintended insertions and deletions at sites of repair associated with NHEJ render development of genetic-based therapy undesirable. Alternatively, if the generated DSBs are resected so that long (<200 bp) 3′ overhangs are generated, the endogenous repair pathway is forced to use HR. Targeted error-free insertions and deletions of anywhere from 1-1000 s of bp of DNA can be achieved by addition of a polynucleotide (template sequence) comprising homology arms flanking the desired insertion or deletion.

Homology directed repair is error free, and results in the ability to insert or delete specific sequences of DNA in a given genome.

Further, the HDR reduces cellular toxicity, which is caused by DSBs introduced by CRISPR and Cas9 enzyme system. The cellular toxicity is dependent on the p53 tumor suppressor pathway, as inhibition or loss of p53 function greatly reduces cellular toxicity in both Human Pluripotent Stem Cells (hPSCs) and in immortalized Retinal Pigment Epithelium (RPE) cells. Since permanent loss of p53 functionality has some severe effects on cells including genomic instability, altered cellular homeostasis, and increased rates of cancer in-vivo, one solution is transient inhibition of p53 by either small molecule or overexpression of dominant negative inhibitors. However, the transient inhibition of p53 in vivo is challenging and could produce undesirable side effects. Therefore, generating a non-toxic Cas9 enzyme is desirable for in vivo applications.

Disclosed herein are compositions and methods related to the selective targeting and editing endogenous nucleic acid segment in both normal cell and in cell associated with genetic diseases with reduced cellular toxicity. Targeted endogenous nucleic acids are cleaved, digested, and edited through HDR. gRNA directs a protein fusion complex comprising of the Cas protein moiety and a human Exo1 enzyme to a specific endogenous nucleic acid segment, where the protein fusion complex introduces cleavage and digestion, leaving 3′ or 5′ overhangs on the targeted endogenous nucleic acid segment. The overhangs allow for increased rates of HDR when the cell is further presented with a polynucleotide fragment that shares some degrees of sequence homology as the targeted and digested endogenous nucleic acid segment.

Disclosed herein are compositions wherein the targeted endogenous nucleic acids are located in known disease loci. Targeted known disease loci are cleaved, digested, and edited through HDR. gRNA directs a protein fusion complex comprising the Cas protein moiety and a human Exo1 enzyme to a specific known disease locus where the protein fusion complex introduces cleavage and digestion, leaving 3′ or 5′ overhangs on the targeted endogenous nucleic acid segment. The overhangs allow for increased rates of HDR when the cell is further presented with a polynucleotide fragment that shares some degrees of sequence homology as the targeted and digested endogenous nucleic acid segment.

Fusion protein composition

Some aspects of the compositions and methods disclosed herein involve at least one modified polypeptide comprising a programmable endonuclease such as a Cas9 or other CRISPR-related programmable endonucleases coupled to a fragment of an exonuclease such as human Exo1 exonuclease or other exonucleases, such as MRE11, EXO1, EXOIII, EXOVII, EXOT, DNA2, CtIP, TREX1, TREX2, Apollo, RecE, RecJ, T5, Lexo, RecBCD, and Mungbean, to reduce cellular toxicity relative to that of an unmodified programmable endonuclease such as Cas9 enzyme in the CRISPR-Cas9 system.

### Cas9 Protein

The polypeptide (fusion protein) comprises a programmable endonuclease such as Cas9, other CRISPR-related programmable endonucleases, other site-specific endonucleases, or a fragment thereof and an exonuclease such as human Exo1 exonuclease or a fragment thereof covalently connected by a peptidyl linker. As used herein, the “Cas9,” “Cas9 domain,” or “Cas9 fragment” refers to an RNA-guided nuclease comprising a Cas9 protein, or a fragment thereof, e.g., a protein comprising an active DNA cleavage domain of Cas9. A Cas9 nuclease is sometimes referred to as a casnl nuclease or a CRISPR (clustered regularly interspaced short palindromic repeat)-associated nuclease. Cas9 nuclease sequences and structures are well known to those of ordinary skill in the art. Cas9 orthologs have been described in various species, including, but not limited to, S. pyogenes and S. thermophilus. Wild type (unmodified) Cas9 can be from any of the sequences listed below in Table 1. The Cas9 protein sequences listed in Table 1 is not meant to be limiting. Additional suitable Cas9 nucleases and protein sequences will be apparent to a person of ordinary skill in the art.

TABLE 1
Peptide sequences of various Cas9.

| | | |
|---|---|---|
| SEQ | NCBI Reference | MDKKYSIGLDIGTNSVGWAVITDDYKVPSKKFKVLGNTDR |
| ID | Sequence: | HSIKKNLIGALLFGSGETAEATRLKRTARRRYTRRKNRICY |
| NO: | NC_017053.1 | LQEIFSNEMAKVDDSFFHRLEESFLVEEDKKHERHPIFGNIV |
| 2 | (Streptococcus | DEVAYHEKYPTIYHLRKKLADSTDKADLRLIYLALAHMIK |
| | pyogenes) | FRGHFLIEGDLNPDNSDVDKLFIQLVQIYNQLFEENPINASR |
| | | VDAKAILSARLSKSRRLENLIAQLPGEKRNGLFGNLIALSLG |
| | | LTPNFKSNFDLAEDAKLQLSKDTYDDDLDNLLAQIGDQYA |
| | | DLFLAAKNLSDAILLSDILRVNSEITKAPLSASMIKRYDEHH |
| | | QDLTLLKALVRQQLPEKYKEIFFDQSKNGYAGYIDGGASQ |
| | | EEFYKFIKPILEKMDGTEELLVKLNREDLLRKQRTFDNGSIP |
| | | HQIHLGELHAILRRQEDFYPFLKDNREKIEKILTFRIPYYVGP |
| | | LARGNSRFAWMTRKSEETITPWNFEEVVDKGASAQSFIER |
| | | MTNFDKNLPNEKVLPKHSLLYEYFTVYNELTKVKYVTEG |
| | | MRKPAFLSGEQKKAIVDLLFKTNRKVTVKQLKEDYFKKIE |
| | | CFDSVEISGVEDRFNASLGAYHDLLKIIKDKDFLDNEENEDI |
| | | LEDIVLTLTLFEDRGMIEERLKTYAHLFDDKVMKQLKRRR |
| | | YTGWGRLSRKLINGIRDKQSGKTILDFLKSDGFANRNFMQL |
| | | IHDDSLTFKEDIQKAQVSGQGHSLHEQIANLAGSPAIKKGIL |
| | | QTVKIVDELVKVMGHKPENIVIEMARENQTTQKGQKNSRE |
| | | RMKRIEEGIKELGSQILKEHPVENTQLQNEKLYLYYLQNGR |
| | | DMYVDQELDINRLSDYDVDHIVPQSFIKDDSIDNKVLTRSD |
| | | KNRGKSDNVPSEEVVKKMKNYWRQLLNAKLITQRKFDNL |
| | | TKAERGGLSELDKAGFIKRQLVETRQITKHVAQILDSRMNT |
| | | KYDENDKLIREVKVITLKSKLVSDFRKDFQFYKVREINNYH |
| | | HAHDAYLNAVVGTALIKKYPKLESEFVYGDYKVYDVRKM |
| | | IAKSEQEIGKATAKYFFYSNIMNFFKTEITLANGEIRKRPLIE |
| | | TNGETGEIVWDKGRDFATVRKVLSMPQVNIVKKTEVQTG |
| | | GFSKESILPKRNSDKLIARKKDWDPKKYGGFDSPTVAYSVL |
| | | VVAKVEKGKSKKLKSVKELLGITIMERSSFEKNPIDFLEAK |
| | | GYKEVKKDLIIKLPKYSLFELENGRKRMLASAGELQKGNE |
| | | LALPSKYVNFLYLASHYEKLKGSPEDNEQKQLFVEQHKHY |
| | | LDEIIEQISEFSKRVILADANLDKVLSAYNKHRDKPIREQAE |
| | | NIIHLFTLTNLGAPAAFKYFDTTIDRKRYTSTKEVLDATLIH |
| | | QSITGLYETRIDLSQLGGD (single underline: HNH domain; |
| | | double underline: RuvC domain) |
| SEQ | Streptococcus | MLFNKCIIISINLDFSNKEKCMTKPYSIGLDIGTNSVGWAVIT |
| ID | thermophilus | DNYKVPSKKMKVLGNTS |
| NO: | | KKYIKKNLLGVLLFDSGITAEGRRLKRTARRRYTRRRNRIL |
| 3 | | YLQEIFSTEMATLDDAFFQ |
| | | RLDDSFLVPDDKRDSKYPIFGNLVEEKVYHDEFPTIYHLRK |
| | | YLADSTKKADLRLVYLALA |
| | | HMIKYRGHFLIEGEFNSKNNDIQKNFQDFLDTYNAIFESDLS |
| | | LENSKQLEEIVKDKISKL |
| | | EKKDRILKLFPGEKNSGIFSEFLKLIVGNQADFRKCFNLDEK |
| | | ASLHFSKESYDEDLETLL |
| | | GYIGDDYSDVFLKAKKLYDAILLSGFLTVTDNETEAPLSSA |
| | | MIKRYNEHKEDLALLKEYI |
| | | RNISLKTYNEVFKDDTKNGYAGYIDGKTNQEDFYVYLKNL |
| | | LAEFEGADYFLEKIDREDFL |
| | | RKQRTFDNGSIPYQIHLQEMRAILDKQAKFYPFLAKNKERI |
| | | EKILTFRIPYYVGPLARGN |
| | | SDFAWSIRKRNEKITPWNFEDVIDKESSAEAFINRMTSFDLY |
| | | LPEEKVLPKHSLLYETFN |
| | | VYNELTKVRFIAESMRDYQFLDSKQKKDIVRLYFKDKRKV |
| | | TDKDIIEYLHAIYGYDGIEL |
| | | KGIEKQFNSSLSTYHDLLNIINDKEFLDDSSNEAIIEEIIHTLTI |
| | | FEDREMIKQRLSKFE |
| | | NIFDKSVLKKLSRRHYTGWGKLSAKLINGIRDEKSGNTILD |
| | | YLIDDGISNRNFMQLIHDD |
| | | ALSFKKKIQKAQIIGDEDKGNIKEVVKSLPGSPAIKKGILQSI |
| | | KIVDELVKVMGGRKPES |
| | | IVVEMARENQYTNQGKSNSQQRLKRLEKSLKELGSKILKE |
| | | NIPAKLSKIDNNALQNDRLY |
| | | LYYLQNGKDMYTGDDLDIDRLSNYDIDHIIPQAFLKDNSID |
| | | NKVLVSSASNRGKSDDFPS |
| | | LEVVKKRKTFWYQLLKSKLISQRKFDNLTKAERGGLLPED |
| | | KAGFIQRQLVETRQITKHVA |
| | | RLLDEKFNNKKDENNRAVRTVKIITLKSTLVSQFRKDFELY |
| | | KVREINDFHHAHDAYLNAV |
| | | IASALLKKYPKLEPEFVYGDYPKYNSFRERKSATEKVYFYS |
| | | NIMNIFKKSISLADGRVIE |
| | | RPLIEVNEETGESVWNKESDLATVRRVLSYPQVNVVKKVE |
| | | EQNHGLDRGKPKGLFNANLS |
| | | SKPKPNSNENLVGAKEYLDPKKYGGYAGISNSFAVLVKGTI |
| | | EKGAKKKITNVLEFQGIST |
| | | LDRINYRKDKLNFLLEKGYKDIELIIELPKYSLFELSDGSRR |
| | | MLASILSTNNKRGEIHKG |
| | | NQIFLSQKFVKLLYHAKRISNTINENHRKYVENHKKEFEEL |
| | | FYYILEFNENYVGAKKNGK |
| | | LLNSAFQSWQNHSIDELCSSFIGPTGSERKGLFELTSRGSAA |
| | | DFEFLGVKIPRYRDYTPS |
| | | SLLKDATLIHQSVTGLYETRIDLAKLGEG |
| SEQ | Francisella | MNFKILPIAIDLGVKNTGVFSAFYQKGTSLERLDNKNGKVY |
| ID | tularensis | ELSKDSYTLLMNNRTARRH |
| NO: | subsp. | QRRGIDRKQLVKRLFKLIWTEQLNLEWDKDTQQAISFLFNR |
| 4 | novicida (strain | RGFSFITDGYSPEYLNIVP |
| | U112) | EQVKAILMDIFDDYNGEDDLDSYLKLATEQESKISEIYNKL |
| | | MQKILEFKLMKLCTDIKDD |
| | | KVSTKTLKEITSYEFELLADYLANYSESLKTQKFSYTDKQG |
| | | NLKELSYYHHDKYNIQEFL |
| | | KRHATINDRILDTLLTDDLDIWNFNFEKFDFDKNEEKLQNQ |
| | | EDKDHIQAHLHHFVFAVNK |
| | | IKSEMASGGRHRSQYFQEITNVLDENNHQEGYLKNFCENL |
| | | HNKKYSNLSVKNLVNLIGNL |
| | | SNLELKPLRKYFNDKIHAKADHWDEQKFTETYCHWILGE |
| | | WRVGVKDQDKKDGAKYSYKDL |
| | | CNELKQKVTKAGLVDFLLELDPCRTIPPYLDNNNRKPPKCQ |
| | | SLILNPKFLDNQYPNWQQY |
| | | LQELKKLQSIQNYLDSFETDLKVLKSSKDQPYFVEYKSSNQ |
| | | QIASGQRDYKDLDARILQF |
| | | IFDRVKASDELLLNEIYFQAKKLKQKASSELEKLESSKKLD |
| | | EVIANSQLSQILKSQHTNG |
| | | IFEQGTFLHLVCKYYKQRQRARDSRLYIMPEYRYDKKLHK |
| | | YNNTGRFDDDNQLLTYCNHK |
| | | PRQKRYQLLNDLAGVLQVSPNFLKDKIGSDDDLFISKWLV |
| | | EHIRGFKKACEDSLKIQKDN |
| | | RGLLNHKINIARNTKGKCEKEIFNLICKIEGSEDKKGNYKH |
| | | GLAYELGVLLFGEPNEASK |
| | | PEFDRKIKKFNSIYSFAQIQQIAFAERKGNANTCAVCSADN |
| | | AHRMQQIKITEPVEDNKDK |
| | | IILSAKAQRLPAIPTRIVDGAVKKMATILAKNIVDDNWQNIK |
| | | QVLSAKHQLHIPIITESN |
| | | AFEFEPALADVKGKSLKDRRKKALERISPENIFKDKNNRIK |
| | | EFAKGISAYSGANLTDGDF |
| | | DGAKEELDHIIPRSHKKYGTLNDEANLICVTRGDNKNKGN |
| | | RIFCLRDLADNYKLKQFETT |
| | | DDLEIEKKIADTIWDANKKDFKFGNYRSFINLTPQEQKAFR |
| | | HALFLADENPIKQAVIRAI |
| | | NNRNRTFVNGTQRYFAEVLANNIYLRAKKENLNTDKISFD |
| | | YFGIPTIGNGRGIAEIRQLY |
| | | EKVDSDIQAYAKGDKPQASYSHLIDAMLAFCIAADEHRND |
| | | GSIGLEIDKNYSLYPLDKNT |
| | | GEVFTKDIFSQIKITDNEFSDKKLVRKKAIEGFNTHRQMTR |
| | | DGIYAENYLPILIHKELNE |
| | | VRKGYTWKNSEEIKIFKGKKYDIQQLNNLVYCLKFVDKPIS |
| | | IDIQISTLEELRNILTTNN |
| | | IAATAEYYYINLKTQKLHEYYIENYNTALGYKKYSKEMEF |
| | | LRSLAYRSERVKIKSIDDVK |
| | | QVLDKDSNFIIGKITLPFKKEWQRLYREWQNTTIKDDYEFL |
| | | KSFFNVKSITKLHKKVRKD |
| | | FSLPISTNEGKFLVKRKTWDNNFIYQILNDSDSRADGTKPFI |
| | | PAFDISKNEIVEAIIDSF |
| | | TSKNIFWLPKNIELQKVDNKNIFAIDTSKWFEVETPSDLRDI |
| | | GIATIQYKIDNNSRPKVR |
| | | VKLDYVIDDDSKINYFMNHSLLKSRYPDKVLEILKQSTIIEF |
| | | ESSGFNKTIKEMLGMKLA |
| | | GIYNETSNN |
| SEQ | Staphylococcus | MKRNYILGLDIGITSVGYGIIDYETRDVIDAGVRLFKEANVE |
| ID | aureus | NNEGRRSKRGARRLKRRR |
| NO: | | RHRIQRVKKLLFDYNLLTDHSELSGINPYEARVKGLSQKLS |
| 5 | | EEEFSAALLHLAKRRGVHN |
| | | VNEVEEDTGNELSTKEQISRNSKALEEKYVAELQLERLKKD |
| | | GEVRGSINRFKTSDYVKEA |
| | | KQLLKVQKAYHQLDQSFIDTYIDLLETRRTYYEGPGEGSPF |
| | | GWKDIKEWYEMLMGHCTYF |
| | | PEELRSVKYAYNADLYNALNDLNNLVITRDENEKLEYYEK |
| | | FQIIENVFKQKKKPTLKQIA |
| | | KEILVNEEDIKGYRVTSTGKPEFTNLKVYHDIKDITARKEBE |
| | | NAELLDQIAKILTIYQS |
| | | SEDIQEELTNLNSELTQEEIEQISNLKGYTGTHNLSLKAINLI |
| | | LDELWHTNDNQIAIFNR |
| | | LKLVPKKVDLSQQKEIPTTLVDDFILSPVVKRSFIQSIKVINA |
| | | IIKKYGLPNDIIIELAR |
| | | EKNSKDAQKMINEMQKRNRQTNERIEEIIRTTGKENAKYLI |
| | | EKIKLHDMQEGKCLYSLEA |
| | | IPLEDLLNNPFNYEVDHIIPRSVSFDNSFNNKVLVKQEENSK |
| | | KGNRTPFQYLSSSDSKIS |
| | | YETFKKHILNLAKGKGRISKTKKEYLLEERDINRFSVQKDFI |
| | | NRNLVDTRYATRGLMNLL |
| | | RSYFRVNNLDVKVKSINGGFTSFLRRKWKFKKERNKGYKH |
| | | HAEDALIIANADFIFKEWKK |
| | | LDKAKKVMENQMFEEKQAESMPEIETEQEYKEIFITPHQIK |
| | | HIKDFKDYKYSHRVDKKPN |
| | | RELINDTLYSTRKDDKGNTLIVNNLNGLYDKDNDKLKKLI |
| | | NKSPEKLLMYHHDPQTYQKL |
| | | KLIMEQYGDEKNPLYKYYEETGNYLTKYSKKDNGPVIKKI |
| | | KYYGNKLNAHLDITDDYPNS |
| | | RNKVVKLSLKPYRFDVYLDNGVYKFVTVKNLDVIKKENY |
| | | YEVNSKCYEEAKKLKKISNQA |
| | | EFIASFYNNDLIKINGELYRVIGVNNDLLNRIEVNMIDITYRE |
| | | YLENMNDKRPPRIIKTI |
| | | ASKTQSIKKYSTDILGNLYEVKSKKHPQIIKKG |
| SEQ | Streptococcus | MTKPYSIGLDIGTNSVGWAVTTDNYKVPSKKMKVLGNTS |
| ID | thermophilus | KKYIKKNLLGVLLFDSGITAE |
| NO: | (strain ATCC | GRRLKRTARRRYTRRRNRILYLQEIFSTEMATLDDAFFQRL |
| 6 | BAA-491/LMD-9) | DDSFLVPDDKRDSKYPIFG |
| | | NLVEEKAYHDEFPTIYHLRKYLADSTKKADLRLVYLALAH |
| | | MIKYRGHFLIEGEFNSKNND |
| | | IQKNFQDFLDTYNAIFESDLSLENSKQLEEIVKDKISKLEKK |
| | | DRILKLFPGEKNSGIFSE |
| | | FLKLIVGNQADFRKCFNLDEKASLHFSKESYDEDLETLLGY |
| | | IGDDYSDVFLKAKKLYDAI |
| | | LLSGFLTVTDNETEAPLSSAMIKRYNEHKEDLALLKEYIRNI |
| | | SLKTYNEVFKDDTKNGYA |
| | | GYIDGKTNQEDFYVYLKKLLAEFEGADYFLEKIDREDFLRK |
| | | QRTFDNGSIPYQIHLQEMR |
| | | AILDKQAKFYPFLAKNKERIEKILTFRIPYYVGPLARGNSDF |
| | | AWSIRKRNEKITPWNFED |
| | | VIDKESSAEAFINRMTSFDLYLPEEKVLPKHSLLYETFNVYN |
| | | ELTKVRFIAESMRDYQFL |
| | | DSKQKKDIVRLYFKDKRKVTDKDIIEYLHAIYGYDGIELKG |
| | | IEKQFNSSLSTYHDLLNII |
| | | NDKEFLDDSSNEAIIEEIIHTLTIFEDREMIKQRLSKFENIFDK |
| | | SVLKKLSRRHYTGWGK |
| | | LSAKLINGIRDEKSGNTILDYLIDDGISNRNFMQLIHDDALS |
| | | FKKKIQKAQIIGDEDKGN |
| | | IKEVVKSLPGSPAIKKGILQSIKIVDELVKVMGGRKPESIVV |
| | | EMARENQYTNQGKSNSQQ |
| | | RLKRLEKSLKELGSKILKENIPAKLSKIDNNALQNDRLYLY |
| | | YLQNGKDMYTGDDLDIDRL |
| | | SNYDIDHIIPQAFLKDNSIDNKVLVSSASNRGKSDDVPSLEV |
| | | VKKRKTFWYQLLKSKLIS |
| | | QRKFDNLTKAERGGLSPEDKAGFIQRQLVETRQITKHVARL |
| | | LDEKFNNKKDENNRAVRTV |
| | | KIITLKSTLVSQFRKDFELYKVREINDFHEARDAYLNAVVA |
| | | SALLKKYPKLEPEFVYGDY |
| | | PKYNSFRERKSATEKVYFYSNIMNIFKKSISLADGRVIERPLI |
| | | EVNEETGESVWNKESDL |
| | | ATVRRVLSYPQVNVVKKVEEQNHGLDRGKPKGLFNANLS |
| | | SKPKPNSNENLVGAKEYLDPK |
| | | KYGGYAGISNSFTVLVKGTIEKGAKKKITNVLEFQGISILDR |
| | | INYRKDKLNFLLEKGYKD |
| | | IELIIELPKYSLFELSDGSRRMLASILSTNNKRGEIHKGNQIFL |
| | | SQKFVKLLYHAKRISN |
| | | TINENHRKYVENHKKEFEELFYYILEFNENYVGAKKNGKL |
| | | LNSAFQSWQNHSIDELCSSF |
| | | IGPTGSERKGLFELTSRGSAADFEFLGVKIPRYRDYTPSSLL |
| | | KDATLIHQSVTGLYETRI |
| | | DLAKLGEG |
| SEQ | Actinomyces | MWYASLMSAHHLRVGIDVGTHSVGLATLRVDDHGTPIELL |
| ID | naeslundii (strain | SALSHIHDSGVGKEGKKDHD |
| NO: | ATCC 12104/ | TRKKLSGIARRARRLLHHRRTQLQQLDEVLRDLGFPIPTPG |
| 7 | DSM 43013/ | EFLDLNEQTDPYRVWRVRA |
| | JCM 8349/ | RLVEEKLPEELRGPAISMAVRHIARHRGWRNPYSKVESLLS |
| | NCTC 10301/ | PAEESPFMKALRERILATT |
| | Howell 279) | GEVLDDGITPGQAMAQVALTHNISMRGPEGILGKLHQSDN |
| | | ANEIRKICARQGVSPDVCKQ |
| | | LLRAVFKADSPRGSAVSRVAPDPLPGQGSFRRAPKCDPEFQ |
| | | RFRIISIVANLRISETKGE |
| | | NRPLTADERRHVVTFLTEDSQADLTWVDVAEKLGVHRRD |
| | | LRGTAVHTDDGERSAARPPID |
| | | ATDRIMRQTKISSLKTWWEEADSEQRGAMIRYLYEDPTDS |
| | | ECAEIIAELPEEDQAKLDSL |
| | | HLPAGRAAYSRESLTALSDHMLATTDDLHEARKRLFGVDD |
| | | SWAPPAEAINAPVGNPSVDR |
| | | TLKIVGRYLSAVESMWGTPEVIHVEHVRDGFTSERMADER |
| | | DKANRRRYNDNQEAMKKIQR |
| | | DYGKEGYISRGDIVRLDALELQGCACLYCGTTIGYHTCQLD |
| | | HIVPQAGPGSNNRRGNLVA |
| | | VCERCNRSKSNTPFAVWAQKCGIPHVGVKEAIGRVRGWR |
| | | KQTPNTSSEDLTRLKKEVIAR |
| | | LRRTQEDPEIDERSMESVAWMANELHHRIAAAYPETTVMV |
| | | YRGSITAAARKAAGIDSRIN |
| | | LIGEKGRKDRIDRRHHAVDASVVALMEASVAKTLAERSSL |
| | | RGEQRLTGKEQTWKQYTGST |
| | | VGAREHFEMWRGHMLHLTELFNERLAEDKVYVTQNIRLR |
| | | LSDGNAHTVNPSKLVSHRLGD |
| | | GLTVQQIDRACTPALWCALTREKDFDEKNGLPAREDRAIR |
| | | VHGHEIKSSDYIQVFSKRKK |
| | | TDSDRDETPFGAIAVRGGFVEIGPSIHHARIYRVEGKKPVYA |
| | | MLRVFTHDLLSQRHGDLF |
| | | SAVIPPQSISMRCAEPKLRKAITTGNATYLGWVVVGDELEI |
| | | NVDSFTKYAIGRFLEDFPN |
| | | TTRWRICGYDTNSKLTLKPIVLAAEGLENPSSAVNEIVELK |
| | | GWRVAINVLTKVHPTVVRR |
| | | DALGRPRYSSRSNLPTSWTIE |
| SEQ | Neisseria | MAAFKPNSINYILGLDIGIASVGWAMVEIDEEENPIRLIDLG |
| ID | meningitidis | VRVFERAEVPKTGDSLAM |
| NO: | serogroup C | ARRLARSVRRLTRRRAHRLLRTRRLLKREGVLQAANFDEN |
| 8 | (strain 8013) | GLIKSLPNTPWQLRAAALDR |
| | | KLTPLEWSAVLLHLIKHRGYLSQRKNEGETADKELGALLK |
| | | GVAGNAHALQTGDFRTPAEL |
| | | ALNKFEKESGHIRNQRSDYSHTFSRKDLQAELILLFEKQKEF |
| | | GNPHVSGGLKEGIETLLM |
| | | TQRPALSGDAVQKMLGHCTFEPAEPKAAKNTYTAERFIWL |
| | | TKLNNLRILEQGSERPLTDT |
| | | ERATLMDEPYRKSKLTYAQARKLLGLEDTAFFKGLRYGKD |
| | | NAEASTLMEMKAYHAISRAL |
| | | EKEGLKDKKSPLNLSPELQDEIGTAFSLFKTDEDITGRLKDR |
| | | IQPEILEALLKHISFDKF |
| | | VQISLKALRRIVPLMEQGKRYDEACAEIYGDHYGKKNTEE |
| | | KIYLPPIPADEIRNPVVLRA |
| | | LSQARKVINGVVRRYGSPARIHIETAREVGKSFKDRKEIEK |
| | | RQEENRKDREKAAAKFREY |
| | | FPNFVGEPKSKDILKLRLYEQQHGKCLYSGKEINLGRLNEK |
| | | GYVEIDHALPFSRTWDDSF |
| | | NNKVLVLGSENQNKGNQTPYEYFNGKDNSREWQEFKARV |
| | | ETSRFPRSKKQRILLQKFDED |
| | | GFKERNLNDTRYVNRFLCQFVADRMRLTGKGKKRVFASN |
| | | GQITNLLRGFWGLRKVRAEND |
| | | RHHALDAVVVACSTVAMQQKITRFVRYKEMNAFDGKTID |
| | | KETGEVLHQKTHFPQPWEFFA |
| | | QEVMIRVFGKPDGKPEFEEADTLEKLRTLLAEKLSSRPEAV |
| | | HEYVTPLFVSRAPNRKMSG |
| | | QGHMETVKSAKRLDEGVSVLRVPLTQLKLKDLEKMVNRE |
| | | REPKLYEALKARLEAHKDDPA |
| | | KAFAEPFYKYDKAGNRTQQVKAVRVEQVQKTGVWVRNH |
| | | NGIADNATMVRVDVFEKGDKYY |
| | | LVPIYSWQVAKGILPDRAVVQGKDEEDWQLIDDSENFKFSL |
| | | HPNDLVEVITKKARMFGYF |
| | | ASCHRGTGNINIRIHDLDHKIGKNGILEGIGVKTALSFQKYQ |
| | | IDELGKEIRPCRLKKRPP |
| | | VR |
| SEQ | Listeria innocua | MKKPYTIGLDIGTNSVGWAVLTDQYDLVKRKMKIAGDSE |
| ID | serovar 6a (strain | KKQIKKNFWGVRLFDEGQTAA |
| NO: | ATCC BAA-680/ | DRRMARTARRRIERRRNRISYLQGIFAEEMSKTDANFFCRL |
| 9 | CLIP 11262) | SDSFYVDNEKRNSRHPFFA |
| | | TIEEEVEYHKNYPTIYHLREELVNSSEKADLRLVYLALAHII |
| | | KYRGNFLIEGALDTQNTS |
| | | VDGIYKQFIQTYNQVFASGIEDGSLKKLEDNKDVAKILVEK |
| | | VTRKEKLERILKLYPGEKS |
| | | AGMFAQFISLIVGSKGNFQKPFDLIEKSDIECAKDSYEEDLE |
| | | SLLALIGDEYAELFVAAK |
| | | NAYSAVVLSSIITVAETETNAKLSASMIERFDTHEEDLGELK |
| | | AFIKLHLPKHYEEIFSNT |
| | | EKHGYAGYIDGKTKQADFYKYMKMTLENIEGADYFIAKIE |
| | | KENFLRKQRTFDNGAIPHQL |
| | | HLEELEAILHQQAKYYPFLKENYDKIKSLVTFRIPYFVGPLA |
| | | NGQSEFAWLTRKADGEIR |
| | | PWNIEEKVDFGKSAVDFIEKMTNKDTYLPKENVLPKHSLC |
| | | YQKYLVYNELTKVRYINDQG |
| | | KTSYFSGQEKEQIFNDLFKQKRKVKKKDLELFLRNMSHVE |
| | | SPTIEGLEDSENSSYSTYHD |
| | | LLKVGIKQEILDNPVNTEMLENIVKILTVFEDKRMIKEQLQ |
| | | QFSDVLDGVVLKKLERRHY |
| | | TGWGRLSAKLLMGIRDKQSHLTILDYLMNDDGLNRNLMQ |
| | | LINDSNLSEKSIIEKEQVTTA |
| | | DKDIQSIVADLAGSPAIKKGILQSLKIVDELVSVMGYPPQTI |
| | | VVEMARENQTTGKGKNNS |
| | | RPRYKSLEKAIKEFGSQILKEHPTDNQELRNNRLYLYYLQN |
| | | GKDMYTGQDLDIHNLSNYD |
| | | IDHIVPQSFITDNSIDNLVLTSSAGNREKGDDVPPLEIVRKRK |
| | | VFWEKLYQGNLMSKRKF |
| | | DYLTKAERGGLTEADKARFIHRQLVETRQITKNVANILHQR |
| | | FNYEKDDHGNTMKQVRIVT |
| | | LKSALVSQFRKQFQLYKVRDVNDYHHAHDAYLNGVVANT |
| | | LLKVYPQLEPEFVYGDYHQFD |
| | | WFKANKATAKKQFYTNIMLFFAQKDRIIDENGEILWDKKY |
| | | LDTVKKVMSYRQMNIVKKTE |
| | | IQKGEFSKATIKPKGNSSKLIPRKTNWDPMKYGGLDSPNM |
| | | AYAVVIEYAKGKNKLVFEKK |
| | | IIRVTIMERKAFEKDEKAFLEEQGYRQPKVLAKLPKYTLYE |
| | | CEEGRRRMLASANEAQKGN |
| | | QQVLPNHLVTLLHHAANCEVSDGKSLDYIESNREMFAELL |
| | | AHVSEFAKRYTLAEANLNKI |
| | | NQLFEQNKEGDIKAIAQSFVDLMAFNAMGAPASFKFFETTI |
| | | ERKRYNNLKELLNSTIIYQ |
| | | SITGLYESRKRLDD |
| SEQ | Pasteurella | MQTTNLSYILGLDLGIASVGWAVVEINENEDPIGLIDVGVRI |
| ID | multocida (strain | FERAEVPKTGESLALSRR |
| NO: | Pm70) | LARSTRRLIRRRAHRLLLAKRFLKREGILSTIDLEKGLPNQA |
| 10 | | WELRVAGLERRLSAIEWG |
| | | AVLLHLIKHRGYLSKRKNESQTNNKELGALLSGVAQNHQL |
| | | LQSDDYRTPAELALKKFAKE |
| | | EGHIRNQRGAYTHTFNRLDLLAELNLLFAQQHQFGNPHCK |
| | | EHIQQYMTELLMWQKPALSG |
| | | EAILKMLGKCTHEKNEFKAAKHTYSAERFVWLTKLNNLRI |
| | | LEDGAERALNEEERQLLINH |
| | | PYEKSKLTYAQVRKLLGLSEQAIFKHLRYSKENAESATFME |
| | | LKAWHAIRKALENQGLKDT |
| | | WQDLAKKPDLLDEIGTAFSLYKTDEDIQQYLTNKVPNSVIN |
| | | ALLVSLNFDKFIELSLKSL |
| | | RKILPLMEQGKRYDQACREIYGHHYGEANQKTSQLLPAIPA |
| | | QEIRNPVVLRTLSQARKVI |
| | | NAIIRQYGSPARVHIETGRELGKSFKERREIQKQQEDNRTKR |
| | | ESAVQKFKELFSDFSSEP |
| | | KSKDILKFRLYEQQHGKCLYSGKEINIHRLNEKGYVEIDHA |
| | | LPFSRTWDDSFNNKVLVLA |
| | | SENQNKGNQTPYEWLQGKINSERWKNFVALVLGSQCSAA |
| | | KKQRLLTQVIDDNKFIDRNLN |
| | | DTRYIARFLSNYIQENLLLVGKNKKNVFTPNGQITALLRSR |
| | | WGLIKARENNNRHHALDAI |
| | | VVACATPSMQQKITRFIRFKEVHPYKIENRYEMVDQESGEII |
| | | SPHFPEPWAYFRQEVNIR |
| | | VFDNHPDTVLKEMLPDRPQANHQFVQPLFVSRAPTRKMSG |
| | | QGHMETIKSAKRLAEGISVL |
| | | RIPLTQLKPNLLENMVNKEREPALYAGLKARLAEFNQDPA |
| | | KAFATPFYKQGGQQVKAIRV |
| | | EQVQKSGVLVRENNGVADNASIVRTDVFIKNNKFFLVPIYT |
| | | WQVAKGILPNKAIVAHKNE |
| | | DEWEEMDEGAKFKFSLFPNDLVELKTKKEYFFGYYIGLDR |
| | | ATGNISLKEHDGEISKGKDG |
| | | VYRVGVKLALSFEKYQVDELGKNRQICRPQQRQPVR |
| SEQ | Corynebacterium | MKYHVGIDVGTFSVGLAAIEVDDAGMPIKTLSLVSHIHDSG |
| ID | diphtheriae | LDPDEIKSAVTRLASSGIA |
| NO: | (strain ATCC | RRTRRLYRRKRRRLQQLDKFIQRQGWPVIELEDYSDPLYP |
| 11 | 700971/NCTC | WKVRAELAASYIADEKERGE |
| | 13129/Biotype | KLSVALRHIARHRGWRNPYAKVSSLYLPDGPSDAFKAIREE |
| | gravis) | IKRASGQPVPETATVGQMV |
| | | TLCELGTLKLRGEGGVLSARLQQSDYAREIQEICRMQEIGQ |
| | | ELYRKIIDVVFAAESPKGS |
| | | ASSRVGKDPLQPGKNRALKASDAFQRYRIAALIGNLRVRV |
| | | DGEKRILSVEEKNLVFDHLV |
| | | NLTPKKEPEWVTIAEILGIDRGQLIGTATMTDDGERAGARP |
| | | PTHDTNRSIVNSRIAPLVD |
| | | WWKTASALEQHAMVKALSNAEVDDFDSPEGAKVQAFFA |
| | | DLDDDVHAKLDSLHLPVGRAAY |
| | | SEDTLVRLTRRMLSDGVDLYTARLQEFGIEPSWTPPTPRIGE |
| | | PVGNPAVDRVLKTVSRWL |
| | | ESATKTWGAPERVIIEHVREGFVTEKRAREMDGDMRRRAA |
| | | RNAKLFQEMQEKLNVQGKPS |
| | | RADLWRYQSVQRQNCQCAYCGSPITFSNSEMDHIVPRAGQ |
| | | GSTNTRENLVAVCHRCNQSK |
| | | GNTPFAIWAKNTSIEGVSVKEAVERTRHWVTDTGMRSTDF |
| | | KKFTKAVVERFQRATMDEEI |
| | | DARSMESVAWMANELRSRVAQHFASHGTTVRVYRGSLTA |
| | | EARRASGISGKLKFFDGVGKS |
| | | RLDRRHHAIDAAVIAFTSDYVAETLAVRSNLKQSQAHRQE |
| | | APQWREFTGKDAEHRAAWRV |
| | | WCQKMEKLSALLTEDLRDDRVVVMSNVRLRLGNGSAHKE |
| | | TIGKLSKVKLSSQLSVSDIDK |
| | | ASSEALWCALTREPGFDPKEGLPANPERHIRVNGTHVYAG |
| | | DNIGLFPVSAGSIALRGGYA |
| | | ELGSSFHHARVYKITSGKKPAFAMLRVYTIDLLPYRNQDLF |
| | | SVELKPQTMSMRQAEKKLR |
| | | DALATGNAEYLGWLVVDDELVVDTSKIATDQVKAVEAEL |
| | | GTIRRWRVDGFFSPSKLRLRP |
| | | LQMSKEGIKKESAPELSKIIDRPGWLPAVNKLFSDGNVTVV |
| | | RRDSLGRVRLESTAHLPVT |
| | | WKVQ |
| SEQ | Campylobacter | MARILAFDIGISSIGWAFSENDELKDCGVRIFTKVENPKTGE |
| ID | jejuni subsp. | SLALPRRLARSARKRLAR |
| NO: | jejuni serotype | RKARLNHLKHLIANEFKLNYEDYQSFDESLAKAYKGSLISP |
| 12 | O:2 (strain ATCC | YELRFRALNELLSKQDFAR |
| | 700819/NCTC | VILHIAKRRGYDDIKNSDDKEKGAILKAIKQNEEKLANYQS |
| | 11168) | VGEYLYKEYFQKFKENSKE |
| | | FTNVRNKKESYERCIAQSFLKDELKLIFKKQREFGFSFSKKF |
| | | EEEVLSVAFYKRALKDFS |
| | | HLVGNCSFFTDEKRAPKNSPLAFMFVALTRIINLLNNLKNT |
| | | EGILYTKDDLNALLNEVLK |
| | | NGTLTYKQTKKLLGLSDDYEFKGEKGTYFIEFKKYKEFIKA |
| | | LGEHNLSQDDLNEIAKDIT |
| | | LIKDEIKLKKALAKYDLNQNQIDSLSKLEFKDHLNISFKALK |
| | | LVTPLMLEGKKYDEACNE |
| | | LNLKVAINEDKKDFLPAFNETYYKDEVTNPVVLRAIKEYR |
| | | KVLNALLKKYGKVHKINIEL |
| | | AREVGKNHSQRAKIEKEQNENYKAKKDAELECEKLGLKIN |
| | | SKNILKLRLFKEQKEFCAYS |
| | | GEKIKISDLQDEKMLEIDHIYPYSRSFDDSYMNKVLVFTKQ |
| | | NQEKLNQTPFEAFGNDSAK |
| | | WQKIEVLAKNLPTKKQKRILDKNYKDKEQKNFKDRNLND |
| | | TRYIARLVLNYTKDYLDFLPL |
| | | SDDENTKLNDTQKGSKVHVEAKSGMLTSALRHTWGFSAK |
| | | DRNNHLHHAIDAVIIAYANNS |
| | | IVKAFSDFKKEQESNSAELYAKKISELDYKNKRKFFEPFSGF |
| | | RQKVLDKIDEIFVSKPER |
| | | KKPSGALHEETFRKEEEFYQSYGGKEGVLKALELGKIRKV |
| | | NGKIVKNGDMFRVDIFKHKK |
| | | TNKFYAVPIYTMDFALKVLPNKAVARSKKGEIKDWILMDE |
| | | NYEFCFSLYKDSLILIQTKD |
| | | MQEPEFVYYNAFTSSTVSLIVSKHDNKFETLSKNQKILFKN |
| | | ANEKEVIAKSIGIQNLKVF |
| | | EKYIVSALGEVTKAEFRQREDFKK |
| SEQ | Rhodobacteraceae | MRLGLDIGTNSIGWWLCETDRADARVRINGVLAGGVRIFS |
| ID | bacterium | DGRDPKSRASLAVDRRAARA |
| NO: | | MRRRRDRYLRRRATLMKVLANAGLMPSTPEEAKALELLD |
| 13 | | PYELRATGLDQILPLTHLGRA |
| | | LFHINQRRGFKSNRKTDWGDNESGKIKDATARLDLAILAN |
| | | GARTYGEFLHKRRQRAVDPR |
| | | HVPTVRTRLSIANRDGPDGKEEAGYDFYPDRKHLEEEFRKL |
| | | WAAQANFHPELTEDLHDLI |
| | | FEKIFYQRPLKEPKVGLCLFTSEERLPKAHPLTQARVLYETV |
| | | NQLRVIADGRETRRLTLE |
| | | ERDQIIYVLDNKKPTVSLKSMAMKLPALARTLKLRDGERF |
| | | TLETGVRDAIACDPVRSSLS |
| | | HPDRFGPRWSTLDATAQWEVVSRVRKVQSEAEHAALVDW |
| | | LMQAYSIDRNHAEATANAPLP |
| | | EGFGRLGQTATTSILERLKADVVTYAEAVAACGWHHSDQ |
| | | RTGECLDRLPYYGEVLDRHVI |
| | | PGTYDANDDEVTRYGRITNPTVHIGLNQLRRLVNRIIETYG |
| | | KPDQIVLELARELKQSEQQ |
| | | KRDAIKRIRDTTEAAKKRSEKLEELGIEDNGRNRMLLRLWE |
| | | DLNPEDAMRRFCPYTGERI |
| | | SATMIFDGSCDVDHILPYSRTLDDSFANRTLCLKEANREKR |
| | | NQTPWKAWGDAPKWDTIEA |
| | | KLKNLPENKRWRFAPDAMERFEGEKDFLDRALVDTQYLA |
| | | RISRTYMDTLFSEGGHVWVVP |
| | | GRLTEMLRRHWGLNSLLSDKDRGAVKAKNRTDHRHHAID |
| | | AAVVAATDRSLLNRISRAAGQ |
| | | GEAAGQSAELIARDTPPPWEGFRDDLRVQLDKIIVSHRADH |
| | | GRIDREGRKQGRDSTAGQL |
| | | HNDTAYGVVDAMTVVSRTPLLSLKPSDIAVTPKGKNIRDP |
| | | QLQKALEIATRGKEGKAFEA |
| | | ALRQFAEKAGAYQGLRRVRLIETLQESARVEIGTRSEGGPL |
| | | KAYKGDSNHCYELWRLPDG |
| | | KVKPQVVTTYEAHAGIEKRPHPAAKRLLRTFKRDMVALER |
| | | NGETVICYVQKFNQAGILFL |
| | | ASHLESNADARDRDPNDSFTLFRMSPGPMHKAGIRRVSVD |
| | | EIGRLRDGGAETH |
| SEQ | Campylobacter | MKIIGFNLGIANIGWALRENDEIIDCGVRVFDIPENPKNGNS |
| ID | coli | LALERRENKARMKIVKRK |
| NO: | | KARMLATKTFLKKEFNVDLSKLFLIGSTQSIYELRTKALSSL |
| 14 | | ISKEELSAIILHIAKHRG |
| | | YDDSALKNENGTIIEALNKNKEAMLKFKSVGEYFYKNFVQ |
| | | NKEVKKIRNTTEDYSNSVPR |
| | | SLLKQELDLILDKQKELGLIKNADFKAKLFEIIFFKRPLKDFS |
| | | NKIGNCIFFENEKRAAK |
| | | NTISACEFVALGKVVNLLKSIEKDIGIVYEKDSINEIMSIILD |
| | | KTSISYKKIRDILNLPQ |
| | | DINFKGLDYSKNNVENSKLVDLKKLNEFKKALGDGFTNLD |
| | | KDILDSIATDITLTKDTATL |
| | | KEKLKNYNVLNAEQIEKLSELVFNDHINLSLKALKQIIPLM |
| | | YEGKRYDEACELCNFTIAK |
| | | NQEKNEYLPLFEKTRFAKDISSPVVIRAICEFRKLLNDIIRRY |
| | | GSVHKIHLELTRDFGIS |
| | | FNDRKKIIKEIEQNEQSRIKALETIKELKLEETSKNIQIVRLFE |
| | | DQKGICPYSGLKMDLK |
| | | CLDELVIDYIRPYNRSLDDSYSNKVLTFKKLNDLKQGKTPF |
| | | EAFGEDEKLWAEINERIKE |
| | | YNGKKRFKIFDKFFKDKKPFDFTEQTLQDTRWLTKLVASY |
| | | LNEYLSFLPISEDENTALGY |
| | | GEKGSKQHVILSSGMITQMLRNFWYLGFKNHKDYKNNAM |
| | | DAIIVAFTTNSIIFTFNNFKK |
| | | ELDLAKAEFYANKISESDYLLKRKFLPPFSGFKEQALEKVK |
| | | NIFVSHSLKIKNKGTLHEL |
| | | TPLKIKELKNTYGDLDLAVKLGKIRKYNDKYYANAKGSLV |
| | | RTDLFVDKENKFHAVSIYKA |
| | | DFSTKKLPNKTPATTSNGETKEGIEMNENYNFCMSLYKNTP |
| | | IGVKIKGMKESIICYYHGF |
| | | NTSGSKITYKKHDNNYHNLSEDEMVVFRKNDKESIVVGKI |
| | | LEIKKYSISPSGELSLIENE |
| | | KRKWF |
| SEQ | Ignavibacteria | MKNILGLDLGTNSIGWALIDKENNKIIDMGSRIIPMSQDILG |
| ID | bacterium | EFGKGNSISQTAERTNYR |
| NO: | ADurb.Bin266 | SIRRLRERYLLRRERLHRVLNILEFLPKHYSDQIDFETRLGK |
| 15 | | FKEDTEPKIAYKSTIDET |
| | | NSKSRFDFIFKKSFAEMLEDFHQYQPELFANDNKIPYDWTI |
| | | YFLRKKALTKKIEKEELAW |
| | | ILLNFNQKRGYYQLREELEEDTNKKEYVVSLKVIKIVKGEE |
| | | DKKNKNRNWYSISLENGWV |
| | | YNATFSTEPQWLMTEKEFLVTEELDENGQVKIVKDKKSDK |
| | | EGKEKRRIIPLPSFDEINLM |
| | | SKSEPDRIYKKIKAKTETAISNSGKTVGEYIYENLLQNPSQK |
| | | IRGKLIRTIERKFYKEEL |
| | | KQILQKQKEFHPELQNDDLYNDCVRELYKNNEGHQFLLSK |
| | | RDFIHLLLDDIIFYQRPLKS |
| | | QKSLISNCTFEFKKYNVGNEEKIKYLKAIPKSHPLYQEFRF |
| | | WQWIYNLRVYRKDDDQDVT |
| | | NDYLNDPEKYADLFEFLSNRKEIDQKALLKYFKLKESTHR |
| | | WNFVEDKKYPCFETRTLIST |
| | | RLEKVKDLPPNFLTDQTELQLWHIIYSVTDKIEFEKALSTFA |
| | | KRNKLDVTTFVENFKKFP |
| | | PFKSEYGSYSGKALKKLLPLMRSGRYWKWDDIDEKTKTRI |
| | | DKIITGEFDEDIKNKVREKS |
| | | INLTTENHFQGLQVWLASYIVYDRHAEAATINKWDTIEHLE |
| | | NYIKEFKQHSLRNPIVEQV |
| | | TLEALRVIKDIWKQFGKSAENFFDEIHIELGREMKNTADER |
| | | KRLTSQINDNENTNVRIKA |
| | | LLAELKNDSNIENVRPFSPIQQELLKIYEDGVLNSEIEIPDDIS |
| | | KISKTAQPSSSELQRY |
| | | KLWLEQKYRSPYTGQVIPLAKLFTTDYEIEHIIPQSRYFDDS |
| | | FNNKVICEAAVNKLKDNQ |
| | | TGLEFIKNHHGEIVQTVFDNKVKIFEENDYRDFVKTHYIKN |
| | | RSKRNKLLMEEIPDKMIER |
| | | QINDTRYITKFISALLSNIVRAENNDEGLNSKNLIQVNGKITS |
| | | LLRQDWGINDIWNDLIL |
| | | PRFLRMNQITNSDAFTRYNDKYQKYLPTVPLELSKNYQSK |
| | | RIDHRHHALDALIIACATRD |
| | | HVNLLNNKYAKSKERYDLNRKLRLFEKVVYTHPKTGEKIE |
| | | REIPKNFIKPWDTFTVDTKN |
| | | FLDTIVVSFKQNLRIINKATNQYQKWVKLNGRNVKKEVKQ |
| | | SGINWAIRKPLHKETVAGKV |
| | | ELKRIKVPKGKILTATRKNLDTSFDIKTIESITDTGIQKILKN |
| | | YLSAKGNDPTIAFSPEG |
| | | IEEMNKNITRYNNGKPHRPIYKARIFELGSKFILGLTGNKKA |
| | | KYVEAAKGTNLFYAIYVD |
| | | ENNKRSFETIPLNIVIERQKQGLSSVPENDDKGNKLLFYLSP |
| | | NDLVYVPDEDEIINESYL |
| | | DVSNLSNEQKKRLYNVNDFSSTCYFTPNRIAKAIAPKEVDL |
| | | NYDNNKKKLFGSYDTKTAS |
| | | VNGIQIKDICIKLKADRLGNISKANR |
| SEQ | Fructobacillus sp. | MGYNIGLDIGTGSVGWAALTDEGKLARAKGKNLIGVRLFD |
| ID | EFB-N1 | SAQSAAQRRSYRTTRRRLSR |
| NO: | | RKWRLRLLENIFSDEMGMIDENFFARLKYSYVHPKDEVNN |
| 16 | | AHYYGGYLFPTQQETHDFHE |
| | | KFQTIYHLRLKLMIEDCKFDLREIYLAMHHIVKYRGHFLNS |
| | | QSKMTIGDSYNPRDFQQAI |
| | | QNYAEAKGLIWSLNDAQEMTDVLVGQAGFGLSKKAKAER |
| | | LLSAFSFDTKEDKKAIQAILA |
| | | GIVGNTTDFTKIFNRERSGDELKKWKLKLDSEAFDEQSQAI |
| | | VDELDDDEMELFNAIRQAF |
| | | DGFTLMDLLGDQTSISAAMVKRYQQHHDDLKMVKEIAKK |
| | | QGLSHQDFSKIYTAFLKDDTD |
| | | KGMKALLDKADLADDVLVEIQQRIESHDFLPKQRTKANSV |
| | | IPYQLHLAELEKIIENQGKY |
| | | YPFLLDTFTNKAGETINKLVELVKFRVPYYVGPMVTAADV |
| | | EKAGGDATNHWVKRNEGYEK |
| | | SPVTPWNFDQVFNRDQAAQDFIDRLTGTDTYLIGEPTLLKN |
| | | SLKYQLFTVLNELNNVKIN |
| | | GHKIDEKTKHVLIQDLFKSKKTVSEKAIKDYYLSQGMGEIQ |
| | | IVGLADKTKFNSNLSSYID |
| | | LSKTFDAEFMENPANQELLENIIQIQTVFEDVKIAERELQKL |
| | | ALPDEQVQQLAKTHYTGW |
| | | GNLSDKLLSTPIIQEGSQKVSILNKLQTTSKNFMSIITDNKFG |
| | | VQQWIQEQNTAETADSI |
| | | QDRIDELTTAPANKRGIKQAFNVLFDIQKAMGEEPNRVYLE |
| | | FAKETQNSVRTNSRYNRLK |
| | | DLYKSKTLSDDVKALKEELESQKSSLQSERIGDRLYLYFLQ |
| | | QGKDMYTGQPINIDKLSTD |
| | | YDIDHIIPQAYTKDDSIDNRVLVSRPENARKSDSATYTTEVQ |
| | | QSAGGLWKSLKNAGFISQ |
| | | KKYDRLTKGGDYSKGQKTGFIARQLVETRQIIKNVASHES |
| | | EFSQTKAVAIRSEITADMR |
| | | RLVAIKKHREINSFHHAFDALLITAAGQYMQARYPDRDGA |
| | | NVYNEFDYYTNTYLKELRQS |
| | | SSSSQVRRLKPFGFVVGTMAKGNENWSEDDTQYLRHVMN |
| | | FKNILTTRRNDKDNGALNKET |
| | | IYAVDPKAKLIGTNKKRQDVSLYGGYIYPYSAYMTLVRAN |
| | | GKNLLVKVTISAAEKIKSGQ |
| | | IELSEYVQQRPEVKKFEKILINKLAIGQLVNNDGNLIYLTSY |
| | | EFYHNAKQLWLPTEEADL |
| | | ISQLNKDSSDEDLIKGFDILTSPAILKRFPFYELDLKKLVNIR |
| | | DKFIAVENKFDILMVIL |
| | | KALQLDAAQQKPVKMIDKKSADWKDYRQRGGIKLSDTSEI |
| | | IYQSTTGIFEKRVKISNLL |
| SEQ | Pedobacter | MTKHILGLDLGTNSIGWAIIQVDNNNNVPIQIIAMGSRIIPLD |
| ID | glucosidilyticus | SNDRDQFQKGQAISKNK |
| NO: | | DRTTARTQRKGYDRKQLKKSDDFKYSLKKILEKLDIFPTEE |
| 17 | | LMKLPTLDLWKLRSDAVSN |
| | | IEDITPKQLGRILYMLNQKRGYKSARSEANADKKDTDYVA |
| | | EVKGRYTQLKDKGQTLGQYF |
| | | YKELSDANQNNTYYRVKEKVYPREAYIEEFDAIINVQKSK |
| | | HSFLTDEVIHSLRNEIIYYQ |
| | | RKLKSQKGLVSICEFEGFETTYFDKKTQQDKTIFTGPKVAP |
| | | RTSPLFQFCKIWEVVNNIS |
| | | LKTKNPEGSKYKWSDRIPTIEEKQTIANYLQENENLSFJELL |
| | | KILQLKKEQVYANKQILK |
| | | GIQGNTTFSAIHKIIGNSEHLKFDIETIPSKHFAVLVDKKTGE |
| | | ILDERDSLELNSALEQE |
| | | PFYQLWHTIYSIKDLDECKKALIKRFNFEEEIAEKLSKIDFN |
| | | KQAFGNKSNKAMRKMLPY |
| | | LMLGYNQSEAESFAGYNRRLTKEEKSKNVSDEPLQLLAKN |
| | | SLRQPVVEKILNQMINVVNA |
| | | IIEKYGKPEEIRVELARELKQSKDEREDADKQNGFNKKLNE |
| | | LVATKLTELGLPTTKHYIQ |
| | | KYKFIFPAKDKNWKEAQVANQCIYCGDTFNLTEALSGDNF |
| | | DVDHIVPKALLFDDSQANKV |
| | | LVHRSCNSTKTNNTAYDYITKKGSQALNDYVARVDDWFK |
| | | RGIISYGKMQRLKVSFEEYQE |
| | | RKKIGKETEADKRIWENFIDRQLRETAYIAKKAKEILEKVC |
| | | HNVTSTEGNVTAKLRQLWG |
| | | WDNVLMNLQLPKYKELEKKTKQTFTQLKEWTSDHGNRK |
| | | HQKEEIINWTKRDDHRHHAIDA |
| | | LVIACTQQGFIQRINTLSSSDVKDEMKKELEEDKTVYNERL |
| | | TLLENYLLEKKPFSTEEIE |
| | | KEADKILVSFKAGKKVATLSKYKATGINEIKGVLVPRGPLH |
| | | EQSVYGKIKVIEKDKPLKY |
| | | LFENSDKIVNPLIKHLVKTRLLENENNAQAALVTLKNKPIL |
| | | LNNKQTEILEKASCYNEAT |
| | | VLKYKLQSLKASQIDDIVDEKIKFLIKERLSKFGNKEKEAFK |
| | | DILWFNEKKQIPITSIRL |
| | | FARPDANNLQVIKKHEKGKNIGFVLSGNNHHIAIYEDKNN |
| | | KLIQHICDFWHAVERKRNNI |
| | | PVLIEDTSTIWNHLINEDFSESFLNKLPNDSLKLKFSLQQNE |
| | | MFILGLPKEQSEEAIKSN |
| | | NKSLLSKHLYLVWSITDGDYFFRHHLETKNTELKKIDGSKE |
| | | SKRYLRLSTKSLVDLNPIK |
| | | VRLNHLGEITKIGE |
| SEQ | Geobacillus | MKYKIGLDIGITSIGWAVINLDIPRIEDLGVRIFDRAENPKTG |
| ID | thermo- | ESLALPRRLARSARRRL |
| NO: | denitrificans | RRRKHRLERIRRLFVREGILTKEELNKLFEKKHEIDVWQLR |
| 18 | | VEALDRKLNNDELARILLH |
| | | LAKRRGFRSNRKSERTNKENSTMLKHIEENQSILSSYRTVA |
| | | EMVVKDPKFSLHKRNKEDN |
| | | YTNTVARDDLEREIKLIFAKQREYGNIVCTEAFEHEYISTWA |
| | | SQRPFASKDDIEKKVGFC |
| | | TFEPKEKRAPKATYTFQSFTVWEHINKLRLVSPGGIRALTD |
| | | DERRLIYKQAFHKNKITFH |
| | | DVRTLLNLPDDTRFKGLLYDRNTTLKENEKVRFLELGAYH |
| | | KIRKAIDSVYGKGAAKSFRP |
| | | IDFDTEGYALTMEKDDTDIRSYLRNEYEQNGKRMENLADK |
| | | VYDEELIEELLNLSFSKFGH |
| | | LSLKALRNILPYMEQGEVYSTACERAGYTFTGPKKKQKTV |
| | | LLPNIPPIANPVVMRALTQA |
| | | RKVVNAIIKKYGSPVSIHIELARELSQSFDERRKMQKEQEG |
| | | NRKKNETAIRQLVEYGLTL |
| | | NPTGLDIVKFKLWSEQNGKCAYSLQPIEIERLLEPGYTEVD |
| | | HVIPYSRSLDDSYTNKVLV |
| | | LTKENREKGNRTPAEYLGLGSERWQQFETFVLTNKQFSKK |
| | | KRDRLLRLHYDENEENEFKN |
| | | RNLNDTRYISRFLANFIREHLKFADSDDKQKVYTVNGRITA |
| | | HLRSRWNFNKNREESNLHH |
| | | AVDAAIVACTTPSDIARVTAFYQRREQNKELSKKTDPQFPQ |
| | | PWPHFADELQARLSKNPKE |
| | | SIKALNLGNYDNEKLESLQPVFVSRMPKRSITGAAHQETLR |
| | | RYIGIDERSGKIQTVVKKK |
| | | LSEIQLDKTGHFPMYGKESDPRTYEAIRQRLLEHNNDPKKA |
| | | FQEPLYKPKKNGELGPIIR |
| | | TIKIIDTTNQVIPLNDGKTVAYNSNIVRVDVFEKDGKYYCV |
| | | PIYTIDMMKGILPNKAIEP |
| | | NKPYSEWKEMTEDYTFRFSLYPNDLIRIEFPREKTIKTAVGE |
| | | EIKIKDLFAYYQTIDSSN |
| | | GGLSLVSHDNNFSLRSIGSRTLKRFEKYQVDVLGNIYKVRG |
| | | EKRVGVASSSHSKAGETIR |
| | | PL |

Further, in some embodiments, fragments of Cas9 or other programmable nuclease that retain DNA cleaving function can be used to generate the fusion proteins. For example, a Cas9 or other programmable nuclease polypeptide fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to a wild type Cas9. In some embodiments, the Cas9 fragment may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9.

The Cas9 enzymes or other programmable nuclease disclosed herein also comprises at least one nuclear localization signal (NLS), which is an amino acid sequence that attaches to a protein for import into the cell nucleus by nuclear transport. Generally, the NLS comprises one or more short sequences of positively charged lysines or arginines exposed on the protein surface. These types of classical NLSs can be further classified as either monopartite or bipartite. The major structural difference between the two is that the two basic amino acid clusters in bipartite NLSs are separated by a relatively short spacer sequence (hence bipartite-2 parts), while monopartite NLSs are not. In some embodiments, the NLS comprises sequence PKKKRKV (SEQ ID NO: 19) of the SV40 Large T-antigen (a monopartite NLS). In other embodiments, the NLS of nucleoplasmin comprises sequence KR[PAATKKAGQA]KKKK (SEQ ID NO: 20). There are also many other types of non-classical NLSs. Different types of NLSs disclosed herein are not meant to be limiting and a person of ordinary skill in the art is able to select a NLS to attach to a Cas9 protein. In some embodiments, the Cas9 protein comprises an N-terminal NLS. In other embodiments, the Cas9 protein comprises a C-terminal NLS. In yet other embodiments, the Cas9 protein comprises both N-terminal and C-terminal NLSs.

In some embodiments, the other CRISPR-related programmable endonucleases often includes CRISPR-associated (Cas) polypeptides or Cas nucleases including Class 1 Cas polypeptides, Class 2 Cas polypeptides, type I Cas polypeptides, type II Cas polypeptides, type III Cas polypeptides, type IV Cas polypeptides, type V Cas polypeptides, and type VI CRISPR-associated (Cas) polypeptides, CRISPR-associated RNA binding proteins, or a functional fragment thereof. Further, Cas polypeptides suitable for use with the present disclosure often include Cpf1 (or Cas12a), c2c1, C2c2 (or Cas13a), Cas13, Cas13a, Cas13b, c2c3, Cas1, Cas1B, Cas2, Cas3, Cas4, Cas5, Cas5e (CasD), Cash, Cas6e, Cas6f, Cas7, Cas8a, Cas8a1 , Cas8a2, Cas8b, Cas8c, Csn1, Csx12, Cas10, CaslOd, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966; any derivative thereof; any variant thereof; and any fragment thereof.

Additionally, other site-specific endonucleases that are suitable for the fusion protein composition disclosed herein often comprise zinc finger nucleases (ZFN); transcription activator-like effector nucleases (TALEN); meganucleases; RNA-binding proteins (RBP); recombinases; flippases; transposases; Argonaute (Ago) proteins (e.g., prokaryotic Argonaute (pAgo), archaeal Argonaute (aAgo), and eukaryotic Argonaute (eAgo)); or any functional fragment thereof.

hExo1 Protein

A programmable nuclease is often tethered to an exonuclease domain so as to effect the results disclosed herein. A number of exonuclease/programmable exonuclease combinations are consistent with the disclosure herein. With respect to the exonuclease, certain exemplary exonucleases suitable for use as part of the fusion protein in present application include MRE11, EXO1, EXOIII, EXOVII, EXOT, DNA2, CtIP, TREX1, TREX2, Apollo, RecE, RecJ, T5, Lexo, RecBCD, and Mungbean. Additional suitable exonucleases are also contemplated. In certain embodiments, human Exo1 (hExo1) is used herein as a part of the fusion protein. Full length hExo1 can be divided into roughly two regions: the N-terminal nuclease region (1-392) (SEQ ID NO: 1) MGIQGLLQFI KEASEPIHVR KYKGQVVAVD TYCWLHKGAI ACAEKLAKGE PTDRYVGFCM KFVNMLLSHG IKPILVFDGC TLPSKKEVER SRRERRQANL LKGKQLLREG KVSEARECFT RSINITHAMA HKVIKAARSQ GVDCLVAPYE ADAQLAYLNK AGIVQAIITE DSDLLAFGCK KVILKMDQFG NGLEIDQARL GMCRQLGDVF TEEKFRYMCI LSGCDYLSSL RGIGLAKACK VLRLANNPDI VKVIKKIGHY LKMNITVPED YINGFIRANN TFLYQLVFDP IKRKLIPLNA YEDDVDPETL SYAGQYVDDS IALQIALGNK DINTFEQIDD YNPDTAMPAH SRSHSWDDKT CQKSANVSSI WHRNYSPRPE SGTVSDAPQL KE), and the C-terminal MLH2/MSH1 interaction region (393-846). In some embodiments, the N-terminal nuclease region of hExo1(SEQ ID NO: 1) is used to covalently link to a Cas9 with at least one NLS via a peptidyl linker. In other embodiments, a fragment of SEQ ID NO: 1 or other exonuclease domain that retains the nuclease function is used herein. For example, the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to SEQ ID NO: 1. In some embodiments, the fragment may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to SEQ ID NO: 1 or other untruncated or unmutated domain. The N-terminal nuclease region of the hExo1is exemplary, and additionally suitable Exo1 or other exonuclease sequences can be utilized for the purpose disclosed herein by a person of ordinary skill in the art.

An exonuclease such as a hExo1 peptide is connected to a programmable endonuclease such as a Cas9 peptide and at least one NLS in some cases using a linker. In some embodiments, the linker is a linker peptide. The linker peptides not only serves to connect the protein moieties, but in some cases also provides many other functions, such as maintaining cooperative inter-domain interactions or preserving biological activity (Gokhale R S, Khosla C. Role of linkers in communication between protein modules. Curr Opin Chem Biol. 2000;4: 22-27; Ikebe M, Kambara T, Stafford W F, Sata M, Katayama E, Ikebe R. A hinge at the central helix of the regulatory light chain of myosin is critical for phosphorylation-dependent regulation of smooth muscle myosin motor activity. J Biol Chem. 1998; 273: 17702-17707; and Chen X Y, Zaro J, and Shen W C. Fusion protein linkers: property, design and functionality. Adv Drug Deliv Rev 2014; 65, 1357-1369 are incorporated herein). The linker peptides can be grouped into small, medium, and large linkers with average length of less than or up to 4.5±0.7, 9.1±2.4, and 21.0±7.6 residues or greater, respectively, although examples anywhere within the set defined by these three ranges are also contemplated. In some embodiments, the linker peptide comprises 5 to 200 amino acids. In other embodiments, the linker peptide comprises 5 to 25 amino acids. In certain embodiments, the linker peptide is selected from the group consisting of FL2X (encoded by SEQ ID NO: 122 (ggtctccttaaacctgtcttgt)), SLA2X (encoded by SEQ ID NO: 123 (GGAGGTGGAGGCTCTGGTGGAGGCGGATCA)), APSX (encoded by SEQ ID NO: 124 (GCAGAGGCTGCAGCCGCTAAGGCC)), FL1X (encoded by SEQ ID NO: 125 (GCAGAGGCTGCAGCCGCTAAGGAGGCAGCTGCCGCTAAGGCC)), SLA1X, (encoded by SEQ ID NO: 126 (GCACCTGCTCCAGCGCCCGCACCAGCTCCC)) and any combinations thereof. In some embodiments, the linker peptide is SLA2X. Again, these disclosed linker peptides are not meant to be limiting. A person of ordinary skill in the art would be able to select an appropriate linker peptide.

The fusion protein disclosed herein can be fused together directly post-translationally or translated from a polynucleotide (fusion nucleotide) that encodes the disclosed fusion protein in a common open reading frame. In some embodiments, a first nucleic acid sequence encoding hExo1or the N-terminal nuclease region thereof is ligated to one end of a second nucleic acid sequence encoding a selected linker peptide. Further, the other end of the second nucleic acid sequence is ligated with a third nucleic acid sequence encoding Cas9 enzyme with at least one NLS. Generally, stop codons of the first, second, and third nucleic acid sequences are removed. In some embodiments, the first, second and third nucleic acid sequences are codon optimized or engineered for more efficient transfection or expression in a target cell. Similarly, in some instances, intronic sequences are removed.

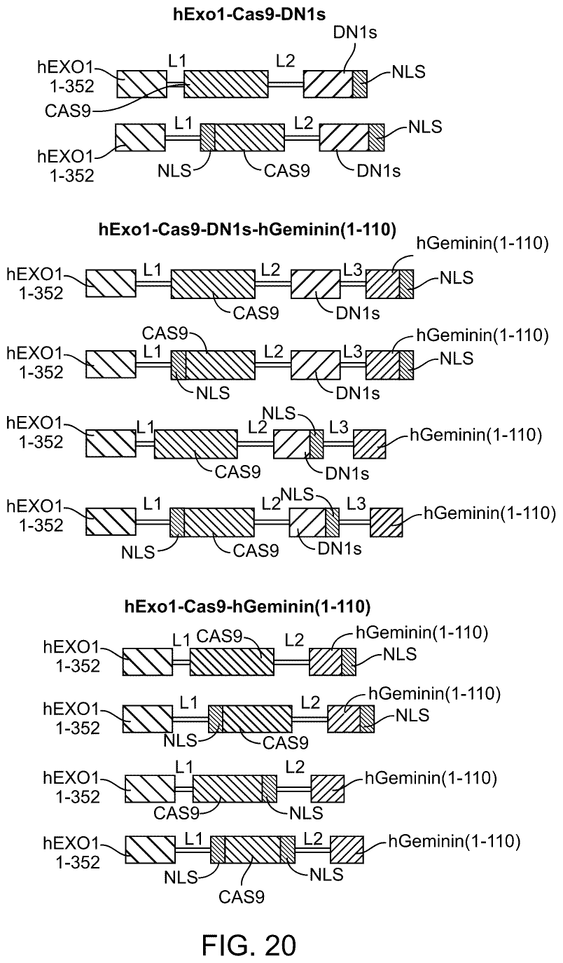

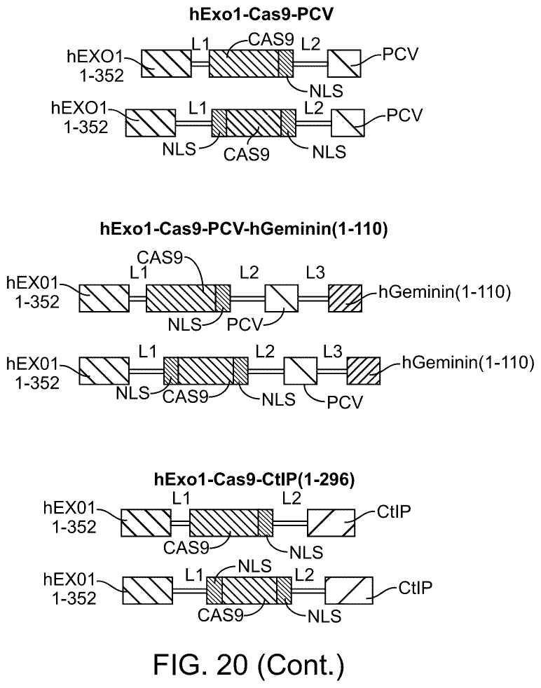

FIG. 20 illustrates exemplary fusion proteins with various arrangements of nucleases, Cas9, and other functional domains connected by linkers (L1, L2, and L3). Additional non-limiting examples of the fusion proteins include: hExo1-Cas9-DN1s (or reverse orientation DN1s-Cas9-HR); hExo1-Cas9-DN1s-Geminin(1-110) (or DN1s-Cas9-HR-Geminin); hExo1-Cas9-Geminin(1-110) (or Cas9-hExo1-Geminin); hExo1-Cas9-PCV (or PCV-Cas9-hExo1); hExo1-Cas9-PCV-Geminin(1-110) (or PCV-Cas9-hExo1-Geminin); and hExo1-Cas9-CtIP(1-296) (or CtIP-Cas9-hExo1).

In some embodiments, hExo1-Cas9-DN1s (or reverse orientation DN1s-Cas9-HR) can be a fusion of hExo1(1-352) via linker 1(FL1X, AP5X or other) to Cas9 possessing or lacking an N-terminal FLAG+NLS (noted as NLS in FIG. 20) subsequently fused via linker 2 (either TGS or other) to a fragment of human p53 (1231-1644) with an NLS sequence added at the C-Terminus. In some embodiments, Cas9-HR and Cas9-DN1s can be acting at different steps in the homologous recombination pathway. In some embodiments, the HR-Cas9-DN1s can have increased error free editing efficiency relative to either Cas9-HR or Cas9-DN1s. In some embodiments, cellular toxicity can be greatly reduced relative to increase seen with Cas9-DN1s when compared to Cas9 alone.

In some instances, hExo1-Cas9-DN1s-Geminin(1-110) (or DN1s-Cas9-HR-Geminin) can be fusion of hExo1(1-352) via linker 1(FL1X, AP5X or other) to Cas9 possessing or lacking an N-terminal FLAG+NLS subsequently fused via linker 2 (either TGS or other) to a fragment of human p53 (1231-1644). DN1s can either have an NLS added to its C-Terminus, which can then be fused to Geminin(1-110) via L3 (any sequence), or fused to Geminin with an NLS sequence at its C-Terminus, which can be fused to DN1s via L3. In some embodiments, the cellular toxicity of the hExo1-Cas9-DN1s-Geminin(1-110) (or DN1s-Cas9-HR-Geminin) can be reduced compared to Cas9. In some embodiments, error free editing efficiency of hExo1-Cas9-DN1s-Geminin(1-110) (or DN1s-Cas9-HR-Geminin) can be increased compared to Cas9. In some embodiments, the error free editing efficiency of hExo1-Cas9-DN1s-Geminin(1-110) (or DN1s-Cas9-HR-Geminin) can be increased compared to Cas9 due to post-translational regulation via geminin of hExo1-Cas9-DN1s-Geminin restricting nuclease activity to S/G2 phase, when endogenous HR is highest in the cell.

In some embodiments, hExo1-Cas9-Geminin(1-110) (or Cas9-hExo1-Geminin) can be a fusion of hExo1(1-352) via linker 1(FL1X, AP5X or other) to Cas9 possessing or lacking an N-terminal FLAG+NLS, possessing or lacking a C-terminal NLS sequence subsequently fused via linker 2 (either TGS or other) to a fragment of Geminin (1-110) either possessing or lacking a C-terminal NLS sequence. In some embodiments, hExo1-Cas9-Geminin(1-110) (or Cas9-hExo1-Geminin) comprises reduced cellular toxicity and increased error free editing efficiency compared to Cas9.

In some embodiments, hExo1-Cas9-PCV (or PCV-Cas9-hExo1 can be a fusion of hExo1(1-352) via linker 1 (FL1X, AP5X or other) to Cas9 possessing or lacking an N-terminal FLAG+NLS, possessing or lacking a C-terminal NLS sequence subsequently fused via linker 2 (either TGS or other) to PCV. In some embodiments, PCV can bind to a specific ssDNA sequence thereby tethering the repair template to the Cas9 complex. In some embodiments, hExo1-Cas9-PCV comprises increased error free editing efficiency compared to Cas9. In some embodiments, hExo1-Cas9-PCV comprises reduced cellular toxicity compared to Cas9.

In some embodiments, hExo1-Cas9-PCV-Geminin(1-110) (or PCV-Cas9-hExo1-Geminin) can be a fusion of hExo1(1-352) via linker 1(FL1X, APSX or other) to Cas9 possessing or lacking an N-terminal FLAG+NLS, possessing or lacking a C-terminal NLS sequence subsequently fused via linker 2 (either TGS or other) to PCV, which can then be fused to a fragment of Geminin (1-110). In some embodiments, hExo1-Cas9-PCV-Geminin(1-110) (or PCV-Cas9-hExo1-Geminin) comprises higher error free editing efficiency compared to Cas9. In some embodiments, hExo1-Cas9-PCV-Geminin(1-110) (or PCV-Cas9-hExo1-Geminin) comprises higher error free editing efficiency compared to Cas9 due to restriction of nuclease activity to S/G2 phase.

In some embodiments, hExo1-Cas9-CtIP(1-296) (or CtIP-Cas9-hExo1) can be a fusion of hExo1(1-352) via linker 1(FL1X, APSX or other) to Cas9 possessing or lacking an N-terminal FLAG+NLS, possessing or lacking a C-terminal NLS sequence subsequently fused via linker 2 (either TGS or other) to CtIP. In some embodiments, CtIP can improve error free editing efficiency compared to Cas9 without CtIP. In some embodiments, CtIP can improve error free editing efficiency compared to Cas9 via binding downstream of blocked DSBs (double-strand breaks) and resecting back towards the break using 3′-5′ exonuclease activity.

Escheria Coli (E. Coli) version of Exo I

In certain embodiments, the Escheria Coli (E. Coli) version of Exo I (E. Coli Exo1) is used herein as a part of the fusion protein. E. coli Exo1 possesses 3′to 5′ exonuclease activity as opposed to the 5′ to 3′ exonuclease activity of hExo1. The E. Coli Exo1 Cas9 fusion can generate much longer deletions than traditional Cas9.

### Nucleic Acid Sequence

Some nucleotide constructs consistent with the disclosure comprise nucleic acid encoding an exonuclease such as hExo1. Further, some nucleotide constructs consistent with the disclosure comprise nucleic acid encoding a programmable endonuclease such as a Cas9 or other CRISPR-related programmable endonucleases. In some embodiments, the nucleic acid sequence encoding hExo1 or the N-terminal nuclease region thereof is non-naturally occurring, but the hExo1 or the N-terminal nuclease region thereof encoded by it has an amino acid sequence that is naturally occurring. In some instances, the nucleic acid sequence is different from a naturally occurring hExo1 or the N-terminal nuclease region thereof nucleic acid sequence but encodes a polypeptide identical to hExo1 or the N-terminal nuclease region thereof owning to codon degeneracy. Similarly, the third nucleic acid sequence encoding Cas9 enzyme with at least one NLS is non-naturally occurring, but the Cas9 protein encoded by it has an amino acid sequence that is naturally occurring. In some instances, the nucleic acid sequence is different from a naturally occurring Cas9 nucleic acid sequence but encodes a polypeptide identical to Cas9 owning to codon degeneracy.

### Ribonucleoprotein (RNP)

A ribonucleoprotein (RNP) typically comprises at least two parts: one part comprises a programmable endonuclease such as a Cas9 or other CRISPR-related programmable endonucleases; and the other part comprises a gRNA or other specificity-conveying nucleic acid. Often, a wild type Cas9 enzyme or other Cas or non-Cas programmable endonuclease can be one part of the CRISPR-Cas9 system. The modified Cas9 protein coupled to a fragment of hExo1via a linker peptide can also be one part of the CRISPR-Cas9 system. Further, the modified Cas9 protein and a gRNA can form a ribonucleoprotein (RNP).

gRNA

A ribonucleic acid that comprises a sequence for guiding the ribonucleic acid to a target site on a gene and another sequence for binding to an endonuclease such as Cas9 enzyme is used herein. Often, the ribonucleic acid is a gRNA. In some embodiments, the gRNA is a synthetic gRNA (sgRNA). The gRNA directs the fusion protein complex to a targeted nucleotide sequence of the DNA molecule. The gRNA is a short synthetic RNA composed of a scaffold sequence necessary for Cas-binding and a user-defined about 20 nucleotide spacer that defines the genomic target to be modified. In certain embodiments, a spacer of a gRNA can be designed to recognize the exon 1 of HBB gene. Thus, one can change the genomic target of the Cas protein by simply changing the target sequence present in the gRNA.

There are several ways to deliver gRNA into cells. One is to deliver gRNA into the cells as plasmid DNA. In some embodiments, the nucleic acids encoding the fusion proteins can be cloned into one plasmid or other suitable vectors with a nucleic acid sequence encoding a designed gRNA targeting a gene of interest.

A list of representative gRNA constituents is provided below.

TABLE 2
A list of gRNA sequences.

| Seq ID No. | Gene Name | Guide Name | Guide Sequence 5′-3′ |
|---|---|---|---|
| SEQ ID NO: 21 | HBB | HBB-1 | GTAACGGCAGACTTCTCCTC |
| SEQ ID NO: 22 | HBB | HBB-2 | GTCTGCCGTTACTGCCCTGT |
| SEQ ID NO: 23 | HBB | HBB-3 | GAGGTGAACGTGGATGAAGT |
| SEQ ID NO: 24 | HBG1 | HBG1-1 | TATCTGTCTGAAACGGTCCC |
| SEQ ID NO: 25 | HBG1 | HBG1-2 | GCTAAACTCCACCCATGGGT |
| SEQ ID NO: 26 | HBG1 | HBG1-3 | CAAGGCTATTGGTCAAGGCA |
| SEQ ID NO: 27 | BCL11A | BCL11A-1 | AAATAAGAATGTCCCCCAAT |
| SEQ ID NO: 28 | BCL11A | BCL11A-2 | CACAAACGGAAACAATGCAA |
| SEQ ID NO: 29 | BCL11A | BCL11A-3 | AATATCATTTCTGTTCAAAA |
| SEQ ID NO: 30 | CCR5 | CCR5-1 | TAATAATTGATGTCATAGAT |
| SEQ ID NO: 31 | CCR5 | CCR5-2 | TGACATCAATTATTATACAT |
| SEQ ID NO: 32 | CCR5 | CCR5-3 | CTTTTTATTTATGCACAGGG |
| SEQ ID NO: 33 | CXCR4 | CXCR4-1 | ATCCCCTCCATGGTAACCGC |
| SEQ ID NO: 34 | CXCR4 | CXCR4-1 | ACTTACACTGATCCCCTCCA |
| SEQ ID NO: 35 | PPP1R12C | PPP1R12C-1 | GGAGAGGATGGCCCGGCGGC |
| SEQ ID NO: 36 | PPP1R12C | PPP1R12C-2 | ATGGCCCGGCGGCTGGCCCG |
| SEQ ID NO: 37 | PPP1R12C | PPP1R12C-3 | GGATGGCCCGGCGGCTGGCC |
| SEQ ID NO: 38 | HPRT | HPRT-1 | TAGGTATGCAAAATAAATCA |
| SEQ ID NO: 39 | HPRT | HPRT-2 | CATACCTAATCATTATGCTG |
| SEQ ID NO: 40 | HPRT | HPRT-3 | TAAATTCTTTGCTGACCTGC |
| SEQ ID NO: 41 | HPRT | HPRT-4 | TGTAGCCCTCTGTGTGCTCA |
| SEQ ID NO: 42 | HPRT | HPRT-5 | AACTAGAATGACCAGTCAAC |
| SEQ ID NO: 43 | HPRT | HPRT-6 | GATGATCTCTCAACTTTAAC |
| SEQ ID NO: 44 | FactorVIII | FactorVIII-1 | CACTAAAGCAGAATCGCAAA |
| SEQ ID NO: 45 | FactorVIII | FactorVIII-2 | TGCCTTTACCTTGCGTCCAC |
| SEQ ID NO: 46 | FactorVIII | FactorVIII-3 | CCTGTCAGTCTTCATGCTGT |
| SEQ ID NO: 47 | FactorVIII | FactorVIII-4 | TCTGCTAGGTCCTACCATCC |
| SEQ ID NO: 48 | FactorIX | FactorIX-1 | CTTTCACAATCTGCTAGCAA |
| SEQ ID NO: 49 | FactorIX | FactorIX-2 | AAATTCTGAATCGGCCAAAG |
| SEQ ID NO: 50 | FactorIX | FactorIX-3 | CGGCCAAAGAGGTATAATTC |
| SEQ ID NO: 51 | FactorIX | FactorIX-4 | ATTCTTTATAGACTGAATTT |
| SEQ ID NO: 52 | LRRK2 | LRRK2-1 | GCTCAGTACTGCTGTAGAAT |
| SEQ ID NO: 53 | LRRK2 | LRRK2-2 | TGCTCAGTACTGCTGTAGAA |
| SEQ ID NO: 54 | HTT | HTT-1 | GAAGGACTTGAGGGACTCGA |
| SEQ ID NO: 55 | HTT | HTT-2 | AGCGGCTGTGCCTGCGGCGG |
| SEQ ID NO: 56 | HTT | RHO-1 | GCGTACCACACCCGTCGCAT |
| SEQ ID NO: 57 | HTT | RHO-2 | CGAGTACCCACAGTACTACC |
| SEQ ID NO: 58 | HTT | RHO-3 | CCTGTGGTCCTTGGTGGTCC |
| SEQ ID NO: 59 | CTFR | CTFR-1 | ATATTTTCTTTAATGGTGCC |
| SEQ ID NO: 60 | CTFR | CTFR-2 | TCTGTATCTATATTCATCAT |
| SEQ ID NO: 61 | SFTPB | SFTPB-1 | GTGGTACCTCTGGTGGCGGG |
| SEQ ID NO: 62 | SFTPB | SFTPB-2 | GCTAGCTGTGGCAGTGGCCC |
| SEQ ID NO: 63 | PD1 | PD1-1 | GAAGGTGGCGTTGTCCCCTT |
| SEQ ID NO: 64 | PD1 | PD1-2 | ATGTGGAAGTCACGCCCGTT |
| SEQ ID NO: 65 | CTLA-4 | CTLA4-1 | CCTTGGATTTCAGCGGCACA |
| SEQ ID NO: 66 | CTLA-4 | CTLA4-2 | TGCATACTCACACACAAAGC |
| SEQ ID NO: 67 | CTLA-4 | CTLA4-3 | AGCTGTTTCTTTGAGCAAAA |
| SEQ ID NO: 68 | HLA-A | HLA-A-1 | CGGCTCCATCCTCTGGCTCG |
| SEQ ID NO: 69 | HLA-A | HLA-A-2 | CCTTCACATTCCGTGTCTCC |
| SEQ ID NO: 70 | HLA-A | HLA-A-3 | CCTGCGCTCTTGGACCGCGG |
| SEQ ID NO: 71 | HLA-A | HLA-A-4 | CTGAGCCGCCATGTCCGCCG |
| SEQ ID NO: 72 | HLA-B | HLA-B-1 | GCAGGAGGGGCCGGAGTATT |
| SEQ ID NO: 73 | HLA-B | HLA-B-2 | TGGACGACACCCAGTTCGTG |
| SEQ ID NO: 74 | HLA-B | HLA-B-3 | CTCTCCGCTGCTCCGCCTCA |
| SEQ ID NO: 75 | HLA-B | HLA-B-4 | GATCTGAGCCGCCGTGTCCG |
| SEQ ID NO: 76 | HLA-C | HLA-C-1 | GTAGAACAAAAAAAAAGACC |
| SEQ ID NO: 77 | HLA-C | HLA-C-2 | TGGGCACTGTTGCTGVCTGG |
| SEQ ID NO: 78 | HLA-C | HLA-C-3 | GAGAGACTCATCAGAGCCCT |
| SEQ ID NO: 79 | HLA-C | HLA-C-4 | CTTCCTCCTACACATCATAG |
| SEQ ID NO: 80 | HLA-C | HLA-C-5 | TAGCGGTGACCACAGCTCCA |
| SEQ ID NO: 81 | HLA-DPA | HLA-DPA-1 | GAAGGAGACCGTCTGGCATC |
| SEQ ID NO: 82 | HLA-DPA | HLA-DPA-2 | TCAAACATAAACTCCCCTGT |
| SEQ ID NO: 83 | HLA-DPA | HLA-DPA-3 | AATCTGTTCTGGGCAGGAAG |
| SEQ ID NO: 84 | HLA-DPA | HLA-DPA-4 | CCCTGCAGTCATAGAAGTCC |
| SEQ ID NO: 85 | HLA-DQ | HLA-DQ-1 | TGTGGAGGTGAAGACATTGT |
| SEQ ID NO: 86 | HLA-DQ | HLA-DQ-2 | TCGCTCTGACCACCGTGATG |
| SEQ ID NO: 87 | HLA-DRA | HLA-DRA-1 | TGTGGAACTGAGAGAGCCCA |
| SEQ ID NO: 88 | HLA-DRA | HLA-DRA-2 | CCAGTACCTCCAGAGGTAAC |
| SEQ ID NO: 89 | HLA-DRA | HLA-DRA-3 | GATGAGCGCTCAGGAATCAT |
| SEQ ID NO: 90 | LMP-7 | LMP-7-1 | GCCACTGTCCATGACCCCGT |
| SEQ ID NO: 91 | LMP-7 | LMP-7-2 | GTGGAGAACATATTTCCTGA |
| SEQ ID NO: 92 | LMP-7 | LMP-7-3 | TGGGCCATCTCAATCTGAAC |
| SEQ ID NO: 93 | LMP-7 | LMP-7-4 | TGCTGGAACTTGAAGGCGAG |
| SEQ ID NO: 94 | TAP1 | TAP1-1 | TCATCCAGGATAAGTACACA |
| SEQ ID NO: 95 | TAP1 | TAP1-2 | GATCAATGCTCGGGCCAACG |
| SEQ ID NO: 96 | TAP1 | TAP1-3 | ACGCCACTGCCTGTCGCTGA |
| SEQ ID NO: 97 | TAP2 | TAP2-1 | TGAGGAAGCAAAGTCCCCAG |
| SEQ ID NO: 98 | TAP2 | TAP2-2 | AGCCGCGTCCACCAGCAGCA |
| SEQ ID NO: 99 | TAPBP | TAPBP-1 | TCCTGAAAGGGTTGAACTGT |
| SEQ ID NO: 100 | TAPBP | TAPBP-2 | TTTCCGGTCCATGGGCCCCA |
| SEQ ID NO: 101 | CUTA | CUTA-1 | CTCGGGGTAGCAACAAAAGG |
| SEQ ID NO: 102 | CUTA | CUTA-2 | GCCATGGTCAGCAAGACTCG |
| SEQ ID NO: 103 | DMD | DMD-1 | TGGCAAAGTCTCGAACATCT |
| SEQ ID NO: 104 | DMD | DMD-2 | ATTCGGGGATGCTTCGCAAA |
| SEQ ID NO: 105 | DMD | DMD-3 | CTATTATGAAGAATCAAAGC |
| SEQ ID NO: 106 | DMD | DMD-4 | CAGTTTTAAAAGACAGGACA |
| SEQ ID NO: 107 | GR/NR3C1 | NR3C1-1 | CCTGAGCAAGCACACTGCTG |
| SEQ ID NO: 108 | IL2RG | IL2RG-1 | CTAGGTTCTTCAGGGTGGGA |
| SEQ ID NO: 109 | IL2RG | IL2RG-2 | GTCCTGACAGGGGAGAAAGA |
| SEQ ID NO: 110 | IL2RG | IL2RG-3 | TTAGGTTCTCTGGAGCCCAG |
| SEQ ID NO: 111 | IL2RG | IL2RG-4 | GTTAGGTTCTCTGGAGCCCA |
| SEQ ID NO: 112 | RFX5 | RFX5-1 | AAGGATACTTGGACTGGCCC |
| SEQ ID NO: 113 | RFX5 | RFX5-2 | TCGAGCTTTGATGTCAGGAA |
| SEQ ID NO: 114 | AR/NR3C4 | NR3C4-1 | ACAGGCTACCTGGTCCTGGA |
| SEQ ID NO: 115 | AR/NR3C4 | NR3C4-2 | TCTCCCCAAGCCCATCGTAG |
| SEQ ID NO: 116 | AR/NR3C4 | NR3C4-3 | ACTCTCTTCACAGCCGAAGA |
| SEQ ID NO: 117 | AR/NR3C4 | NR3C4-4 | TAGCCCCCTACGGCTACACT |
| SEQ ID NO: 118 | AR/NR3C4 | NR3C4-5 | AAGATCCTTTCTGGGAAAGT |
| SEQ ID NO: 119 | AR/NR3C4 | NR3C4-6 | CATGGTGAGCGTGGACTTTC |
| SEQ ID NO: 120 | TGFBR1 | TGFBR1-1 | TTGCTTGTTCAGAGAACAAT |
| SEQ ID NO: 121 | TGFBR1 | TGFBR1-2 | ATTGTGTTACAAGAAAGCAT |

### HDR Template Sequence

Genome stability necessitates the correct and efficient repair of DSBs. In eukaryotic cells, mechanistic repair of DSBs occurs primarily by two pathways: Non-Homologous End-Joining (NHEJ) and Homology Directed Repair (HDR). NHEJ is the canonical homology-independent pathway as it involves the alignment of only one to a few complementary bases at most for the re-ligation of two ends, whereas HDR uses longer stretches of sequence homology to repair DNA lesions. HDR is the more accurate mechanism for DSB repair due to the requirement of higher sequence homology between the damaged and intact donor strands of DNA. The process is error-free if the DNA template used for repair is identical to the original DNA sequence at the DSB, or it can introduce very specific mutations into the damaged DNA.

As addressed above, HDR methods provide the great freedom in genomic engineering, allowing for as little as single base mutations and up to insertions or deletions of kilo-bases (kb) of DNA. In eukaryotes, HDR rate is governed by the competition between two different pathways: Homologous Recombination (HR) and Non-Homologous End Joining (NHEJ). The competition between these two pathways begins by competitive binding by either MRN/CtIP complex or Ku 70/80 heterodimer. If MRN/CtIP bind first, they recruit other proteins, including Exonuclease I (ExoI), which possess 5′->3′ exonuclease activity20. 5′ end resection of double strand DNA breaks by either ExoI or Dna2 at each side of the break commits the DSB to be repaired by the HR pathway. Alternatively, if the Ku 70/80 heterodimer binds, it can then recruit other NHEJ pathway members, including DNA Ligase IV, and eventually repairs the double strand break via NHEJ.

HDR template sequences are needed to be delivered into cells when delivering the CRISPR-Cas9 system to the cells. HDR templates used to create specific mutations or insert new elements into a gene require a certain amount of homology surrounding the target sequence that will be modified. In some embodiments, the 5′ and 3′ homology arms start at the CRISPR-induced DSB. In general, the insertion sites of the modification can be very close to the DSB, ideally less than 10 bp away if possible. In some embodiments, the 5′ and 3′ homology arm of the HDR template sequences are at least 80% identical to the targeted sequence. Further, in some embodiments, single stranded donor oligonucleotide (ssDON) is utilized for smaller insertions. Each homology arm of the ssDON may comprise about 30-80 bp nucleotide sequence. The length of the homology arm is not meant to be limiting and the length can be adjusted by a person of ordinary skill in the art according to a locus of gene interest and experimental system. For larger insertions such as fluorescent proteins or selection cassettes, double stranded donor oligonucleotide (dsDON) can be utilized as HDR template sequence. In some embodiments, each homology arm of the ssDON may comprise about 800-1500 bp nucleotide sequence. To prevent Cas9 enzyme cleaving the HDR template, in some embodiments, a single base mutation can be introduced in the Protospacer Adjacent Motif (PAM) sequence of the HDR template.

### Methods for Delivery

Several different methods are used to deliver ribonucleoproteins and ssDON or other nucleic acids to a cleavage site, such as transfection. Transfection methods can be used to deliver CRISPR-Cas9 or other programmable endonuclease components to cells. Some of exemplary methods can be used to deliver the disclosed modified CRISPR-Cas9 system to cells and additional methods consistent with the disclosure known to a person of ordinary skill in the art can choose a particular method depending on the type of cells and the format of the CRISPR-Cas9 components.

Delivery can be broken into two major categories: cargo and delivery vehicle. Regarding CRISPR/Cas9 cargoes, three approaches are commonly available: (1) DNA plasmid encoding both the Cas9 protein or other programmable endonuclease and the guide RNA, (2) mRNA for Cas9 or other programmable endonuclease translation alongside a separate guide RNA, and (3) Cas9 protein or other programmable endonuclease with guide RNA (ribonucleoprotein complex). The delivery vehicle used will often dictate which of these three cargos can be packaged, and whether the system is usable in vitro and/or in vivo.

Vehicles used to deliver the gene editing system cargo can be classified into three general groups: physical delivery, viral vectors, and non-viral vectors. The most common physical delivery methods are microinjection, electroporation, and nucleofection. Electroporation enables delivery of the CRISPR machinery in cell types that are difficult to transform using lipid-based delivery systems. Application of a controlled, short electric pulse to the cells forms pores in the cell membrane, allowing entry of foreign material. Nucleofection is a variant of electroporation, in which the electric pulse is optimized such that the nuclear membrane of the cells also forms pores. The CRISPR components are thus directly delivered inside the nucleus. Microinjection is commonly used to inject the Cas9 or other programmable endonuclease and gRNA ribonucleoprotein complex in embryos, although it can also be used in cells. Zebrafish, mouse, and most recently human embryos have been manipulated using this technique.

Viral delivery vectors include specifically engineered adeno-associated virus (AAV), and full sized adenovirus and lentivirus vehicles. Especially for in vivo work, viral vectors have found favor and are the most common CRISPR/Cas9 delivery vectors. AAV, of the Dependovirus genus and Parvoviridae family, is a single stranded DNA virus that has been extensively utilized for gene therapy. While LVs and AdVs are clearly distinct, the way they are utilized for delivery of CRISPR/Cas9 components is quite similar. In the case of LV delivery, the backbone virus is a provirus of HIV; for AdV delivery, the backbone virus is one of the many different serotypes of known AdVs. Both LV and AdV can infect dividing and non-dividing cells; however, unlike LV, AdV does not integrate into the genome. This is advantageous in the case of CRISPR/Cas9-based editing for limiting off-target effects. As is the case with AAV particles, both LV and AdV can be used in in vitro, ex vivo, and in vivo applications, which eases both efficacy and safety testing. In terms of mechanism, this class of CRISPR/Cas9 delivery is like AAV delivery described above. Full viral particles containing the desired Cas9 and sgRNA are created via transformation of HEK 293 T cells. These viral particles are then used to infect the target cell type. The biggest difference between LV/AdV delivery and AAV delivery is the size of the particle; both LVs and AdVs are roughly 80-100 nm in diameter. Compared with the 20 nm diameter of AAV, larger insertions are better tolerated in these systems. When considering CRISPR/Cas9, additional packaging space for differently-sized Cas9 constructs or several sgRNAs for multiplex genome editing is a significant advantage over the AAV delivery system.

A viral vector can be a modified viral vector, alternatively, it can be an unmodified vector. Often, the modified viral vector is a genetically modified vector. The modified viral vector can show reduced immunogenicity, an increase in the persistence of the vector in the blood stream, or impaired uptake of the vector by macrophages and antigen presenting cells.

The modified viral vector can further comprise a polymer, a lipid, a peptide, a magnetic nanoparticle (MNP), an additional compound, or a combination thereof. The polymer, lipid, or magnetic nanoparticle can be attached to a capsid of the viral vector. The polymer can be a polyethylene glycol (PEG). The polymer can be N-[2-hydroxypropyl] methacrylamide (HPMA), poly(2-(dimethylamino)ethyl methacrylate) (pDMAEMA), or arginine-grafted bioreducible polymers (ABPs). The peptide can be a cell-penetrating peptide, a cell adhesion peptide, or a peptide which binds to a receptor on a cell. The cell can be a tumor cell. Any suitable cell-penetrating peptide can be used. Examples of cell-penetrating peptides include, but are not limited to a polylysine peptide and a polyarginine peptide. The cell adhesion peptide can be an arginylglycylaspartic acid (RGD) peptide. An additional compound can be a compound which binds to a receptor on a cell, such as folic acid.

In some instances, the modified viral vector is a genetically modified vector. The genetically modified vector can have reduced immunogenicity, reduced genotoxicity, increased loading capacity, increased transgene expression, or a combination thereof. In some instances, the genetically modified viral vector is a pseudotyped viral vector. The pseudotyped viral vector can have at least one foreign viral envelope protein. The foreign viral envelope protein can be an envelope protein from a lyssavirus, an arenavirus, a hepadnavirus, a flavivirus, a paramyxovirus, a baculovirus, a filovirus, or an alphavirus. The foreign viral envelope protein can be the glycoprotein G of a vesicular stomatitis virus (VSV). In some instances, the foreign viral envelope protein is a genetically modified viral envelope protein. The genetically modified viral envelope protein can be a non-naturally occurring viral envelope protein.

In some embodiments, the viral vectors are virus-like particles (VLPs). VLPs resemble viruses but are non-infectious because they do not contain viral genetic materials. VLPs have been produced from components of a wide variety of virus families including Parvoviridae (e.g. adeno-associated virus), Retroviridae (e.g. HIV), Flaviviridae (e.g. Hepatitis C virus) and bacteriophages. VLPs can be produced in multiple cell culture systems including bacteria, mammalian cell lines, insect cell lines, yeast and plant cells.

With respect to non-viral vector delivery vehicles, lipid nanoparticles/liposomes can be used herein. A lipid can be a cationic lipid, an anionic lipid, or neutral lipid. The lipid can be a liposome, a small unilamellar vesicle (SUV), a lipidic envelope, a lipidoid, or a lipid nanoparticle (LNP). The lipid can be mixed with the nucleic acid to form a lipoplex (a nucleic acid-liposome complex). The lipid can be conjugated to the nucleic acid. The lipid can be a non-pH sensitive lipid or a pH-sensitive lipid. The lipid can further comprise a polythethylene glycol (PEG).

The cationic lipid can be a monovalent cationic lipid, such as N-[1-(2,3-dioleyloxy)propyl]-N,N,N-trimethylammonium chloride (DOTMA), [1,2-bis(oleoyloxy)-3-(trimethylammonio)propane] (DOTAP), or 3β[N-(N′, N′-dimethylaminoethane)-carbamoyl] cholesterol (DC-Chol). The cationic lipid can be a multivalent cationic lipid, such as Di-octadecyl-amido-glycyl-spermine (DOGS) or {2,3-dioleyloxy-N-[2(sperminecarboxamido)ethyl]-N,N-dimethyl-1-propanaminium trifluoroacetate} (DOSPA).

The anionic lipid can be a phospholipid or dioleoylphosphatidylglycerol (DOPG). Examples of phospholipids include, but are not limited to, phosphatidic acid, phosphatidylglycerol, or phosphatidylserine. In some instances, the anionic lipid further comprises a divalent cation, such as Ca²⁺, Mg²⁺, Mn²⁺, and Ba²⁺.

The cationic lipid or the anionic lipid can further comprise a neutral lipid. The neutral lipid can be dioleoylphosphatidyl ethanolamine (DOPE) or dioleoylphosphatidylcholine (DOPC). In some instances, the use of a helper lipid in combination with a charged lipid yields higher transfection efficiencies.

The liposome can further comprise a polymer, a lipid, a peptide, a magnetic nanoparticle (MNP), an additional compound, or a combination thereof. The polymer, lipid, or magnetic nanoparticle can be attached to the liposome or integrated into the liposomal membrane. The polymer can be a polyethylene glycol (PEG). The polymer can be N-[2-hydroxypropyl] methacrylamide (HPMA), poly (2-(dimethylamino)ethyl methacrylate) (pDMAEMA), or arginine-grafted bioreducible polymers (ABPs). The peptide can be a cell-penetrating peptide, a cell adhesion peptide, or a peptide which binds to a receptor on a cell. The cell can be a tumor cell. Any suitable cell-penetrating peptide can be used. Examples of cell-penetrating peptides include, but are not limited to a polylysine peptide and a polyarginine peptide. The cell adhesion peptide can be an arginylglycylaspartic acid (RGD) peptide. An additional compound can be a compound which binds to a receptor on a cell, such as folic acid.

### Kit

Disclosed herein are kits and articles of manufacture for use with one or more methods and compositions described herein. The kit can comprise a polynucleotide composition described herein formulated in a compatible pharmaceutical excipient and placed in an appropriate container.

The kit can include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. A container can be formed from a variety of materials such as glass or plastic.

The kit can include an identifying description, a label, or a package insert. The label or package insert can list contents of kit or the immunological composition, instructions relating to its use in the methods described herein, or a combination thereof. The label can be on or associated with the container. The label can be on a container when letters, numbers, or other characters forming the label are attached, molded or etched into the container itself. The label can be associated with a container when it is present within a receptacle or carrier that also holds the container, e.g., as a package insert. In some instances, the label is used to indicate that the contents are to be used for a specific therapeutic application.

The kit herein can further comprise one or more reagents that used to deliver the polynucleotide sequences to cells, tissues, or organs.

### Applications

The disclosed RNPs can be introduced into cells using one of the delivery methods disclosed herein to induce homologues recombination of DNA in the cells. Further, the disclosed RNPs can be introduced into cells using one of the delivery methods disclosed herein to induce HDR in cells in vitro or ex vivo. The DNA molecule is contacted with the RNPs. The modified Cas9 protein guided by a gRNA introduces a DSB by cleaving at a location as determined by the hybridization of the gRNA with the DNA molecule. The hExo1peptide partially digests the cleaved DNA molecule, leaving a 3′ or 5′ overhang. The HDR template sequences comprising some degrees of sequence homology as the digested DNA molecule promotes and serves as the template for HDR. After HDR, the DNA molecule in the cell comprises a sequence that is identical to the HDR template at the region where homologous recombination occurs.

By inducing HDR in cells, the cellular toxicity caused by wild type Cas9 protein along with gRNAs is decreased. Cellular toxicity can be measured by several cell viability assays. In some embodiments, tetrazolium reduction assay is used. A variety of tetrazolium compounds have been used to detect viable cells. The most commonly used compounds include: MTT, MTS, XTT, and WST-1. These compounds fall into two basic categories: 1) MTT which is positively charged and readily penetrates viable eukaryotic cells and 2) those such as MTS, XTT, and WST-1 which are negatively charged and do not readily penetrate cells. The latter class (MTS, XTT, WST-1) are typically used with an intermediate electron acceptor that can transfer electrons from the cytoplasm or plasma membrane to facilitate the reduction of the tetrazolium into the colored formazan product. For example, viable cells with active metabolism convert MTT into a purple colored formazan product with an absorbance maximum near 570 nm. When cells die, they lose the ability to convert MTT into formazan, thus color formation serves as a useful and convenient marker of only the viable cells.

In other embodiments, resazurin reduction assay is used. Resazurin is a cell permeable redox indicator that can be used to monitor viable cell number with protocols similar to those utilizing the tetrazolium compounds. Resazurin can be dissolved in physiological buffers (resulting in a deep blue colored solution) and added directly to cells in culture in a homogeneous format. Viable cells with active metabolism can reduce resazurin into the resorufin product which is pink and fluorescent. The quantity of resorufin produced is proportional to the number of viable cells which can be quantified using a microplate fluorometer equipped with a 530 nm or 560 nm excitation/590 nm emission filter set. The wavelength can be adjusted according to different types of cells and experimental designs. Resorufin also can be quantified by measuring a change in absorbance; however, absorbance detection is not often used because it is far less sensitive than measuring fluorescence.

Further, the disclosed RNPs herein are used to treat diseases where the causes of the diseases are tranced to a locus of chromosomal abnormality. In certain embodiments, a biological sample is obtained from a subject afflicted with a disease. DNA is extracted from the biological sample and sequenced to determine the locus of chromosomal abnormality. Primary cells harboring the chromosomal abnormality are isolated from the subject and cultured ex vivo. The RNPs are delivered into the said cultured primary cells using one of the delivery methods disclosed herein. The HDR template sequences are also delivered into the cultured primary cells. In some embodiments, the gRNA moiety comprises at least 10 nucleotides complementary to the targeted locus of chromosomal abnormality. The HDR template sequences comprise an integration cassette flanked by a 5′ homology region and a 3′ homology region, wherein the 5′ homology region and the 3′ homology region exhibit at least 80% identity to adjacent segments of the targeted locus. The integration cassette of the HDR template comprises a wild type sequence that corresponds to the locus of chromosomal abnormality as detected in the primary cells. Upon delivering of the RNPs, the gRNA directs the protein fusion complex to the targeted locus, where the modified Cas protein moiety creates a DSB by cleaving said targeted locus as recognized by the gRNA. The nuclease moiety partially digests the cleaved locus of chromosomal abnormality, leaving a 3′ overhang. The presence of the HDR template sequences promotes endogenous repair through HDR. Primary cells with wild type sequence replacing chromosomal abnormality are screened and selected for reintroducing back into the subject.

In some embodiments, primary cells are selected from the group comprising T cells, B cells, dendritic cells, natural killer cells, natural killer cells, macrophages, neutrophils, eosinophils, basophils, mast cells, hematopoietic progenitor cells, hematopoietic stem cells (HSCs), red blood cells, blood stem cells, endoderm stem cells, endoderm progenitor cells, endoderm precursor cells, differentiated endoderm cells, mesenchymal stem cells (MSCs), mesenchymal progenitor cells, mesenchymal precursor cells, differentiated mesenchymal cells, hepatocytes progenitor cells, pancreatic progenitor cells, lung progenitor cells, tracheae progenitor cells, bone cells, cartilage cells, muscle cells, adipose cells, stromal cells, fibroblasts, and dermal cells.

Further, in some embodiments, the gRNA is configured to recognize exon 1 of the human HBB gene. The HDR template is configured to have 5′ and 3′ arm homology with a functional human HBB gene. In other embodiments, the gRNA is configured to recognize a region of CFTR and the HDR template is designed to have 5′ and 3′ arm homology with a functional CFTR gene.

Please see a list of single gene disorders with the mutated locus of gene respectively listed in Table 3. Examples of human monogenic diseases, modes of inheritance, and associated genes.

### Table 3.

TABLE 3

| Disease | Type of Inheritance | Gene Responsible |
|---|---|---|
| Phenylketonuria (PKU) | Autosomal recessive | Phenylalanine hydroxylase (PAH) |
| Cystic fibrosis | Autosomal recessive | Cystic fibrosis conductance |
| | | transmembrane regulator (CFTR) |
| Sickle-cell anemia | Autosomal recessive | Beta hemoglobin (HBB) |
| Albinism, oculocutaneous, | Autosomal recessive | Oculocutaneous albinism II (OCA2) |
| type II | | |
| Glucocorticoid Resistance | Autosomal dominant | Glucocorticoid Receptor (GR) |
| Syndrome | | |
| Huntington's disease | Autosomal dominant | Huntingtin (HTT) |
| Myotonic dystrophy type 1 | Autosomal dominant | Dystrophia myotonica-protein kinase |
| | | (DMPK) |
| Hypercholesterolemia, | Autosomal dominant | Low-density lipoprotein receptor |
| autosomal dominant, type B | | (LDLR); apolipoprotein B (APOB) |
| Neurofibromatosis, type 1 | Autosomal dominant | Neurofibromin 1 (NF1) |
| Polycystic kidney disease 1 | Autosomal dominant | Polycystic kidney disease 1 (PKD1) |
| and 2 | | and polycystic kidney disease 2 |
| | | (PKD2), respectively |
| Hemophilia A | X-linked recessive | Coagulation factor VIII (F8) |
| Hemophilia B | X-linked recessive | Coagulation factor IX (F9) |
| LRRK2 Linked Parkinson's | Autosomal Dominant | Leucine-Rich Repeat Kinase |
| Disease | | 2(LRRK2) |
| Muscular dystrophy, | X-linked recessive | Dystrophin (DMD) |
| Duchenne type | | |
| Pulmonary Surfactant | Autosomal Recessive | SFTB-B, ABCA3 |
| Metabolism Disorder 1 | | |
| Hypophosphatemic rickets, | X-linked dominant | Phosphate-regulating endopeptidase |
| X-linked dominant | | homologue, X-linked (PHEX) |
| Rett's syndrome | X-linked dominant | Methyl-CpG-binding protein 2 |
| | | (MECP2) |
| Spermatogenic failure, | Y-linked | Ubiquitin-specific peptidase 9Y, Y- |
| nonobstructive, Y-linked | | linked (USP9Y) |
| X-linked severe combined | X-linked recessive | Interleukin 2 receptor subunit gamma |
| immunodeficiency (XSCID) | | (IL2RG) |

Moreover, the disclosed RNPs herein are used to introduce genetic modification to confer immunity against diseases. A biological sample is obtained from a subject. DNA is extracted and the locus for the targeted genetic modification is sequenced. Primary cells the subjected are isolated and cultured ex vivo. RNPs and the HDR template sequences are delivered into said cultured primary cells. The gRNA moiety directs the RNPs to the targeted locus to initiate the formation of DSB and DNA digestion to generate the 3′ overhang. The HDR template comprises an integration cassette flanked by a 5′ homology region and a 3′ homology region, wherein the 5′ homology region and the 3′ homology region exhibit at least 80% identity to adjacent segments of the targeted loci. The integration cassette comprises a wild type sequence that is different from the subject's sequence at the targeted locus. The presence of the polynucleotide promotes endogenous repair through HDR. Primary cells harboring wild type sequence encoded by the polynucleotide are screened and selected for reintroducing back into the subject.

### Certain Definitions

As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “polypeptide,” “peptide” and “protein” are often used interchangeably herein in reference to a polymer of amino acid residues. A protein, generally, refers to a full-length polypeptide as translated from a coding open reading frame, or as processed to its mature form, while a polypeptide or peptide informally refers to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

As used herein, the terms “fragment,” “domain,” or equivalent terms refer to a portion of a protein that has less than the full length of the protein and maintains the function of the protein. Further, when the portion of the protein is blasted again the protein, the portion of the protein sequence would align at least with 80% identity to part of the protein sequence.

As used herein, the terms “polynucleotide,” “nucleic acid,” “oligonucleotide,” or equivalent terms, refer to molecules that comprises a polymeric arrangement of nucleotide base monomers, where the sequence of monomers defines the polynucleotide. Polynucleotides can include polymers of deoxyribonucleotides to produce deoxyribonucleic acid (DNA), and polymers of ribonucleotides to produce ribonucleic acid (RNA). A polynucleotide can be single or double stranded. When single stranded, the polynucleotide can correspond to the sense or antisense strand of a gene. A single-stranded polynucleotide can hybridize with a complementary portion of a target polynucleotide to form a duplex, which can be a homoduplex or a heteroduplex. The length of a polynucleotide is not limited in any respect. Linkages between nucleotides can be internucleotide-type phosphodiester linkages, or any other type of linkage. A polynucleotide can be produced by biological means (e.g., enzymatically), either in vivo (in a cell) or in vitro (in a cell-free system). A polynucleotide can be chemically synthesized using enzyme-free systems. A polynucleotide can be enzymatically extendable or enzymatically non-extendable.

As used herein, the terms “vector,” “vehicle,” “construct” and “plasmid” are used in reference to any recombinant polynucleotide molecule that can be propagated and used to transfer nucleic acid segment(s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contain operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “gene” generally refers to a combination of polynucleotide elements, that when operatively linked in either a native or recombinant manner, provide some product or function. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” encompasses mRNA, cDNA and genomic forms of a gene.

As used herein, the terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cancer. In some cases, the subject is not necessarily diagnosed or suspected of being at high risk for the disease.

As used herein, the term “in vivo” is used to describe an event that takes place in a subject's body.

As used herein, the term “ex vivo” is used to describe an event that takes place outside of a subject's body. An “ex vivo” assay is not performed on a subject. Rather, it is performed upon a sample separate from a subject. An example of an ‘ex vivo’ assay performed on a sample is an ‘in vitro’ assay.

As used herein, the term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

Certain ranges are presented herein with numerical values being preceded by the term “about.” The term “about” is used herein to provide literal support for the exact number that it precedes, as well as a number that is near to or approximately the number that the term precedes, such as a number that is within 10% of the value of the number that it precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

Mention is frequently made to “Cas9” throughout the disclosure. It is understood that, although Cas9 is a particular embodiment, additional programmable endonucleases are also contemplated, such as Cas12 or others. Accordingly, mention of Cas9 should not always be read to exclude alternate or other programmable endonuclease.

Similarly, “hEXO1” is frequently referred to. It is understood that, although hEXO lis a particular embodiment, additional programmable endonucleases are also contemplated. Accordingly, mention of hEXO1 should not always be read to exclude alternate or other exonuclease.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### FIGURE DESCRIPTIONS

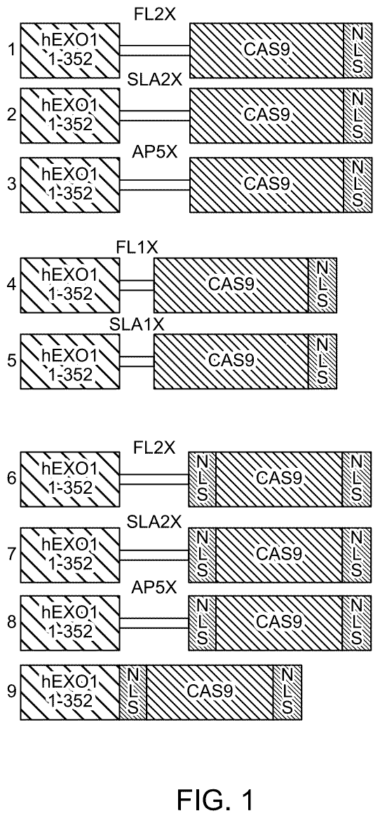

FIG. 1 shows 9 Cas9-HR fusion proteins. The first fusion protein comprises a hExo1(SEQ ID NO:1) coupled to a Cas9 (anyone of SEQ ID NO:2-18) with a N-terminal NLS via linker FL2X. The second fusion protein comprises a hExo1(SEQ ID NO:1) coupled to a Cas9 (anyone of SEQ ID NO:2-18) with a N-terminal NLS via linker SLA2X. The third fusion protein comprises a hExo1(SEQ ID NO:1) coupled to a Cas9 (anyone of SEQ ID NO:2-18) with a N-terminal NLS via linker AP5X. The fourth fusion protein comprises a hExo1(SEQ ID NO:1) coupled to a Cas9 (anyone of SEQ ID NO:2-18) with a N-terminal NLS via linker FL1X. The fifth fusion protein comprises a hExo1 (SEQ ID NO:1) coupled to a Cas9 (anyone of SEQ ID NO:2-18) with a N-terminal NLS via linker SLA1X. The sixth fusion protein comprises a hExo1(SEQ ID NO:1) coupled to a Cas9 (anyone of SEQ ID NO:2-18) with a N-terminal NLS and a C-terminal NLS via linker FL2X. The seventh fusion protein comprises a hExo1 (SEQ ID NO:1) coupled to a Cas9 (anyone of SEQ ID NO:2-18) with a N-terminal NLS and a C-terminal NLS via linker SLA2X. And the eight fusion protein comprises a hExo1(SEQ ID NO:1) coupled to a Cas9 (anyone of SEQ ID NO:2-18) with a N-terminal NLS and a C-terminal NLS via linker AP5X. The ninth fusion protein compromises a hExo1(SEQ ID NO:1) directly coupled to a Cas9 (anyone of SEQ ID NO:2-18) with a N-terminal NLS and C-terminal NLS.

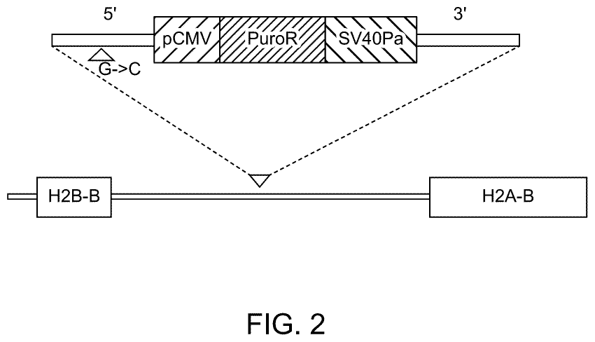

FIG. 2 shows an embodiment of an intended target site for nucleotides cleaving and replacing. The intended target site is about 1 kb to the 3′ end of the human H2B gene on chromosome 6. This figure also shows an embodiment of a HDR template, which contains a puromycin antibiotic resistance cassette coupled to a CMV promoter at the 5′ end and coupled to SV40 poly(A) at the 3′ end. Further, the HDR template contains 5′ and 3′ homology regions to the intended target site described above. A single G->C mutation introduced in the PAM sequence to prevent RNP cutting of the HDR template.

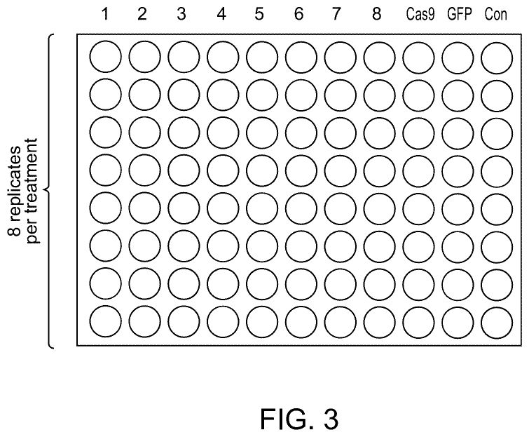

FIG. 3 shows an embodiment of experiment design. Cells are cultured in a 96-well plate and each well is seeded with about 2.5×10⁴ cells. Each column of the 96-well plate receives a treatment with different plasmid respectively. Because each column contains 8 wells, each treatment has 8 replicates. The cells in the first column are transfected with plasmids encoding the first fusion protein as shown in FIG. 1; the cells in the second column with plasmids encoding the second fusion protein as shown in FIG. 1; the cells in the third column with plasmids encoding the third fusion protein as shown in FIG. 1; the cells in the fourth column with plasmids encoding the fourth fusion protein as shown in FIG. 1; the cells in the fifth column with plasmids encoding the fifth fusion protein as shown in FIG. 1; the cells in the sixth column with plasmids encoding the sixth fusion protein as shown in FIG. 1; the cells in the seventh column with plasmids encoding the seventh fusion protein as shown in FIG. 1; and the cells in the eighth column with plasmids encoding the eighth fusion protein as shown in FIG. 1. Further, the cells in the ninth column are transfected with plasmids encoding unmodified Cas9 enzymes. The cells in the tenth column are transfected with plasmids encoding GFPs. The cells in the eleventh column are controls without any treatment.

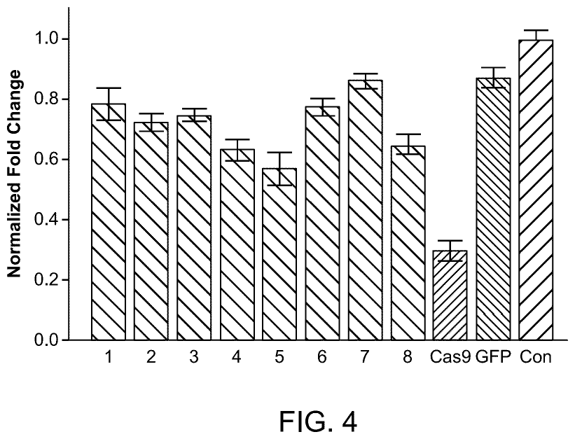

FIG. 4 shows a bar graph displaying normalized fold changes of the measured resorufin fluorescence before puromycin selection. The y-axis displays numbers of normalized fold change and the x-axis displays treatments with plasmids encoding the 8 fusion proteins respectively, unmodified Cas9, and GFP and non-transfected controls. It is expected that cells from the control treatment to have the greatest resorufin fluorescence due to minimal cellular toxicity. The control treatment's resorufin fluorescence measurement is normalized to 1, accordingly, their normalized fold change number is 1. Every other treatment's resorufin fluorescence measurement is compared to the control treatment to obtain the normalized fold change number. It is also expected that all the treatments have some degree of cellular toxicity, therefore, each treatment can have a normalized fold change number smaller than 1. For example, the treatment with wild type Cas9 displays the smallest fold change number compared to the control treatment, which means that the wild type Cas9 transfected cells have the least amount of resorufin fluorescence. In contrast, treatments with plasmids encoding the seventh fusion protein and GFP have similar and the largest fold change number, which indicates that that the transfected cells have the second greatest amount of resorufin fluorescence.

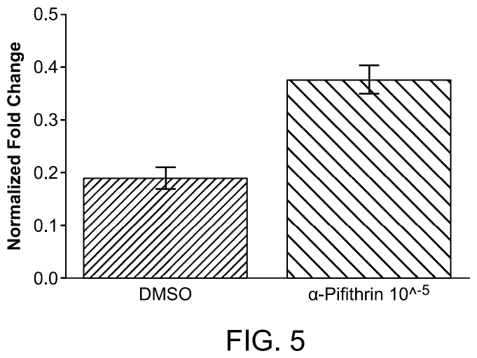

FIG. 5 shows a bar graph displaying normalized fold change of the measured resorufin fluorescence at day 2 after the cells are transfected with plasmids encoding wild type Cas9 enzymes. The left bar displays a normalized fold change number of cells treated with DMSO and the right bar displays a normalized fold change number of cells treated with PFT-α, which specifically block transcriptional activity of the tumor suppressor p53. The right bar displays a higher number than the left bar, which means the cells treated with PFT-α have increased resorufin fluorescence measurements, therefore PFT-α reduces cellular toxicity. This indicates that the cellular toxicity associated with CRISPR-Cas9 system seen in A549 cells is at least partially dependent on p53, which is the main factor driving Cas9 mediated cellular toxicity seen in other human cell types. A549 cells are positive for p53 activity.

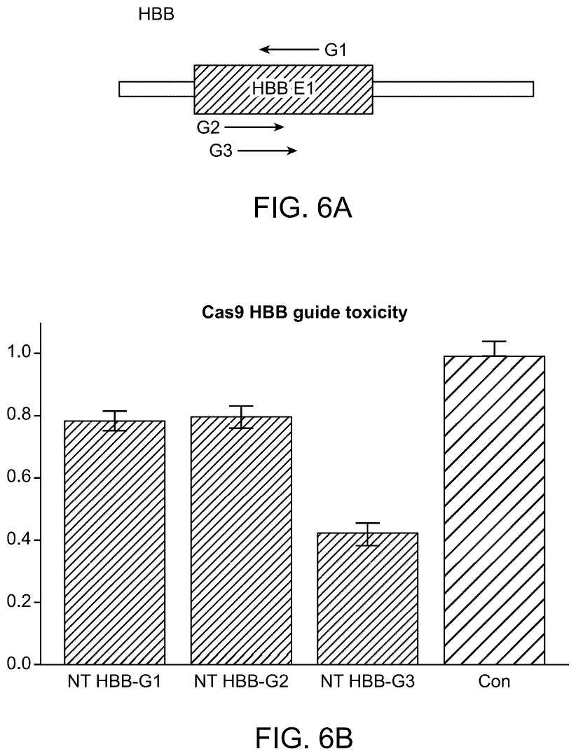

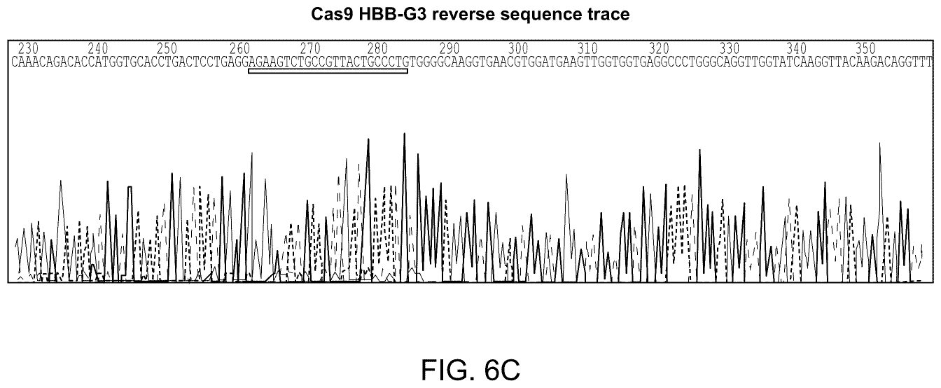

FIG. 6 shows a bar graph displaying normalized fold change of resorufin fluorescence of cells transfected with RNP plasmids with different gRNA sequences to control cells. Panel A of FIG. 6 shows three gRNA sequences (G1, G2, and G3) designed to target Exon 1 of the HBB gene. In Panel B of FIG. 6, the y-axis displays numbers of normalized fold change and the x-axis displays columns of NT HBB-G1, NT HBB-G2, NT HBB-G3, and Controls. The control's resorufin fluorescence measurement is normalized to 1, accordingly, their normalized fold change number is 1. The NT HBB-G3 has the smallest normalized fold change number, which indicates it has the least resorufin fluorescence. Panel C of FIG. 6 shows Cas9 HBB-G3 reverse sequence trace indicating generation of INDELs, linking toxicity to nuclease cleavage activity.

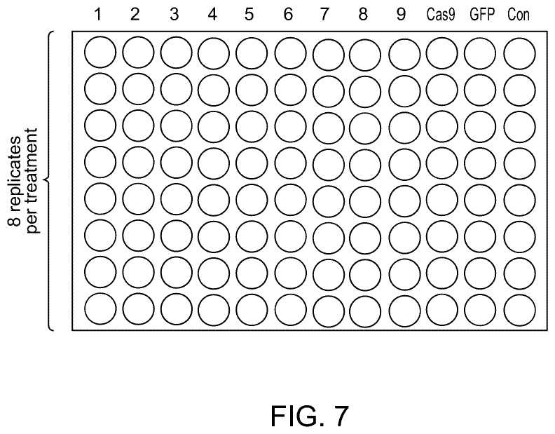

FIG. 7 shows an embodiment of experiment design. Cells are cultured in a 96-well plate and each well is seeded with about 2.5×10⁴ cells. Each column of the 96-well plate receives a treatment with different plasmid respectively. Because each column contains 8 wells, each treatment has 8 replicates. The cells in the first column are transfected with plasmids encoding the first fusion protein as shown in FIG. 1; the cells in the second column with plasmids encoding the second fusion protein as shown in FIG. 1; the cells in the third column with plasmids encoding the third fusion protein as shown in FIG. 1; the cells in the fourth column with plasmids encoding the fourth fusion protein as shown in FIG. 1; the cells in the fifth column with plasmids encoding the fifth fusion protein as shown in FIG. 1; the cells in the sixth column with plasmids encoding the sixth fusion protein as shown in FIG. 1; the cells in the seventh column with plasmids encoding the seventh fusion protein as shown in FIG. 1; the cells in the eighth column with plasmids encoding the eighth fusion protein as shown in FIG. 1; and the cells in the ninth column with plasmids encoding the ninth fusion protein as shown in FIG. 1. Further, the cells in the tenth column are transfected with plasmids encoding unmodified Cas9 enzymes, while the cells in the eleventh column are transfected with plasmids encoding unmodified Cas9 enzymes as well as plasmids encoding hExo1(1-352). The cells in the twelfth column are transfected with plasmids encoding GFPs. The cells in the thirteenth column are controls without any treatment.

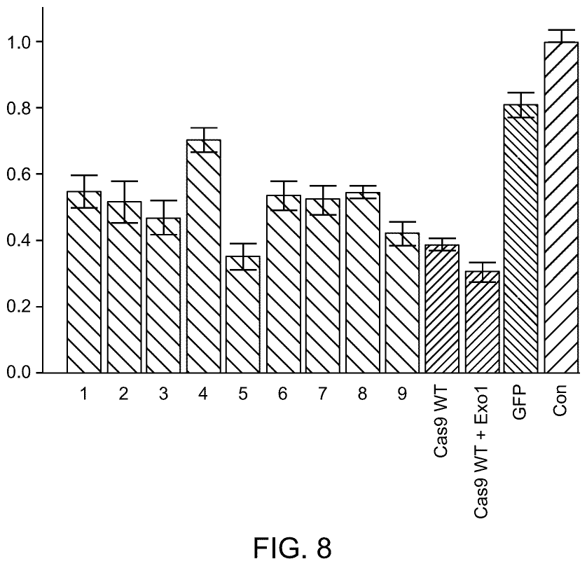

FIG. 8 shows a bar graph displaying normalized fold change of the measured resorufin fluorescence. The y-axis displays numbers of normalized fold change and the x-axis displays treatments with RNP plasmids encoding the 9 fusion proteins respectively and G3 (SEQ ID NO: 23) gRNA. The x-axis further displays two additional treatments: one transfecting the cells with RNP plasmids encoding unmodified Cas9 and G3 gRNA (Cas9 WT); and the other one transfecting the cells with RNA plasmids encoding unmodified Cas9 and hExo1separately and G3 gRNA (Cas9 WT +Exo1). Moreover, the x-axis displays GFP treatment group and Control group without any treatment. The Control group's resorufin fluorescence measurement is normalized to 1 as the normalized fold change number is 1. Every other treatment's resorufin fluorescence measurement is compared to the Control group's to obtain the normalized fold change number. As expected, different degrees of cellular toxicity are observed from all the other treatments, with the Cas9 and Cas9+hExo1 groups showing the smallest normalized fold change numbers, which indicates that the transfected cells from the two positive control groups have the least amount of resorufin fluorescence, and additionally demonstrating the necessity for direct fusion of hExo1to Cas9 for toxicity reduction.

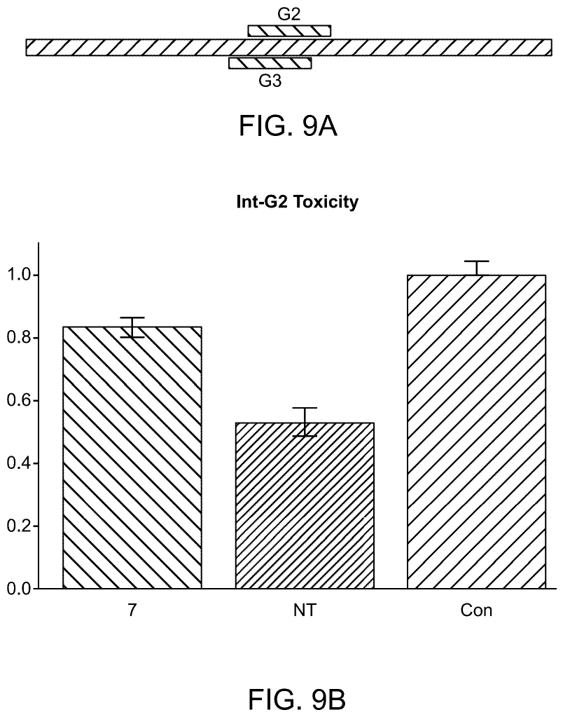

FIGS. 9A-B show a bar graph displaying normalized fold change of the measured resorufin fluorescence. FIG. 9A shows the G2 and G3 gRNA targeting the exon 1 of the HBB gene. In panel FIG. 9B, the y-axis displays numbers of normalized fold change and the x-axis displays treatments with RNP plasmids encoding the seventh fusion protein with G3 gRNA and RNP plasmids encoding an unmodified Cas9 and G2 gRNA and Control. The Control group's resorufin fluorescence measurement is normalized to 1 as the normalized fold change number is 1. Every other treatment's resorufin fluorescence measurement is compared to the Control groups to obtain the normalized fold change number. The RNP plasmids encoding an unmodified Cas9 and G2 gRNA group displays the lowest normalized fold change number, which indicates the transfected cells from this group have the least amount of resorufin fluorescence.

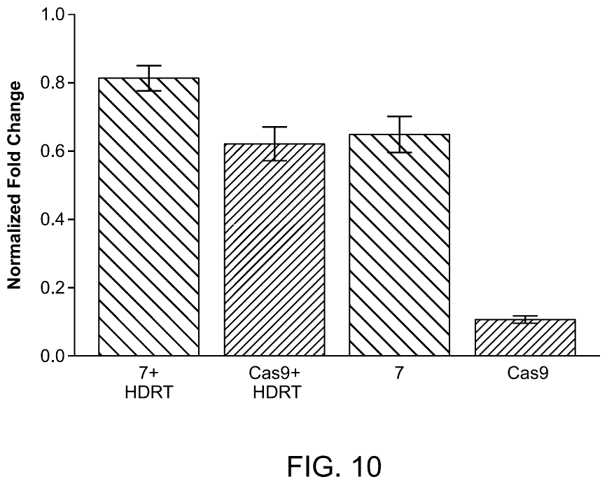

FIG. 10 shows a normalized fold change of resorufin fluorescence of cells transfected with different RNP plasmids targeting exon 1 of HBB gene with or without a single-stranded Homology Directing Repair Template (HDRT). Both Cas9-HR7 with and without HDRT shows increased resorufin fluorescence (hence decreased cellular toxicity). Additionally, addition of HDRT reduces toxicity in both Cas9-HR7 and wild-type Cas9 (NT), however we are unsure whether this affect is specific (requiring homology arms for HBB exon 1), or if the HDRT is simply competing for transfection with the plasmids encoding Cas9-HR7 and Cas9(NT). Regardless, Cas9-HR7 shows reduced toxicity.

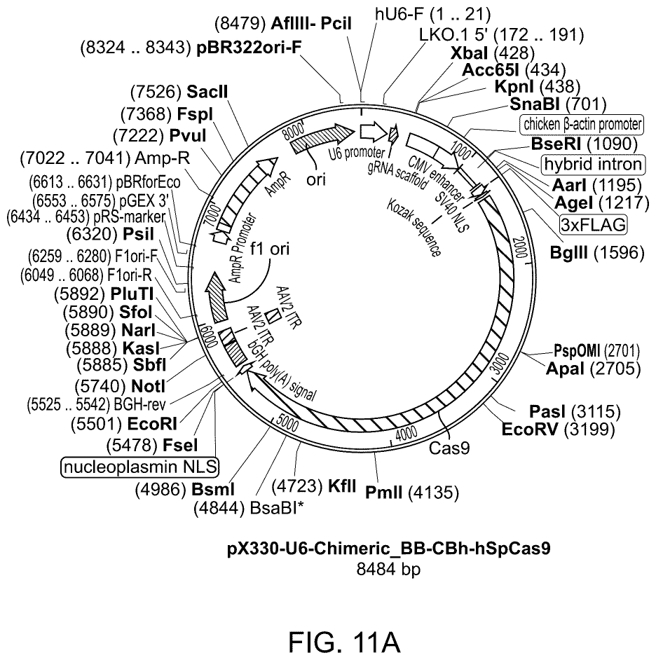

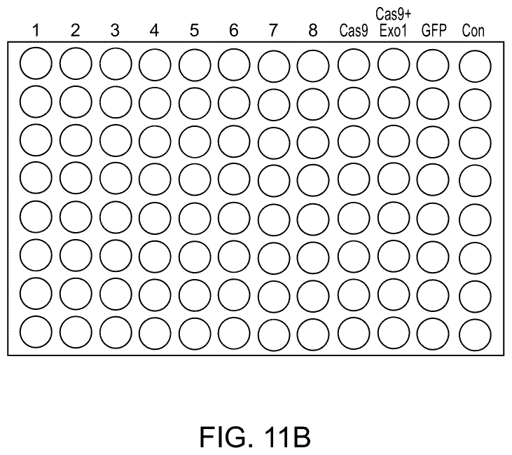

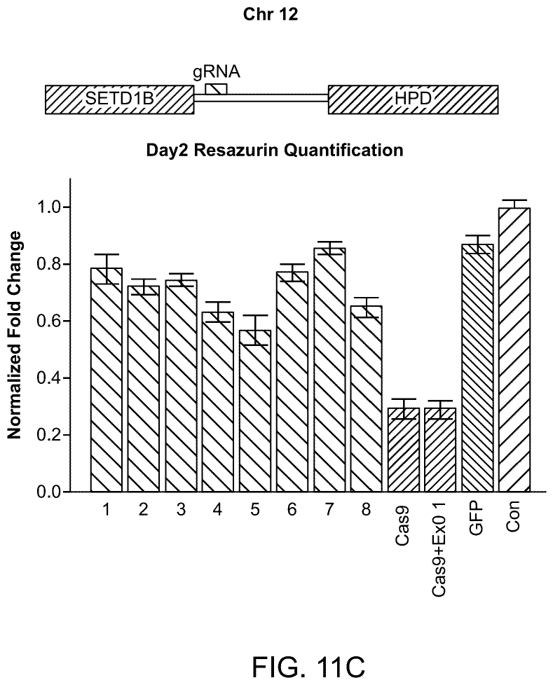

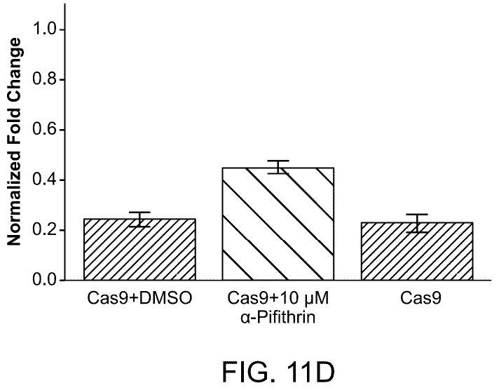

FIG. 11A is a diagram of Plasmid PX330 which contains a constitutive promoter for mammalian Cas9 expression, along with U6 promoter driven gRNA expression. This plasmid was modified to produce the various Cas9-HR versions 1-9.

FIG. 11B is an example of the experimental set up wherein cells are seeded in a 96 well glass bottom well plate. Cellular toxicity is quantified two days post-transfection via conversion of resazurin to resorufin, which is then normalized to a non-transfected control to allow for accurate comparisons across experiments. As indicated above the 96 well plate, each column is a different treatment, with 8 rows providing 8 independent replicates for each treatment.

FIG. 11C is a graph showing reduced cellular toxicity in A549 cells with gRNA targeting intergenic region on Chromosome 12. Cas9-HR constructs 1-8 have significantly less toxicity in A549 cells than unmodified Cas9, as shown by the higher normalized fold change values. The averages of Cas9-HR 1-8, Cas9, Cas9+hExo1, GFP and untransfected control (Con) are normalized to the Con average fluorescence. Importantly, physical coupling of Cas9 and hExo1 is necessary for toxicity reduction, as transfection of both Cas9 and hExo1 does not reduce toxicity relative to Cas9 alone. All experiments are done in duplicate independent well plates (16 replicates total), error bars represent the standard error of the mean.

FIG. 11D is a graph showing that treatment with alpha-pifithrin (10 micromolar) reduces Cas9 induced cellular toxicity in A549 cells. As an extension of FIG. 5, here it can be seen the addition of DMSO does not change toxicity relative to transfection of Cas9 alone, indicating that the effects seen with PFT-α are specific.

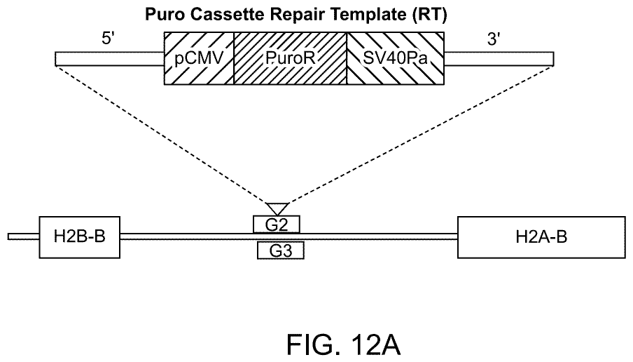

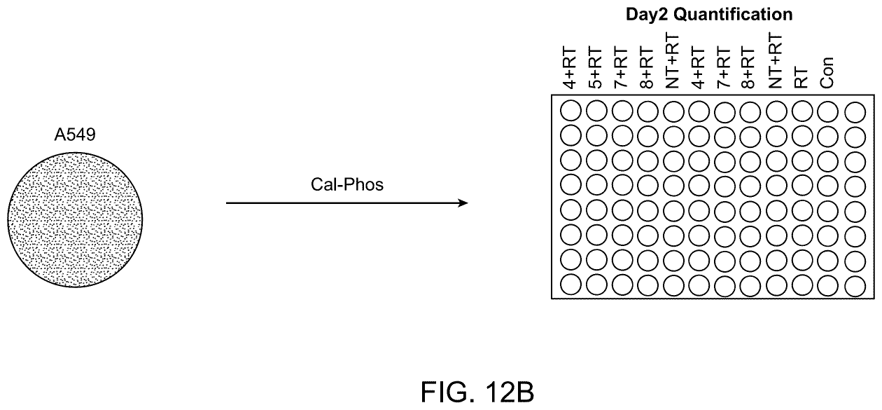

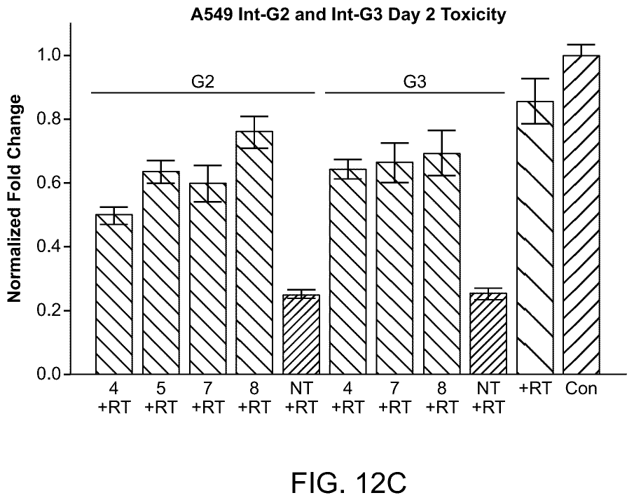

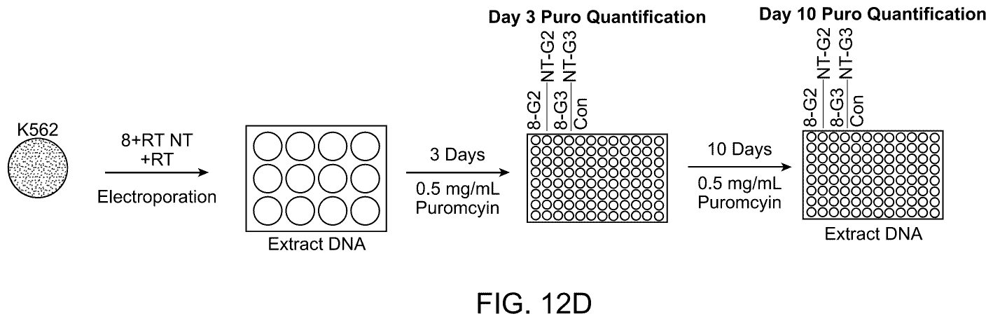

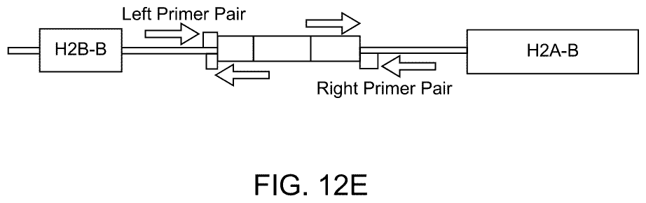

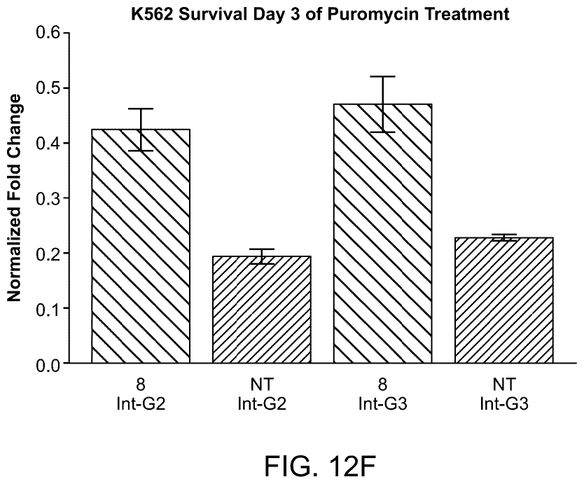

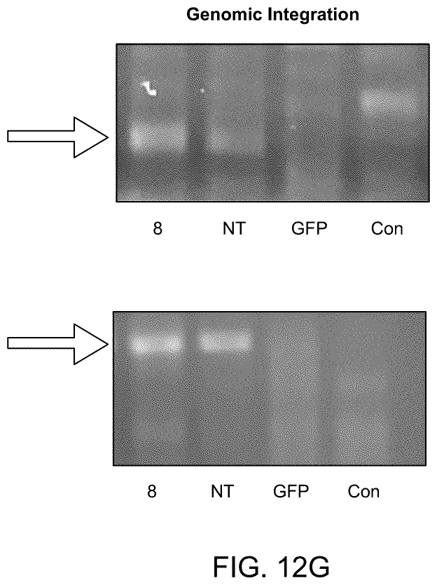

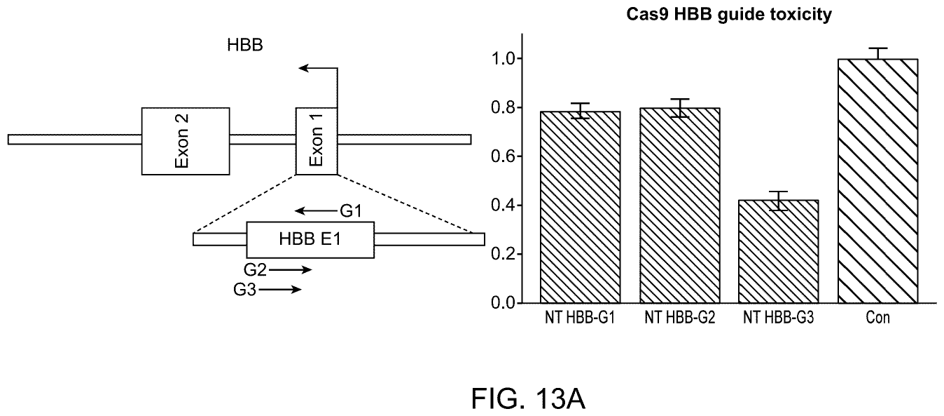

FIG. 12A is a diagram of a Puromycin resistance repair template (RT). It contains a 5′ homology arm (5′), a strong constitutive viral promotor (pCMV), a Puromycin Resistance gene (Puro), a poly-A sequence (SC40Pa), and a 3′ Homology Arm (3′). Below the repair template is the genomic region targeted by guides Int-G2 and G3. The repair template is designed to integrate in the middle of both guide sequences, thereby preventing further Cas9 cleavage. The integration site is in an intergenic region between H2B-B and H2B-A on Chromosome 6, which allows testing of the ability of Cas9-HR to function in both intergenic and coding regions of the genome. Furthermore, both strands are targeting, testing Cas9-HRs compatibility with both sense and anti-sense orientation.

FIG. 12B shows the method used to quantify toxicity of hExo-Cas9 fusions in A549 cells. A549 cells were plated in 96 well plates, 500 ng of each plasmid and 100 ng of repair template were transfected via a standard Cal-Phos protocol, as described previously.

FIG. 12C is a graph depicting the toxicity of various constructs tested via a resazurin assay. All Cas9-HR constructs show significantly less toxicity (higher normalized fluorescence) than Cas9-NT, with 8 having no statically significant difference in toxicity than repair template only controls. Additionally, Cas9-HRs targeting both sense and anti-sense showed similar reductions in toxicity, indicating Cas9-HR can function in either orientation.

FIG. 12D depicts the method of the assay to measure HDR activity of Cas9-HR8 and Cas9. This assay identifies the rate of HDR via measuring cellular survival to treatment with Puromycin. Because this is a survival assay, and A549 cells showed significant p53 dependent cellular toxicity to transfection of Cas9, K562 cells (p53 −/−) were used instead in order to facilitate accurate quantification of HDR rate. K562 cells were aliquoted in 12 well plates after electroporation with 500 ng of either Cas9-HR8 or Cas9 and 100 ng of repair template. After two days DNA was extracted and Puromycin (0.5 mg/mL) selection was initiated. After three days of selection, K562 cells were quantified via resazurin in 96 well plates.

FIG. 12E is a depiction of the genomic regions of cells successfully integrated by the Puro-RT. The left primer pair and right primer pair are designed so that one primer binds in the genomic region outside of the repair template, while the other binds a sequence specific to the puromycin cassette. Successful amplification of both 5′ and 3′ primer sets strongly indicates correct integration of the transgene.

FIG. 12F shows the survival data of K562 cells transfected with either Cas9-HR8 or Cas9 with G2 or G3 gRNA after three days of puromycin treatment. Data was normalized to cells transfected with a plasmid containing the RT. Cas9-HR8 targeting Sense (Int-G2) and anti-sense (Int-G3) showed greater than a two-fold increase in normalized resorufin fluorescence relative to wildtype Cas9 (NT) after 3 days of puromycin selection. A two-fold increase in resorufin fluorescence translates to at least a two-fold increase in HDR rate, showing that not only can Cas9-HRs can dramatically reduce toxicity, they also can increase HDR rate and Cas9-HR functions in multiple cell types.

FIG. 12G shows an agarose gel after amplification of the target region by both the 5′ and 3′ primer pairs showing that the repair template had been successfully integrated by 8 th fusion protein of FIG. 1 and Cas-9 control (NT). There was no amplification of the target region when transfected with only GFP construct (GFP) or without the template (Con).

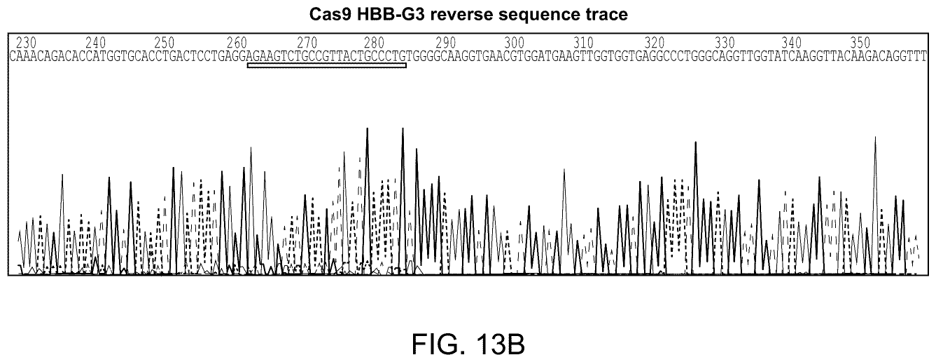

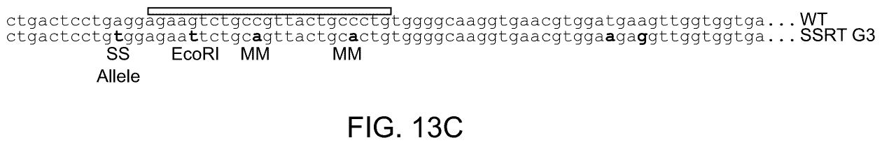

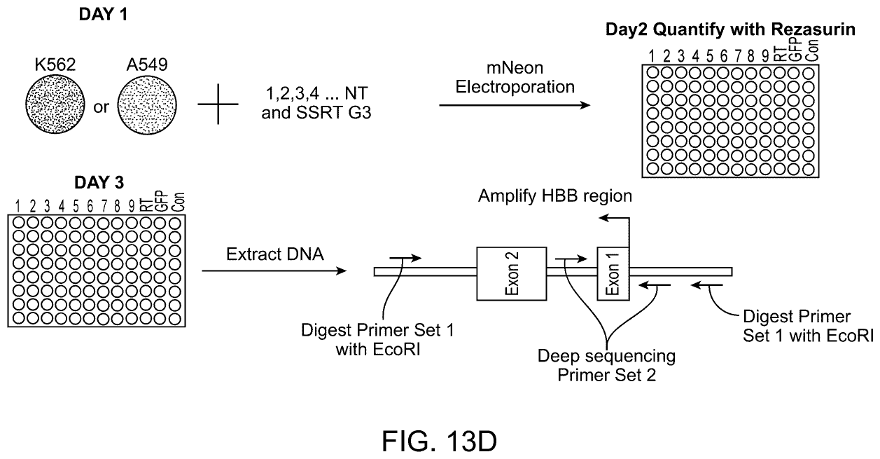

FIG. 13A shows the genomic region, including the first two exons of HBB targeted to edit the Human Hemoglobin Beta (HBB) gene. The inset shows a larger version of Exon 1, with a diagram depicting the gRNAs tested. The graph shows data from the toxicity screen of HBB gRNA guides in A549 cells. Toxicity experiments were performed as in FIG. 11D, with HBB-G3 showing higher toxicity than either HBB-G1 or HBB-G2.

FIG. 13B shows sanger sequencing of the HBB genomic region in the HBB-G3 treated A549 cells. Sanger sequencing shows characteristic noise following Cas9 cleavage and repair via NHEJ pathways, with the bar indicating the gRNA sequence. In this case the noise is 5′ as opposed to 3′ due to sequence with the reverse primer. Clear cleavage and repair via NHEJ could not be detected in cells treated with HBB-G1 or HBB-G2.

FIG. 13C is a diagram of the wild-type HBB sequence and the SSRT-G3 sequence which introduces the sickle cell (E6V), an EcoRI site which creates a mis-sense mutation, and four silent mismatch mutations (bolded a, a, a, and g nucleotide bases) with the HBB-G3 gRNA highlighted by the bar above. Single strand repair template (SSRT)-G3 is 120 bp long, with 60 bp arms on either side of the predicted cut site. Mutations are designed to prevent gRNA binding upon successful repair

FIG. 13D depicts a HBB editing experiment in which K562 cells or A549 cells are electroporated with Cas9+SSRT-G3, Cas9-HR 1-9+SSRT-G3 or SSRT-G3 alone. After two days the cells are quantified as in FIG. 11D. DNA is then extracted and the HBB locus is amplified with two primer pairs. The outer pair is digested with EcoRI to quantify the HDR editing rate and the inner pair can be used for deep sequencing to provide an independent quantification of HDR rate in addition to INDEL rate, allowing for accurate quantification of the HDR/INDEL ratio.

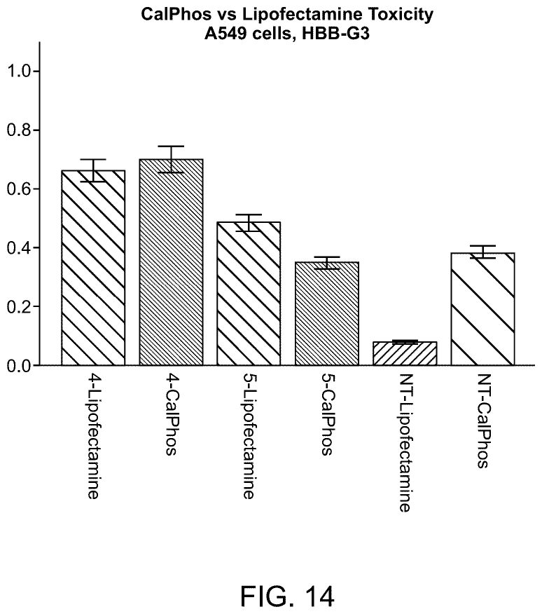

FIG. 14 illustrates toxicity assessment of two transfection methods, lipofectamine and calcium phosphate (CalPhos) as determined by transfecting A549 cells with HBB-G3 gRNA and Cas9-HR fusion proteins 4 and 5 as depicted in FIG. 1. The similar results using either Cal-Phos or lipofectamine seen Cas9-HR4 and 5 strongly indicate that the toxicity effects are not dependent on particular transfection reagents/methods. Additionally, lipofectamine transfection of Cas9(NT) showed increased toxicity relative to Cal-Phos transfection, indicating that Cal-Phos transfection may actually underreport the reduction of toxicity by Cas9-HRs compared to likely more efficient lipofectamine transfection.

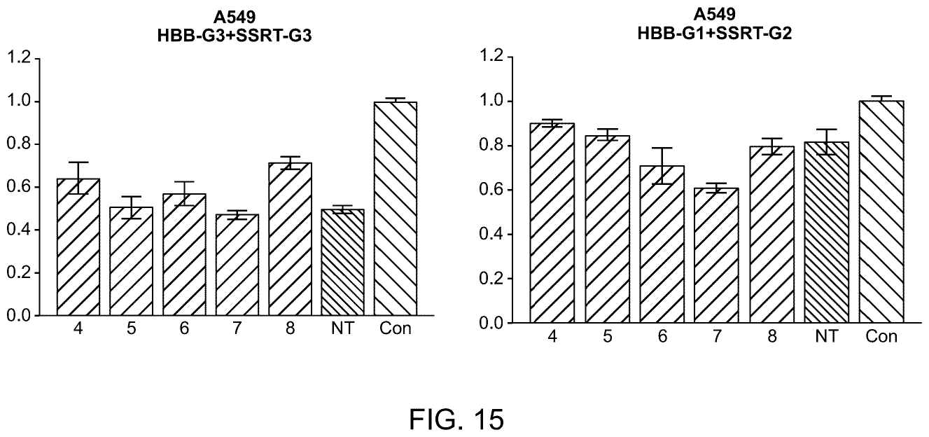

FIG. 15 illustrates toxicity assessment by transfecting A549 cells with SSRT HBB repair templates of FIG. 13A. Resazurin levels are measured on day 2 after the transfection. Cas9-HR fusion proteins 4 and 8 are less toxic in A549 cells. SSRTs reduces toxicity cellular toxicity, particularly for NT.

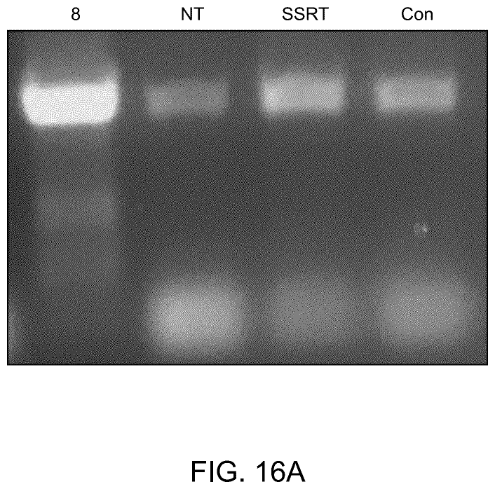

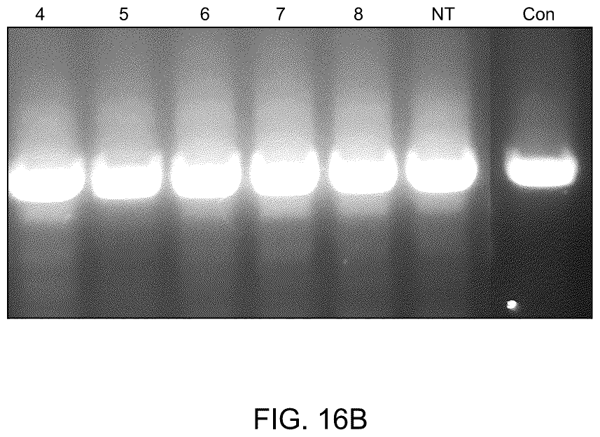

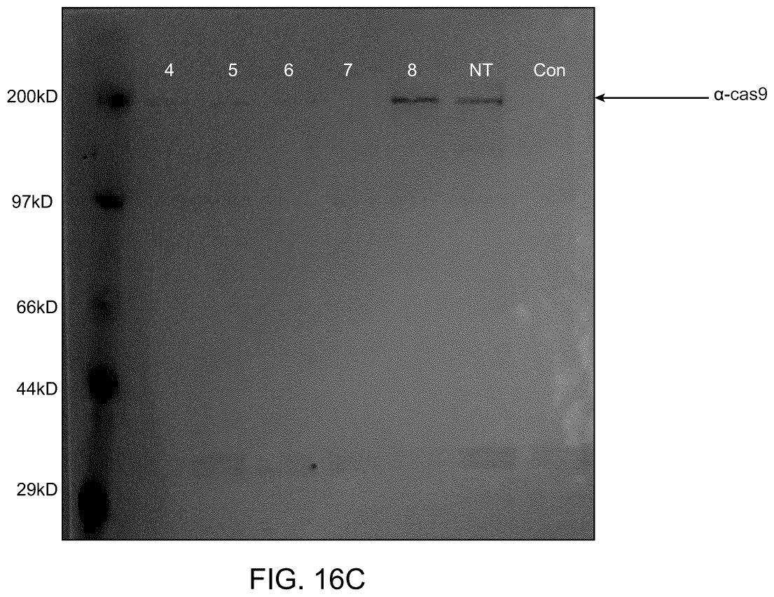

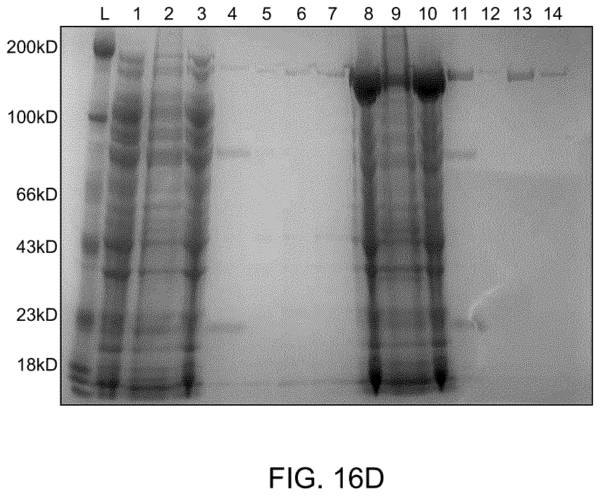

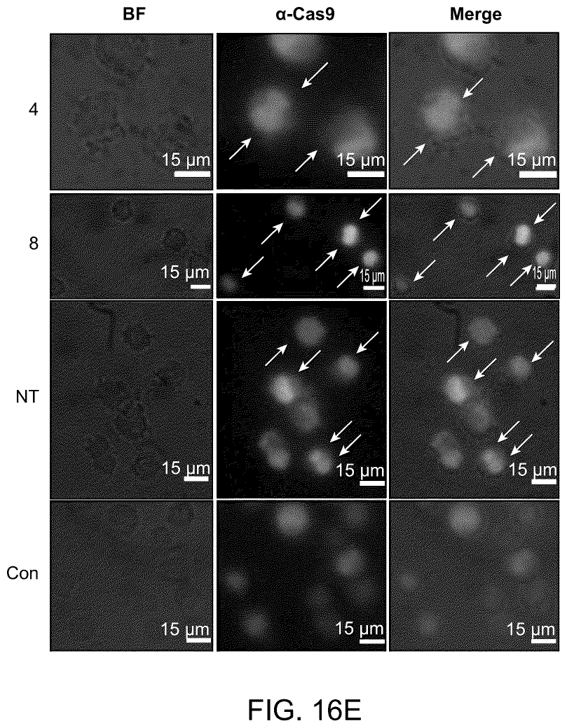

FIG. 16A shows an agarose gel of EcoRI digestion assay depicting Cas9-HR fusion protein 8 of FIG. 1 integrating the HBB repair template into the genome of K562 cells. Arrows depict the EcoRI digested products. There are no detectable EcoRI digested products in lanes of Cas9 only (NT), SSRT, and Con (no Cas9). This shows that Cas9-HRs are flexible in repair template choice, and both SSRTs and double strand (DS)RTs can be used for genomic edits.

FIG. 16B shows an additional agarose gel of EcoRI digestion assay depicting Cas9-HR fusion proteins 4, 5, 6, 7, and 8 of FIG. 1 integrating the HBB repair template into the genome of K562 cells. Arrows depict the EcoRI digested products, indicating successful HDR. As expected given previous toxicity results (FIG. 8), Cas9-HR4 appears to have the highest HDR rate, with all other Cas9-HRs and Cas9(NT) showing some level of successful HDR when compared to digestion of an untransfected control (Con).

FIG. 16C shows a western blotting of Cas9-HR fusion proteins 4, 5, 6, 7, and 8 (as shown in FIG. 1), Cas9 only (NT), and Con (no Cas9). Arrow indicates detection of Cas9 in Cas9-HR fusion proteins and NT lanes. While amounts appear lower for fusions 4-7, additional blots and IHC (FIG. 16E) show that proper expression and localization of all Cas9-HRs, indicating that the reduction of toxicity is not likely due to a reduction in expression levels. As an example, Cas9-HR4 and 8 are some of the lowest and highest expressors of the Cas9-HRs assayed by western blot. If cellular toxicity was truly reduced by reducing expression, it would be expected that Cas9-HR 4 would have the lowest toxicity at every target tested in the genome. However, that is not the case, as FIG. 11C and FIG. 12C show that Cas9-HR4 actually has the highest toxicity of all the Cas9-HRs tested, whereas Cas9-HR8 is among the least toxic. Given these results, it is much more likely that expression levels play no significant role in determining cellular toxicity, further evidenced by the fact that Cas9-HR4 has the greatest reduction of toxicity of all Cas9-HRs tested when targeting HBB exol (FIG. 8), thus showing no clear correlation between expression level and toxicity. Finally, it is interesting to note that Cas9-HR 4 and 8 appear to show complimentary reductions in toxicity at various sites in genome: if Cas9-HR4 reduces toxicity, Cas9-HR8 does not (or less effectively does so), and vice-versa. This may speak to the different local chromatin environments in these different environments, with the possibility that the different linker identities allow for optimal positioning of the hExo1domain in different chromatin environments. Therefore, the use of different versions of Cas9-HR may allow for reduction of toxicity (and increase in HDR) at virtually all locations throughout the genome.

FIG. 16D illustrates successful expression and purification of Cas9-HR3 from E. Coli monitored via SDS-PAGE with Coomassie staining. Lane L is ladder. Lanes 1 and 8 are soluble fractions of cell lysate. Lanes 2 and 9 are insoluble lysed cell pellet. Lanes 3 and 10 are flow-throughs of the soluble fractions passing through a Nickle (Ni-NTA) column. Lanes 4 and 11 are elution fractions where proteins bound to the Nickle are eluted. Lanes 5 and 12 are follow-throughs of sulphoproyl (SP) cation exchange chromatography resin. Lanes 6 and 13 are elution fractions eluted with 500 mM NaCl. Lanes 7 and 14 are elution fractions eluted with 1M NaCl. Lanes 1-7 are from cells transfected with Cas9-HR3. Lanes 8-14 serve as controls for purification protocol and are from E. coli expressing only unmodified Cas9. Development of successful E. coli based protein purification protocol for Cas9-HRs allows for both in-vitro tests of Cas9-HR activity, as well as direct RNP transfection and editing of various eukaryotic organisms.

FIG. 16E illustrates immunohistochemistry (IHC) of same transfected cells from FIG. 16C. Arrows indicate that Cas9-HR fusions and Cas9 are localized to the nucleus of the cells. Both detection and proper localization of all Cas9-HR4-8 (5-7 assayed and proper localization seen, data not shown) in the nucleus further demonstrate that the reduction of toxicity by Cas9-HRs is not due to improper localization nor significant reduction in expression levels as assayed by IHC.

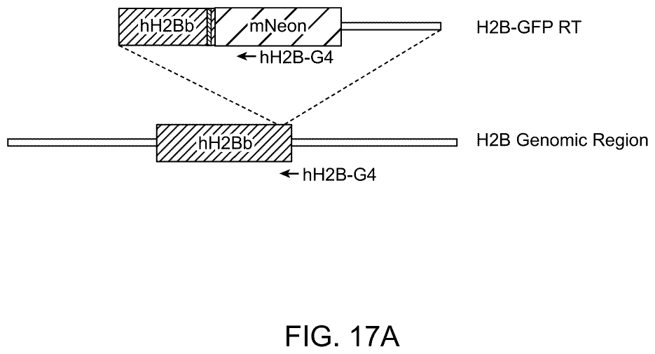

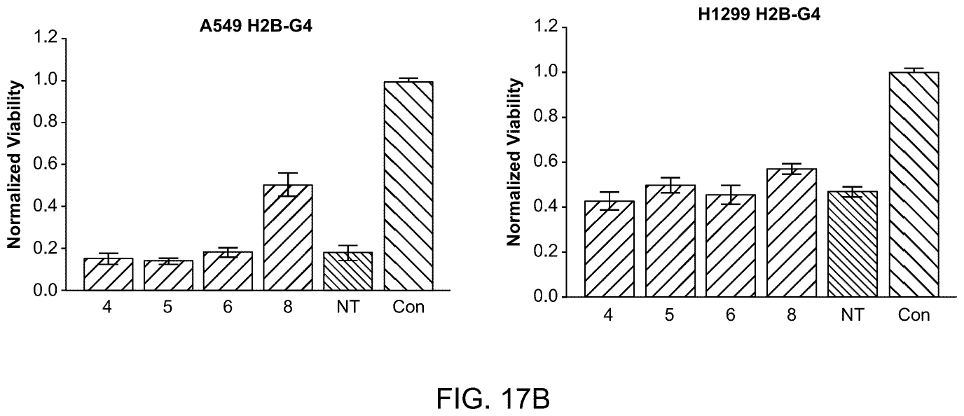

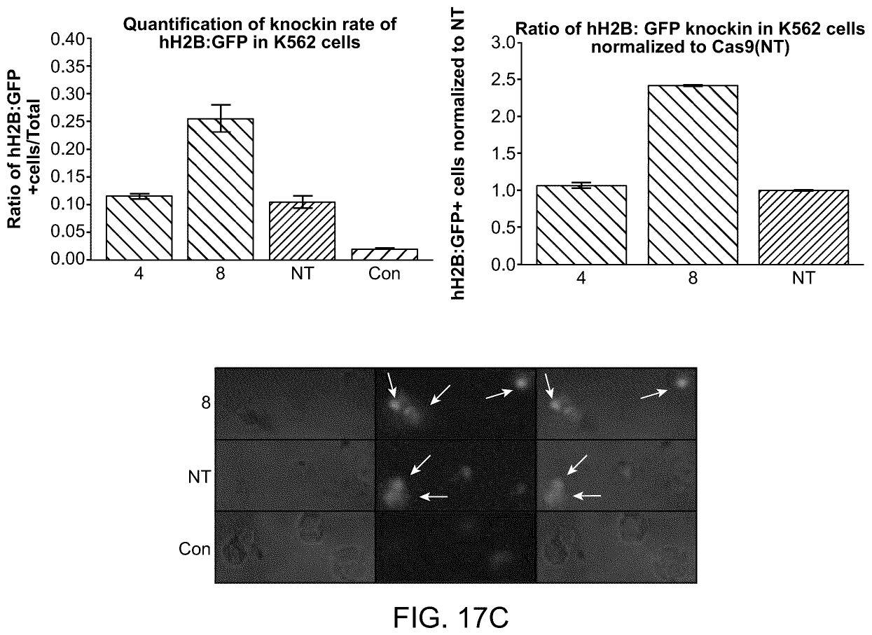

FIG. 17A illustrates the design of the repair template for an H2BmNeon knock-in experiment. This experiment allows for accurate quantification of HDR rate via properly localized GFP fluorescence in a non-survival based assay.

FIG. 17B illustrates p53-depedent decrease of cellular toxicity induced by Cas-HR fusion proteins 4, 5, 6, and 8 of FIG. 1, Cas9 only (NT), and Con (no Cas9) in epithelial lung cancer cell lines. A549 cells are positive for p53 activity, while H1299 cells are negative for p53 activity. Toxicity as determined by normalized resazurin levels (y-axis) has shown that absence of p53 in H1299 cells yields lower cellular toxicity. In A549 cells, only Cas9-HR8 shows a significant decrease in toxicity relative to Cas9(NT) , while Cas9-HR4-7 are similar to NT. However, in H1299 cells, toxicity decreases dramatically for Cas9-HR4-7 and NT to roughly the level seen in A549 with Cas9-HR8, while Cas9-HR8 slightly decreases toxicity even further. As with previous experiments it is anticipated that the different orientation of the hExo1domain due to different linker identity influences the likelihood of end resection, and therefore commitment to HDR. In this case, Cas9-HR8 has the highest rates of HDR, which is directly tested in FIG. 17C. This also further corroborates the results seen in A549 cells with PFT-α, as it is likely that the loss of p53 function in H1299 vs A549 cells drives the significant reduction in toxicity seen in Cas9-HR4-7 and NT. Additionally, it is noted that Cas9 HR8 has reduced toxicity relative to the other fusion proteins in H1299 cells.

FIG. 17C illustrates the assessment of successful tagging of H2B (via GFP+ cell quantification) FIG. 17A in K562 cells. Arrows in IHC images indicate correct expression and localization of cells with successful H2BmNeon knock-in. The data from this experiment show again that reduction of toxicity in A549 cells is linked with increase in HDR rate in K562 cells, indicating that reduction of toxicity by Cas9-HRs in p53+ cells may serve as a proxy for HDR rate. Importantly, this is an non-survival based assay which also shows an at least two fold increase (2.5× for Cas9-HR8) in HDR rate compared to Cas9(NT). Additionally, this experiment shows that Cas9-HRs can function equally well in both intergenic (FIG. 12F) and coding sequences (this experiment).

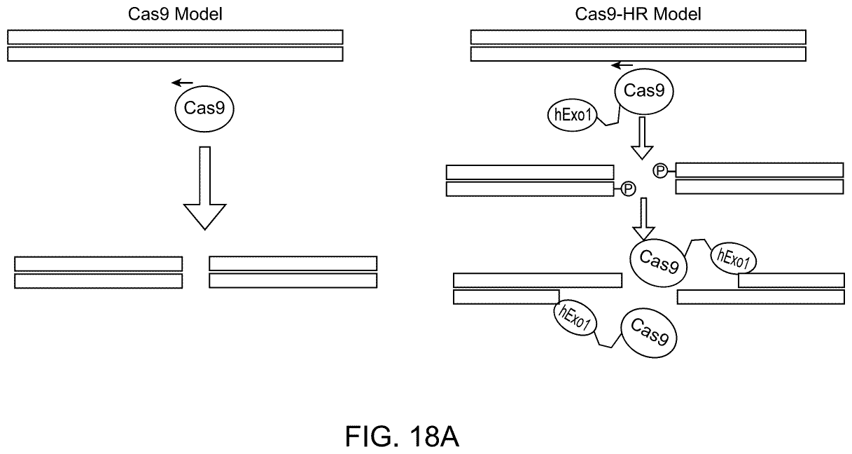

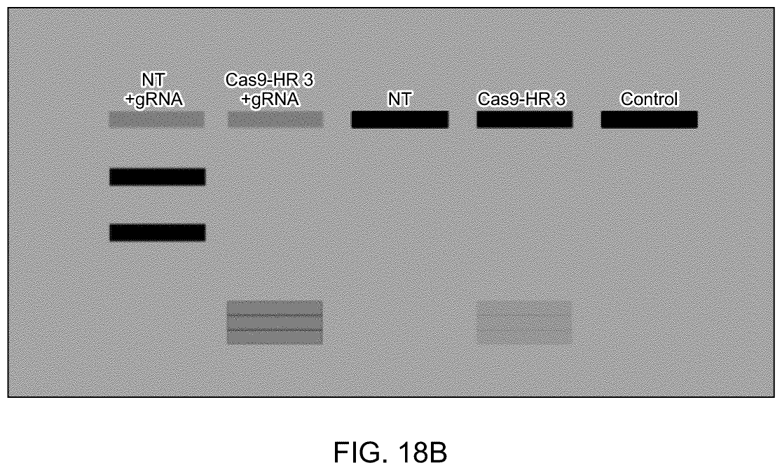

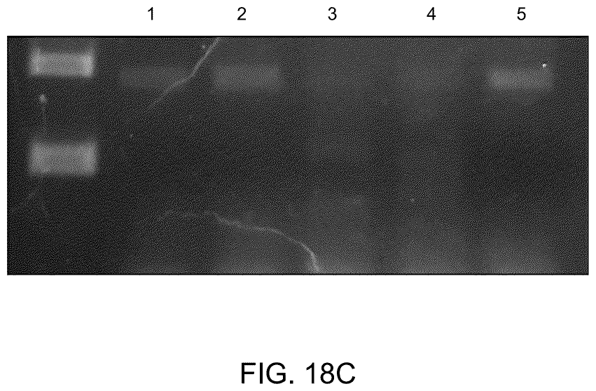

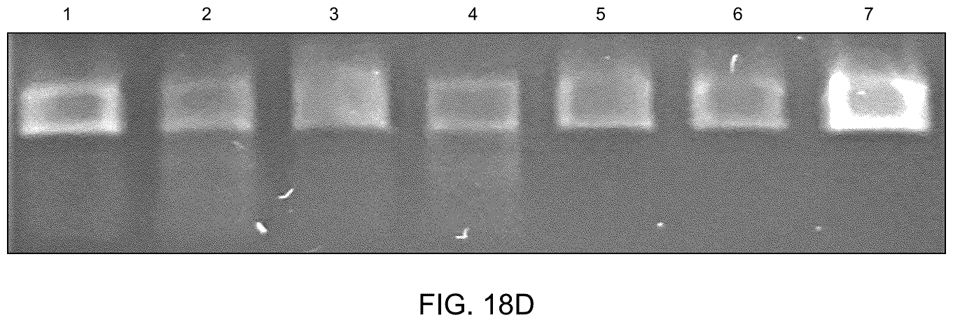

FIG. 18A illustrates the schematic difference between the experimentally verified Cas9 only model and the theoretical Cas9-HR model. The presence of an Exonuclease domain fundamentally changes the predicted in-vitro cleavage pattern. Exo1 has a significant preference for phosphorylated 5′ termini vs non-phosphorylated termini. Therefore, theoretically when using PCR products or other pieces of DNA normally lacking 5′-phosphorlyated termini that endonuclease cleavage via Cas9 can dominate initially, whereas after cleavage the two fragments will each possess 5′-phosphorlyated termini, which can result in rapid degradation via the hExo1 domain.

FIG. 18B illustrates an exemplary digestion pattern based on FIG. 18A. Only Cas9-HR3+gRNA and Cas9-HR3 can produce the digested products which demonstrate successful in-vitro nuclease activity. Additionally, though hExo1strongly prefers phosphorylated 5′-termini, hExo1can still bind and resect unphosphorylated 5′-termini, so a small amount degradation without gRNAs may be seen with the addition of Cas9-HRs without gRNA.

FIG. 18C illustrates an actual agarose example of FIG. 18A and FIG. 18B. Genomic DNA was amplified with primers amplifying a roughly 950 bp region surrounding HBB Exon 1. Lanes 1 and 2 show Cas9-HR3 with gRNAs HBB-G1 or HBB-G3, Lanes 3 and 4 show Cas9 (NT) with gRNAs HBB-G1 or HBB-G3, Lane 5 is an untreated control. Cas9 cleavage patterns are as expected based on the verified model, with both HBB-G1 and G3 showing strong cleavage, with a clear reduction of the initial product (950 bp) and accumulation of cleavage products (pairs of bands ˜550-300 bp). The cleavage pattern of Cas9-HR3 also matches the predicted pattern, with a clear reduction in the intensity of the large initial product (950 bp), demonstrating that Cas9-HR3 retains functional guided endonuclease activity. Additionally, compared to Cas9, Cas9-HR3 doesn't produce any intermediately sized cleavage products (650-300 bp), likely due to digestion via hExo1 domain. Therefore, these results show that Cas9-HR3 shows both expected enzymatic activities (endo- and exo-nuclease) in-vitro.

FIG. 18D illustrates a similar experiment as FIG. 18C, which differs by conducting the experiment after leaving enzymes for 2 weeks at 4° C. in order to compare protein stability. Lane 1 is digestion pattern from the combination of Cas9-HR3 and gRNA HBB-G1. Lane 2 is digestion pattern from the combination of Cas9 and gRNA HBB-G1. Lane 3 is digestion pattern from the combination of Cas9-HR3 and HBB-G3. Lane 4 is digestion pattern from the combination of Cas9 and HBB-G3. Lane 5 is digestion pattern from Cas9-HR only. Lane 6 is digestion pattern from Cas9 only. Lane 7 is the control where there is neither Cas9 nor gRNA. These results show that both Cas9-HR3 and Cas9 have similar levels of stability.

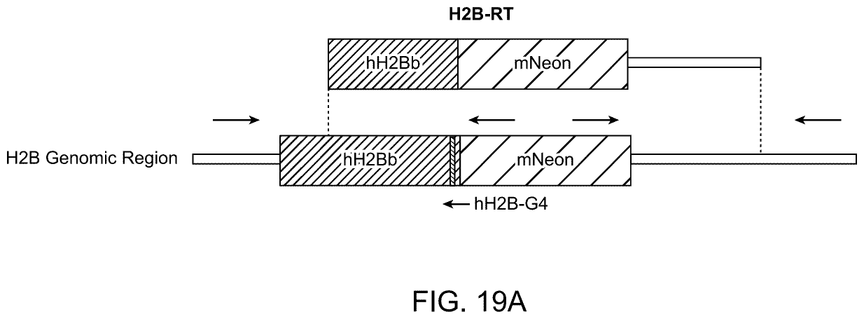

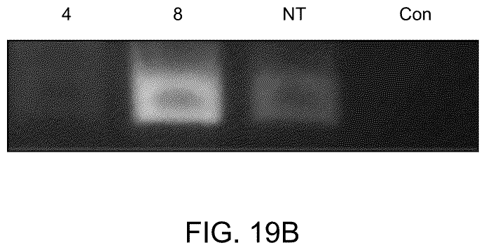

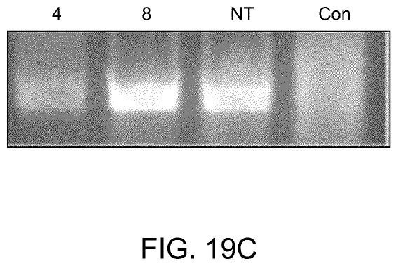

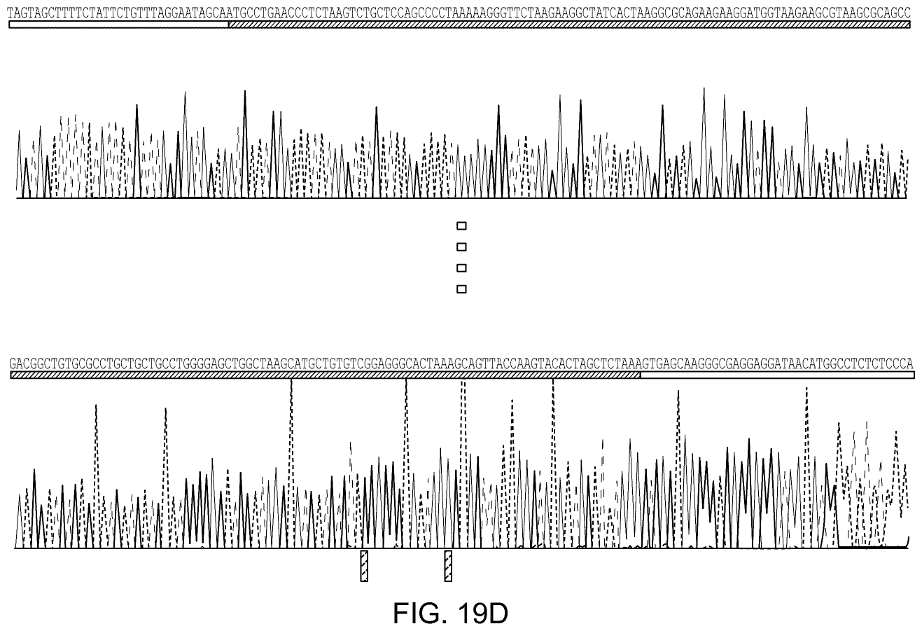

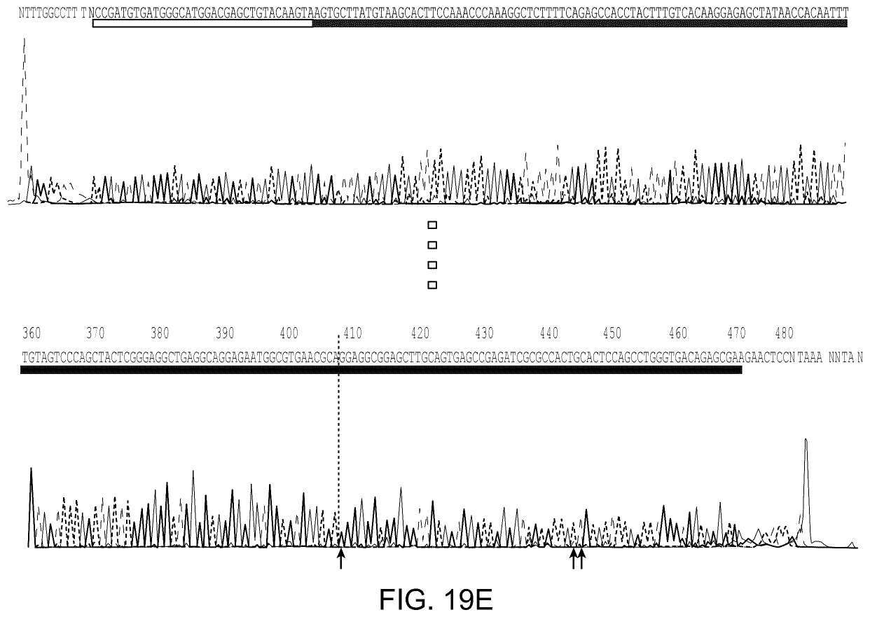

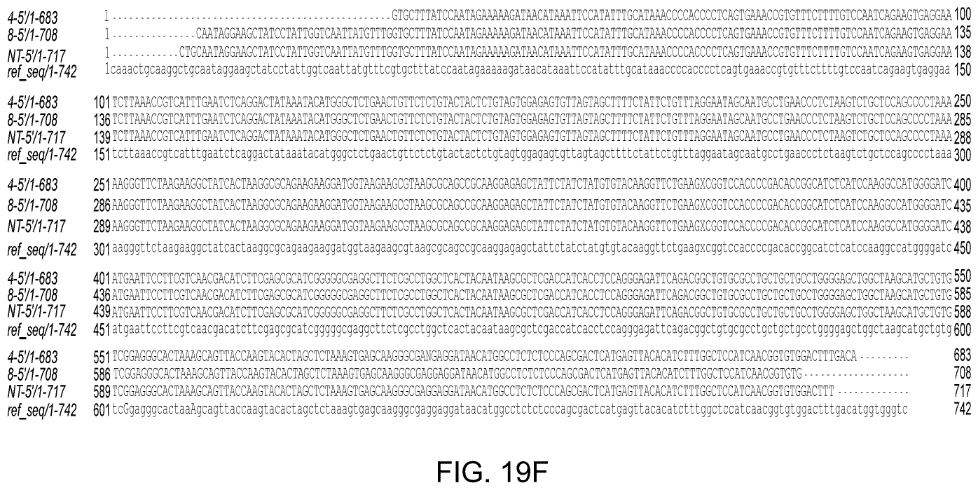

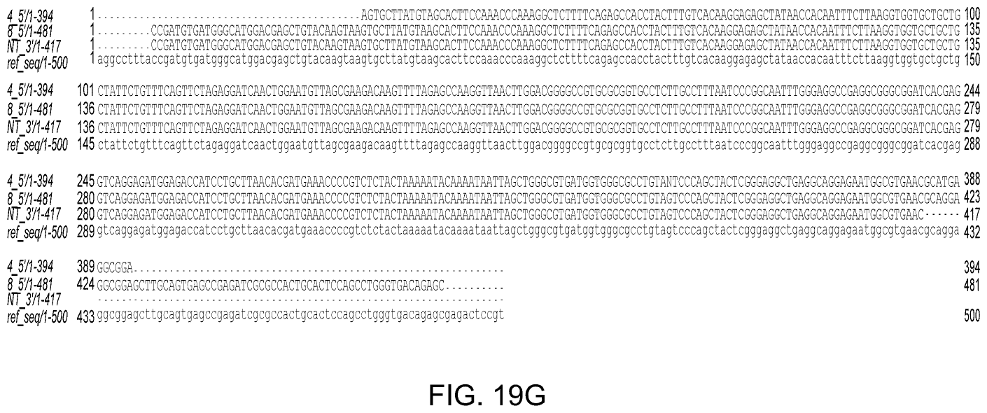

FIG. 19A illustrates design of H2B integration detection primers. Two sets of primers are designed to bind outside of the 5′ and 3′ ends of the repair template annealing to sequences only present in the genome, not in the RT, while the others anneal to sequences specific to the repair template, and are not present in the unmodified cells. Successful amplification of both the 5′ and 3′ set of primers strongly indicates successful and proper tagging of H2B with mNeon.

FIG. 19B illustrates an agarose gel showing PCR products amplified from gDNA extracted from K562 cells transfected with Cas9-HR4,8 and Cas9NT plus H2BmNeon RT along with an untransfected control (lanes 4,8,NT and Con). Amplification with the 5′ primers and gDNA from Cas9-HR 4, 8 and Cas9(NT) all show successful amplification of the 5′ product, while Con does not, indicating proper integration of the 5′ end of the RT. Additionally, the higher amount of amplified product using gDNA from Cas9-HR8 corresponds to the higher rates of HDR seen in FIG. 17C.

FIG. 19C PCR products amplified from gDNA extracted from K562 cells transfected with Cas9-HR4,8 and Cas9NT plus H2B-mNeon RT along with an untransfected control (lanes 4,8,NT and Con). While levels of Cas9-HR8 and Cas9 appear similar, given the significantly higher amplification of these two it is likely that the reaction had proceeded past the exponential phase, making quantification less reliable. Regardless, amplification with the 3′ primers and gDNA from Cas9-HR 4, 8 and Cas9(NT) all show successful amplification of the 3′ product, while Con lane shows no specific bands, indicating proper integration of the 3′ end of the RT.

FIG. 19D illustrates absorbance of sequence trace from Sanger sequencing of the PCR product amplified by the 5′ primers from Cas9-HR8. The top trace shows the 5′ sequence of the product, with the white bar showing sequences only present in the genome, while the shaded bar shows sequences present in both the RT and genome. The intervening sequences are cropped out, and the bottom trace shows the 3′ end of the product. The shaded bar again represents the H2B ORF, while the white bar represents mNeon. Additionally, the shaded bars show the two silent mutations introduced to prevent additional cleavage after transgene integration. Both Cas9-HR4 and Cas9(NT) traces were the same.

FIG. 19E illustrates absorbance of sequence trace from Sanger sequencing of the PCR product amplified by the 3′ primers from Cas9-HR8. The top trace shows the 5′ sequence of the product, with the white bar showing mNeon, while the shaded bar shows sequences present in both the RT and genome. The intervening sequences are cropped out, and the bottom trace shows the 3′ end of the product. The shaded bar again represents the H2B 3′ region, with the dashed line showing the transition from genome and RT to only genomic sequences. Additionally, three arrows show SNPs relative to the reference sequences. Cas9-HR4 contained similar mutations, whereas the Cas9 trace became degraded right after the end of the RT. It is much more likely that these represent bonified SNPs, though it cannot be ruled out that Cas9-HRs may induce some errors around the junction site. Direct sequencing of the control cells would help to resolve this.

FIG. 19F illustrates sequencing alignment of the PCR product amplified by the 5′ primers. No errors are seen relative to the expected reference sequence.

FIG. 19G illustrates sequencing alignment of the PCR product amplified by the 3′ primers. The only changes relative to the expected sequence are seen outside of the RT sequence, and most likely show cell line specific SNPs relative to the reference sequence.

FIG. 20 illustrates additional Cas9-HR fusion proteins with combinations of domains linked by at least two linkers to Cas9. These various fusions could possibly increase HDR rate and/or further decrease cellular toxicity.

### EXAMPLES

The following examples are given for the purpose of illustrating various embodiments as described in the present disclosure and are not meant to be limiting in any fashion. The present examples, along with the methods described herein are presently representative of preferred embodiments, are exemplary, and are not intended as limitations on the scope of the present disclosure. Changes therein and other uses which are encompassed within the spirit of the present disclosure as defined by the scope of the claims will occur to those skilled in the art.

### Example 1—Reduced Cellular Toxicity in A549 Cells

Referring to FIG. 1, several plasmid constructs with different polynucleotides were generated. Each polynucleotide encodes a different fusion protein comprised of a hExo1 fragment (Amino acids 1-352 of SEQ ID NO: 1) linked to a Cas9 (any of SEQ ID NO: 2-18) via a specific linker peptide. Some plasmid constructs encoded Cas9 enzymes with one N-terminal nucleus localizing sequence (NLS) or a C-terminal NLS and some plasmid constructs encoded Cas9 enzymes with both the C-terminal NLS and the N-terminal NLS. All plasmid constructs were sequenced to ensure that no mutations occurred in the polynucleotide sequences. Each of the plasmid constructs also contained a nucleotide sequence encoding a gRNA directed to an intended chromosomal site. The intended chromosomal site is in the intergenic region between VSP33A on the 5′ and CLIP1 on the 3′ on Chromosome 12 This region has no predicted genes or long non-coding RNA. Once the cells were transfected with the plasmids, Cas9-gRNA ribonucleoproteins (RNPs) were formed inside the cells. Control plasmids were prepared to encode unmodified Cas9 (any of SEQ ID NO: 2-18) enzyme.

Human lung carcinoma A549 cells were cultured and about 2.5×10⁴ cells were plated in 96-well plates, with 8-16 transfection replicates per individual treatment. Each well was then transfected with 62.5 ng of plasmid DNA using a standard Calcium Phosphate transfection technique and incubated overnight for 16-20 hours. Cells were then allowed to recover for one day. Resazurin reduction assay (FIG. 3) was used to estimate the number of viable cells in the 96-well plates. Resazurin is a cell permeable redox indicator that can be used to monitor viable cell number. Resazurin was dissolved in physiological buffers (resulting in a deep blue colored solution) and added directly to cells in culture in the 96-well plates in a homogeneous format. Viable cells with active metabolism can reduce resazurin into the resorufin product which is pink and fluorescent. Further, the quantity of resorufin produced is proportional to the number of viable cells which can be quantified using a microplate fluorometer equipped with a 535 nm excitation/590 nm emission filter set.

Referring to FIG. 4, two days after the plasmid DNA transfection, most cells transfected with the plasmids encoding fusion hExo1-Cas9 proteins had statistically increased cellular viability (about 3-4 folds) compared to cells transfected with control plasmids encoding unmodified Cas9 enzymes. Further, cells treated with HDR templates, a control antibody, or GFP plasmids and cells received no treatment have similar cellular viability.

### Example 2—Reduced Cellular Toxicity in A549 Cells with gRNA Targeting HBB Gene

Similarly to experiments conducted in Example 1, several plasmids containing polynucleotide encoding fusion protein hExo1-Cas9 enzymes (FIG. 1) were generated. Each of the plasmid constructs also contained a nucleotide sequence encoding a gRNA directed to recognize exon 1 of the human HBB gene. Compared to the experiments conducted in Example 1, an additional control of transfecting cells with plasmids with a nucleotide sequence encoding wildtype Cas9 enzyme and a nucleotide sequence encoding hExo1separately was incorporated. Three gRNA sequences as listed in Table 2 directed to recognize one of HBB gene's exon were used. Control plasmids were prepared to encode unmodified Cas9 (any of SEQ ID NO: 2-18) enzyme.

Similar cell culture and transfection protocols were used as in experiments conducted in Example 1. Referring to FIG. 6, gRNA G3 (SEQ ID NO: 23) has the highest cellular toxicity compared to gRNA G1 (SEQ ID NO: 21) and gRNA G2 (SEQ ID NO: 22). Referring to FIG. 8, cells transfected with RNP plasmids in general had higher percentage of viable cells compared to cells with the two control treatments. Further, FIG. 9B shows that RNP plasmids with the seventh fusion protein (FIGS. 1) and G2 gRNA had less cellular toxicity compared to RNP plasmids with the unmodified Cas9 and G3 gRNA.

### Example 3—Treating Sickle Cell Anemia in a Patient

A biological sample is obtained from a subject afflicted with sickle cell anemia. Genomic DNA is extracted from the biological sample and sequenced to verify a single nucleotide substitution (A to T) in the amino acid 6 codon of the β-globin gene. This mutation converts a glutamic acid codon (GAG) to a valine codon (GTG). Hematopoietic stem cells are isolated from the bone marrow cavity of the patient and cultured ex vivo. Nucleic acid vectors encoding the protein fusion complex of the hExo1-Cas9 and the gRNA moiety are delivered into the cultured hematopoietic stem cells. Further, the DNA template sequences with an integration cassette encoding the wild type sequence of exon 1 of β-globin gene are delivered to the cultured hematopoietic stem cells. The gRNA moiety comprises at least 10 nucleotides complementary to the GTG locus of exon 1 of the β-globin gene. The DNA template sequence comprises an integration cassette flanked by a 5′ homology region and a 3′ homology region, wherein the 5′ homology region and the 3′ homology region exhibit at least 80% identity to the segments flanking the GTG locus of exon 1. The integration cassette of the polynucleotide comprises a wild type GAG sequence that corresponds to the locus of chromosomal abnormality as detected in the primary cells. Upon delivering of the nucleic acids encoding the RNPs and DNA template sequences into the cultured hematopoietic stem cells, the gRNA directs the engineered hExo1-Cas9 proteins to the GTG locus, where the Cas9 portion of the engineered hExo1-Cas9 proteins creates a DSB. The hExo1portion of the engineered hExo1-Cas9 proteins partially digests the cleaved GTG locus of, leaving a 3′ overhang. The presence of the DNA template sequences promotes endogenous repair through HDR, where the integration cassette with the correct wild type sequence, GAG, at amino acid 6 of exon 1 of the β-globin gene is inserted into the chromosome of the hematopoietic stem cells. Hematopoietic stem cells with corrected GAG sequence is screened for and selected to be transplanted back into the patient.

### Example 4—Reduced Cellular Toxicity in A549 Cells with gRNA Targeting Intergenic Region on Chromosome 12

Similarly to experiments conducted in Example 2, several plasmids containing polynucleotide encoding fusion protein hExo1-Cas9 enzymes (FIG. 11A and FIG. 1) were generated. Each of the plasmid constructs also contained a nucleotide sequence encoding a gRNA directed to recognize an intergenic region on Chromosome 12, of which A549 cells have two copies. Compared to the experiments conducted in Example 3, the control of transfecting cells with plasmids with a nucleotide sequence encoding wildtype Cas9 enzyme and a nucleotide sequence encoding hExo1separately were also incorporated. Control plasmids were prepared to encode unmodified Cas9 (any of SEQ ID NO: 2-18) enzyme.

Similar cell culture and transfection protocols were used as in experiments conducted in Example 2. Roughly 2.5*10\hat{ }4 cells were plated in 96 well plates, with 8-16 replicates per individual experiment, as diagramed in FIG. 11B. Referring to FIG. 11C, cells transfected with PX330 plasmids in general had a much higher percentage of viable cells compared to cells with the two control treatments, 3-4 fold increase in cellular viability. FIG. 11C also shows that it is the fusion of hExo1that causes the decrease in cellular toxicity as the co-expression of Cas9 and hExo1do not affect cellular toxicity. FIG. 11D shows that treatment with alpha pfithrin of cells transfected with wild type Cas9 reduces the toxicity caused by the activity of Cas9. The inactivation of the Cas9 shown in FIG. 11D indicates that the cause of toxicity of Cas9 treatment in A549 cells is at least partly due to activation of P53 based on apoptosis, the same as in Ihrey et al. and Haapaniemi et al.

### Example 5—Quantification of HDR and INDELs rates of hExo-Cas9 Fusions in A549 Cells

Cas-9 hExo1fusions were used to integrate an antibiotic resistance cassette into a locus on Chromosome 6 of A549 cells. The Puromycin resistance repair template is diagramed in FIG. 12A. It contains a 5′ Homology Arm (5′), a strong constitutive viral promoter (pCMV), a Puromycin Resistance gene (Puro), a poly-A sequence (SV40Pa), and a 3′ Homology Arm (3′). Below the repair template shows the genomic region targeted by guides Int-G2 and G-3. The repair template is designed to disrupt integrate in the middle of both guide sequences, thereby preventing further Cas9 cleavage. The success of the integration is quantified by antibiotic selection. A549 cells have only one copy of Chromosome 6. The target integration site is ˜1 kb to the 3′ end of the human H2B gene on Chromosome 6. The region has no predicted genes.

Similar cell culture and transfection protocols were used as in experiments conducted in Example 4. Roughly 2.5*10\hat{ }4 cells were plated in 96 well plates, with 8-16 replicates per individual experiment, as diagramed in FIG. 12B.

FIG. 12C shows that Cas9-HR8 with G2 gRNA and G3 RNA, respectively, showed the greatest survival rate of A549 cells in Day 2 as compared to the other fusion proteins and Cas9. Due to this result, Cas9-HR8 was used in the following example.

### Example 6—Quantification of HDR and INDELs Rates of hExo-Cas9 Fusions in K562 Cells

Compared to the experiment in Example 5, K562 cells were used and Neon (Thermo Fisher) electroporation was used. K562 cells lack P53 function. In light of the results of Example 5, it was important to remove the variable of the activation of P53 by the activity of Cas9 as this would differ between fusion Cas9 and wild type Cas9, introducing the possibility of effecting the results of the antibiotic screen.

K562 cells were electroporated with 500 ng of each plasmid and 100 ng of repair template as shown in FIG. 12D. After two days, DNA is extracted from ˜ 1/10 of surviving cells and used for analysis of Puro RT genomic integration. The following day, 0.5 mg/mL Puromycin is added and after three days cellular survival is quantified with the standard resazurin assay as shown in FIG. 12D.

Quantification of toxicity was performed as in Example 5, with the addition of fusion construct 9. FIG. 12F shows a dramatic reduction in cellular toxicity between Cas9-HR 8 (with gRNA G2 and G3) as compared to Cas9 (with gRNA G2 and G3), double the amount of surviving cells.

Successful amplification using primer specific for the genome and specific for the repair template demonstrates successful integration of the repair template and that the reduction in toxicity of the Cas9-HR series of constructs is not due to lack of nuclease activity. There may be indication that the Cas9-HR series has a higher editing efficiency than Cas9.

FIG. 12E shows a depiction of the genomic region of cells which successfully integrated the repair template. Transfected cells are quantified on day 2. DNA is extracted from one well of each treatment. After 7 days of 1 microgram/milliliter puromycin treatment, cells are quantified with Resazurin. DNA is extracted from another row of cells. The insertion junctions are amplified with left and right primer pairs. Deep sequencing can be performed to identify INDEL rates in cells with successful HDR. DNA from Day 2 is used to quantify INDEL rates in HDR-cells by amplification with the left and right primer as seen in FIG. 12E.

FIG. 12G is depicts the results of gel electrophoresis on the amplification products, it shows that K562 cells transfected with either Cas9-HR8 (8) or Cas9 with gRNA G2 or G3 (NT) successfully produced amplicons with both primer pairs depicted in FIG. 12E while either GFP or transfected cells did not. This indicates that the repair template was successfully integrated.

### Example 7—Determining Relationship between Toxicity and Cas9 Activity

As seen in Example 2, different guide RNAs can have radically different cleavage rates and toxicities. Constructs with unmodified Cas9 and guides targeting regions shown in FIG. 13A were transfected into A549 cells using the same method as Example 4. Toxicity was quantified using Resazurin as in Example 1.

DNA was extracted from cells transfected with HBB-G1, HBB-G2, and HBB-G3, amplified with the outer primer pair in FIG. 13D and sent for Sanger sequencing. Only HBB-G3 showed significant cleavage as evidenced by the characteristic increase in noise following the cut-site shown in FIG. 13B. This indicates that toxicity is a good proxy for Cas9 nuclease activity in A549 cells. Guide RNA HBB-G3 was therefore used in Example 8.

### Example 8—Editing known Disease Loci with Cas9-HR

Similar to Example 7, K562 cells are used because they lack P53 activity as well as because they share more similarities to hematocytes than A549 cells.

The gRNA of Example 7, HBB-G3, is transfected with Cas9 and Cas9-HR 1-9 respectively to introduce multiple mutations into the HBB locus of K562 cells. The first mutation chosen is Sickle Cell E6V mutation. The Sickle Cell E6V mutation is made along with an additional mutation creating an EcoRI restriction site and two silent mutations designed to prevent re-cutting of the repair template once integrated into the genome, in addition to 60bp homology arms on each side of the predicted cut-site.

Transfection is achieved with electroporation. Two days after electroporation, toxicity assays with Resazurin are conducted as in Example 6. DNA is also harvested and the HBB locus is amplified to prepare for deep sequencing to measure INDELs and HDR rate. Alternatively, DNA can be digested with EcoRI to measure target efficiency. FIG. 16A and FIG. 16B illustrate that upon integration of the repair template, the genomic locus can now be digested with EcoRI. EcoRI digested amplicons can be observed in Cas9-HR4, Cas9-HRS, Cas9-HR6, Cas9-HR7, and Cas9-HR8 lanes. FIG. 16C, FIG. 16D, and FIG. 16E confirm that Cas9-HR is expressed and localized to the nucleus of the transfected cells.

### Example 9-Editing CD34+ Hematopoietic Stem Cells

The experiments of Example 8 are repeated on CD34+ cells. The gRNA from Example 8, HBB-G3, is transfected with Cas9 and Cas9-HR 1-9 respectively to introduce multiple mutations into the HBB locus of K562 cells. The first mutation chosen is Sickle Cell E6V mutation. The Sickle Cell E6V mutation is made along with an additional mutation creating an EcoRI restriction site and two silent mutations designed to prevent re-cutting of the repair template once integrated into the genome, in addition to 60 bp homology arms on each side of the predicted cut-site.

Transfection is achieved with electroporation. Two days after electroporation, toxicity assays with Resazurin are conducted as in Example 6. DNA is harvested and the HBB locus is amplified to prepare for deep sequencing to measure INDELs and HDR rate. Alternatively, DNA is digested with EcoRI to measure target efficiency.

### Example 10-In-Vitro Nuclease Activity of Cas9-HR3

A 954 bp piece of DNA was amplified from wildtype K562 cells using standard Taq DNA polymerase and HBB-out-4-F (5′-aacgatcctgagacttccaca-3′ (SEQ ID NO: 127)) and HBB-out-5-R (5′-tgcttaccaagctgtgattcc-3′ (SEQ ID NO: 128)), Tm=56 for 35 cycles, and purified using the Qiagen PCR cleanup kit. Next, HBB-G1 (5′-guaacggcagacuucuccuc-3′ (SEQ ID NO: 129), IDT) or HBB-G3 (5′-gaggugaacguggaugaagu-3′ (SEQ ID NO: 130), IDT) were combined with tracrRNA (IDT) at final concentrations of 1μM each in duplex buffer (IDT). The RNA was heated for 5 minutes at 95° C., then allowed to cool to room temperature. Cas9 or Cas9-HR3 were then combined with either HBB-G1 or HBB-G3 guide RNA complex and amplified DNA at a 10:10:1 molar ratio (30 nM: 30nM: 3nM) in 1X Cas9 reaction Buffer (50 mM Tris, 100 mM NaCl, 10 mM MgCl2, 1 mM DTT, pH7.9) and incubated for 1 hr at 37° C., after which 1 μL of Proteinase K was added and the reaction was incubated for an additional 20 minutes at 50° C. The samples were then electrophoresed on a standard 1% TAE agarose gel and imaged.

FIG. 18A illustrates the mechanistic modeling of the Cas9-HR. Cas9 binds to the intended site, cuts, and then remains bound until digested away with proteinase K. As Cas9-HR possesses additional 5′->3′ exonuclease activity, a more complex pattern is expected. Importantly, it has been shown that hExo1 has roughly 10× the affinity for phosphorylated 5′-double strand DNA ends as for unphosphorylated. This leads to two important consequences. First, it is expected that there would be some small digestion of the PCR without addition of any gRNA, which is not generally expected to happen with Cas9. Changing the nature of the primers used to amply the DNA fragment (either with 5′-phosphates or thioester bonds) can either increase or decrease this degradation respectively. Second, since cleavage of double-stranded-DNA (dsDNA produces ends with 5′-phosphates, it is expected that either the original Cas9-HR or other unbound Cas9-HR molecules resect the dsDNA in 5′->3′ generating a mix of various dsDNA, double stranded and single-stranded (ds::ss) DNA, and ssDNA products. FIG. 18B illustrates am anticipated Cas9 and Cas9-HR digestion pattern based on the mechanism of FIG. 18A. FIG. 18C illustrates an actual agarose example of FIG. 18A and FIG. 18B. Lanes 1 and 2 show Cas9-HR3 targeting either HBB-G1 or HBB-G3, Lanes 3 and 4 show Cas9 (NT) targeting either HBB-G1 or HBB-G3, Lane 5 is Untreated DNA. FIG. 18D illustrates a similar experiment as FIG. 18C and differs from FIG. 18B by conducting the experiment after leaving enzymes for 2 weeks at 4° C. in order to compare protein stability. Lane 1 is digestion pattern from the combination of Cas9-HR3 and gRNA HBB-G1. Lane 2 is digestion pattern from the combination of Cas9 and gRNA HBB-G1. Lane 3 is digestion pattern from the combination of Cas9-HR3 and HBB-G3. Lane 4 is digestion pattern from the combination of Cas9 and HBB-G3. Lane 5 is digestion pattern from Cas9-HR only. Lane 6 is digestion pattern from Cas9 only. Lane 7 is the control where there is neither Cas9 nor gRNA. FIG. 18C and FIG. 18D demonstrate that the digestion pattern correspond to the mechanism as shown in FIG. 18A and FIG. 18B.

### Example 11- hH2B Genomic Integration and Genomic Validation

Cas9-HR and Cas were utilized to introduce an hH2b fragment into the H2B genomic locus. Primers were designed so that the genomic primer is outside of the H2B-mNeon repair template (RT), while the other is RT specific (within mNeon) as shown in FIG. 19A. Sequences for 3′ primers are H2B-RT-3′-F: 5′-aggcctttaccgatgtgatg-3′ (SEQ ID NO: 131), H2B-RT-3′-R:5′-acggagtctcgctctgtcac-3′ (SEQ ID NO: 132). Sequences for 5′ primers are H2B-RT-5′-F: 5′-caaactgcaaggctgcaata-3′ (SEQ ID NO: 133), H2B-RT-3′-R: 5′-gacccaccatgtcaaagtcc-3′ (SEQ ID NO: 134)

After transfection of K562 cells, genomic DNA was extracted from cells transfected with repair template (RT) and either Cas9-HR4, Cas9-HR8, Cas9 (NT), or untransfected (Con). Standard Taq polymerase (Bioneer, Tm=56, 35 cycles) was used to amplify the fragments flanked by the 5′ primers or the 3′ primers.

FIG. 19B illustrates an agarose gel showing PCR products amplified by the 5′ primers. Amplification products were detected for Cas9-HR4,8 and Cas9-NT, but were not detected in the un-transfected control.

FIG. 19C illustrates an agarose gel showing successful specific amplification by the 3′ primers in Cas9-HR4, Cas9-HR8, and Cas9 only (NT).

FIG. 19D illustrates absorbance of sequence trace from Sanger sequencing of the PCR product of Cas9-HR8 amplified by the 5′ primers. Solid or unfilled bars bellowed the called base denote identity of DNA (top left bar without fill is genomic, two bars in the middle with stripes are H2B ORF, and the bottom right bar without fill is mNeon), with the vertical grey dashed line of FIG. 19A showing the junction between genomic sequences included in the RT vs solely endogenous genomic sequences. Clear transition from H2B to mNeon sequences included in the RT to solely endogenous genomic sequence indicated successful integration of the transgene at the 5′ end.

FIG. 19E illustrates absorbance of sequence trace from Sanger sequencing of the PCR product of Cas9-HR8 amplified by the 3′ primers. Bars above the called bases denote identity of DNA (unfilled bar is mNeon and shaded bar is genomic). Clear transition from mNeon to genomic sequences included in the RT to solely endogenous genomic sequence indicated successful integration of the transgene at the 3′ end.

FIG. 19F and FIG. 19G illustrate alignment of sequencing results the 5′ (FIG. 19F) and 3′ (FIG. 19G) PCR products from Cas9-HR4, Cas9-HR8, and NT with the reference sequence. Sequences were aligned using ClustalOmega.

### Example 12—Editing Adipose or Pre-Adipose Tissue to Increase Metabolic Flux

Cells from either undifferentiated or mature adipose tissue are isolated from a patient and transfected with either plasmids encoding any one of the versions of Cas9-HR or purified RNPs. The chosen Cas9-HR(s) can be targeted to sites of the human genome which have been already been shown to be amenable to DNA insertion (“safe harbor sites”) or any such novel site identified. Additionally, a repair template containing the cDNA for either Uncoupling Proteins (UCPs) 1, 2, 3 is transfected simultaneously. This transgene contains 5′ Homology Arms (HAs) to the chosen integration site, either a ubiquitous or tissue specific enhancer complexed with a basal promoter, either with or without 5′UTR sequence, an ORF consisting of the aforementioned cDNA from either UCP 1, 2 or 3, with or without a 3′ UTR sequence, a poly-adenylation sequence, and a 3′ HA to the chosen integration site. Integration and subsequent reintroduction of the Adipose Tissue expressing this transgene can increase basal metabolism, leading to overall weight loss and decrease in adipose lipid deposit size. Use of Cas9-HRs can lead to reduction in toxicity and increase the number of cells successfully integrated.

### Example 13-Editing Human Dermal Cells to Decrease Androgenic Alopecia

Plasmids encoding Cas9-HR(s) or purified RNPs can be used to transfect either isolated cells or in-situ on the scalp to transfect transgenes expressing either full length or modified Sex Binding Hormone Globulin (SBHG), NRF 2, or SRD5A1, 2 or 3. The chosen Cas9-HR(s) can be targeted to sites of the human genome which have been already been shown to be amenable to DNA insertion (“safe harbor sites”) or any such novel site identified. These transgenes contain 5′ Homology Arms (HAs) to the chosen integration site, either a ubiquitous or tissue specific enhancer complexed with a basal promoter, either with or without 5′UTR sequence, an ORF consisting of the aforementioned cDNA from either SBHG, NFR2 or SRD5A1, 2, or 3, with or without a 3′ UTR sequence, a poly-adenylation sequence, and a 3′ HA to the chosen integration site. Successful transfection of either in-situ cells or re-introduction of isolated dermal cells can delay or permanently halt hair-loss and result in hair regrowth.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein may be employed. It is intended that the following claims define the scope of the present disclosure and that methods and structures within the scope of these claims and their equivalents be covered thereby.

### REFERENCES

1. Oakes, B. L., Nadler, D. C. & Savage, D. F. Protein engineering of Cas9 for enhanced function. Methods Enzymol. 546, 491-511 (2014).
2. Cong, L. et al. Multiplex Genome Engineering Using CRISPR/Cas Systems. Science 339, 819-823 (2013).
3. Ran, F. A. et al. Genome engineering using the CRISPR-Cas9 system. Nat. Protoc. 8, 2281-2308 (2013).
4. Jinek, M. et al. A Programmable Dual-RNA-Guided DNA Endonuclease in Adaptive Bacterial Immunity. Science 337, 816-821 (2012).
5. Eid, A., Alshareef, S. & Mahfouz, M. M. CRISPR base editors: genome editing without double-stranded breaks. Biochem. J. 475, 1955-1964 (2018).
6. Gehrke, J. M. et al. An APOBEC3A-Cas9 base editor with minimized bystander and off-target activities. Nat. Biotechnol. (2018). doi:10.1038/nbt.4199
7. Wang, L. et al. In Vivo Delivery Systems for Therapeutic Genome Editing. Int. J. Mol. Sci. 17, (2016).
8. Zhang, J.-P. et al. Efficient precise knockin with a double cut HDR donor after CRISPR/Cas9-mediated double-stranded DNA cleavage. Genome Biol. 18, 35 (2017).
9. Li, H. et al. Design and specificity of long ssDNA donors for CRISPR-based knock-in. bioRxiv 178905 (2017). doi:10.1101/178905
10. Canny, M. D. et al. Inhibition of 53BP1 favors homology-dependent DNA repair and increases CRISPR-Cas9 genome-editing efficiency. Nat. Biotechnol. 36, 95-102 (2018).
11. Liang, X., Potter, J., Kumar, S., Ravinder, N. & Chesnut, J. D. Enhanced CRISPR/Cas9-mediated precise genome editing by improved design and delivery of gRNA, Cas9 nuclease, and donor DNA. J. Biotechnol. 241, 136-146 (2017).
12. Ihry, R. J. et al. p53 inhibits CRISPR-Cas9 engineering in human pluripotent stem cells. Nat. Med. 24, 939-946 (2018).
13. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
14. Bieging, K. T., Mello, S. S. & Attardi, L. D. Unravelling mechanisms of p53-mediated tumour suppression. Nat. Rev. Cancer 14, 359-370 (2014).
15. Muller, P. A. J. & Vousden, K. H. Mutant p53 in cancer: new functions and therapeutic opportunities. Cancer Cell 25, 304-17 (2014).
16. Canny, M. D. et al. Inhibition of 53BP1 favors homology-dependent DNA repair and increases CRISPR-Cas9 genome-editing efficiency. 36, 95-102 (2018).
17. Ceccaldi, R., Rondinelli, B. & D'Andrea, A. D. Repair Pathway Choices and Consequences at the Double-Strand Break. Trends Cell Biol. 26, 52-64 (2016).
18. Lieber, M. R. The mechanism of double-strand DNA break repair by the nonhomologous DNA end-joining pathway. Annu. Rev. Biochem. 79, 181-211 (2010).
19. Shibata, A. et al. DNA double-strand break repair pathway choice is directed by distinct MRE11 nuclease activities. Mol. Cell 53, 7-18 (2014).
20. Tomimatsu, N. et al. Exo1 plays a major role in DNA end resection in humans and influences double-strand break repair and damage signaling decisions. DNA Repair 11, 441-8 (2012).
21. Bolderson, E. et al. Phosphorylation of Exo1 modulates homologous recombination repair of DNA double-strand breaks. Nucleic Acids Res. 38, 1821-1831 (2010).
22. Tomimatsu, N. et al. Phosphorylation of EXO1 by CDKs 1 and 2 regulates DNA end resection and repair pathway choice. Nat. Commun. 5, 3561 (2014).
23. Tomimatsu, N. et al. DNA-damage-induced degradation of EXO1 exonuclease limits DNA end resection to ensure accurate DNA repair. J. Biol. Chem. 292, 10779-10790 (2017).
24. Paudyal, S. C., Li, S., Yan, H., Hunter, T. & You, Z. Dna2 initiates resection at clean DNA double-strand breaks. Nucleic Acids Res. 45, 11766-11781 (2017).
25. Tomimatsu, N. et al. DNA-damage-induced degradation of EXO1 exonuclease limits DNA end resection to ensure accurate DNA repair. J. Biol. Chem. 292, 10779-10790 (2017).
26. Chapman, J. R., Taylor, M. R. G. & Boulton, S. J. Playing the End Game: DNA Double-Strand Break Repair Pathway Choice. Mol. Cell 47, 497-510 (2012).
27. Hu, Z. et al. Ligase IV inhibitor SCR7 enhances gene editing directed by CRISPR-Cas9 and ssODN in human cancer cells. Cell Biosci. 8, 12 (2018).
28. Ren, B. et al. Improved Base Editor for Efficiently Inducing Genetic Variations in Rice with CRISPR/Cas9-Guided Hyperactive hAID Mutant. Mol. Plant 11, 623-626 (2018).
29. Li, X. et al. Base editing with a Cpfl-cytidine deaminase fusion. Nat. Biotechnol. 36, 324-327 (2018).
30. Jiang, W. et al. BE-PLUS: a new base editing tool with broadened editing window and enhanced fidelity. Cell Res. 28, 855-861 (2018).
31. La Russa, M. F. & Qi, L. S. The New State of the Art: Cas9 for Gene Activation and Repression. Mol. Cell. Biol. 35, 3800-9 (2015).
32. Chang, H. H. Y., Pannunzio, N. R., Adachi, N. & Lieber, M. R. Non-homologous DNA end joining and alternative pathways to double-strand break repair. Nat. Rev. Mol. Cell Biol. 18, 495-506 (2017).
33. Jia, P.-P. et al. Role of human DNA2 (hDNA2) as a potential target for cancer and other diseases: A systematic review. DNA Repair 59, 9-19 (2017).
34. Orans, J. et al. Structures of human exonuclease 1 DNA complexes suggest a unified mechanism for nuclease family. Cell 145, 212-23 (2011).
35. Xiaoying, C., Jennica, Z. & Wei-Chiang, S. Fusion Protein Linkers: Property, Design and Functionality. Adv Drug Deliv Rev 65, 1357-1369 (2014).
36. Prabst, K., Engelhardt, H., Ringgeler, S. & Hubner, H. Basic Colorimetric Proliferation Assays: MTT, WST, and Resazurin. in Cell Viability Assays: Methods and Protocols (eds. Gilbert, D. F. & Friedrich, O.) 1-17 (Springer New York, 2017). doi:10.1007/978-1-4939-6960-9_1
37. Lieber, M., Todaro, G., Smith, B., Szakal, A. & Nelson-Rees, W. A continuous tumor-cell line from a human lung carcinoma with properties of type II alveolar epithelial cells. Int. J. Cancer 17, 62-70 (1976).
38. Klein, E. et al. Properties of the K562 cell line, derived from a patient with chronic myeloid leukemia. Int. J. Cancer 18, 421-431 (1976).

What is claimed is:

1. A composition comprising:
i) a polypeptide, comprising a first functional fragment, a second functional fragment comprising a Cas9 nuclease, and a linker peptide, wherein:
said first functional fragment is coupled to a first end of the linker peptide and the second functional fragment is coupled to a second end of said linker peptide; and
said first functional fragment comprises an exonuclease wherein said exonuclease is selected from the group consisting of MRE11, EXO1, EXOIII, EXOVII, EXOT, DNA2, CtIP, TREX1, TREX2, Apollo, RecE, RecJ, T5, Lexo, RecBCD, and Mungbean;
ii) a guide RNA (gRNA); and
iii) a homology directed repair (HDR) template.

2. The composition of claim 1, wherein said exonuclease is a human Exo1 enzyme.

3. The composition of claim 2, wherein an N-terminal of said human Exo1 enzyme is coupled to a C-terminal of said linker which is coupled to said C-terminal of said Cas nuclease.

4. The composition of claim 2, wherein said human Exo1 enzyme comprises at least 80% sequence identity to SEQ ID NO: 1.

5. The composition of claim 1, wherein said Cas9 nuclease comprises an N-terminal nuclear localizing sequence (NLS).

6. The composition of claim 1, wherein said Cas9 nuclease comprises a C-terminal nuclear localizing sequence (NLS).

7. The composition of claim 1, wherein said linker peptide is selected from a group consisting of FL2X, SLA2X, AP5X, FL1X, SLA1X.

8. The composition of claim 7, wherein said linker peptide is SLA2X.

9. The composition of claim 1, wherein said linker peptide comprises 5 to 200 amino acids.

10. A polynucleotide encoding the composition of any one of claims 1 to 9.

11. A vector comprising the polynucleotide of claim 10.
